(19) United States
(12) Patent Application Publication
(10) Pub. No.: US 2026/0084375 A1
(43) Pub. Date: Mar. 26, 2026

(54) SYSTEMS AND METHODS FOR FABRICATING BIOPRINTED FIBER STRUCTURES

(71) Applicant: ASPECT BIOSYSTEMS LTD., Vancouver (CA)

(72) Inventors: Sam Wadsworth, Vancouver (CA); Simon Beyer, Richmond (CA); Spiro Getsios, Nanaimo (CA); Sheng Pan, Richmond (CA); Navid Hakimi, Vancouver (CA); Keddie Brown, Vancouver (CA)

(21) Appl. No.: 18/866,497

(22) PCT Filed: May 15, 2023
(86) PCT No.: PCT/CA23/50667

§ 371 Date: Nov. 15, 2024

Related U.S. Application Data
(60) Provisional application No. 63/342,118, filed on May 15, 2022.

(51) Int. Cl. B29C 64/245; A61L 27/20; A61L 27/34; A61L 27/54; B29C 64/118; B29C 64/209; B29C 64/393; B29C 71/00; B29K 105/00; B29L 31/00; B33Y 10/00; B33Y 30/00; B33Y 40/20; B33Y 50/02; B33Y 80/00

(52) U.S. Cl. CPC B29C 64/245; A61L 27/20; A61L 27/34; A61L 27/54; B29C 64/118; B29C 64/209; B29C 64/393; B29C 71/0009; B33Y 10/00; B33Y 30/00; B33Y 40/20; B33Y 50/02; B33Y 80/00; A61L 2300/64; B29K 2105/0035; B29L 2031/753

(57) ABSTRACT

Aspects of the disclosure include a fabrication platform for supporting a bioprinted fiber structures during printing, patterning, and/or processing, comprising a frame with a plurality of posts for securing a cross-linkable fiber during printing thereof, and where a continuous length of the cross-linkable fiber is printed around a plurality of posts during the 3D bioprinting process. The fabrication platform enables the cross-linkable fiber to be suspended during one or more of printing, patterning, and/or processing. In this way, the bioprinted fiber structure comprises a uniform outer surface, and can be easily modified and/or further processed after printing and patterning are completed.

### CROSS-REFERENCE TO RELATED APPLICATIONS

This application claims priority to U.S. Provisional Patent Application No. 63/342,118, filed May 15, 2022, the contents of which are incorporated herein by reference in their entirety.

### FIELD OF DISCLOSURE

The present disclosure relates generally to three-dimensional (3D) printing and the generation of three-dimensional biological structures from digital files. Specifically, the invention relates to systems and methods for generating a cross-linkable fiber structure having a uniform external surface during printing and patterning, and to facilitate post-printing modifications thereto.

### BACKGROUND OF THE DISCLOSURE

The tissue engineering art has long sought to fabricate viable synthetic structures and devices capable of mimicking the function of a target tissue or organ using myriad materials and methods. Unfortunately, however, the practical realization of these synthetic structures still faces a number of significant challenges. Suitable synthetic devices must protect the encapsulated cells and/or tissue fragments from the host's immune system without hindering the passage of nutrients, oxygen, and secreted products (e.g. insulin). Moreover, the materials employed in creating the synthetic structure must be biocompatible and have sufficient strength and elasticity to survive for prolonged periods of time in vivo, but without triggering an adverse immunologic reaction.

In particular, implantation into the body can trigger an orchestrated biological response by both innate and adaptive immune systems against a device with the intention of neutralizing it, referred to as a foreign body response (FBR). The cellular response against perceived pathogens that are too large to be phagocytosed is mediated in part by macrophages that overexpress extracellular matrix (ECM) proteins, such as fibronectin, and also produce pro-fibrogenic factors that enhance fibrogenesis by fibroblasts, resulting in the formation of a fibrotic capsule around the device. This fibrous capsule can interfere with device function, particularly when they contain therapeutic cell populations which require access to nutrients and oxygen flow in order to perform their intended function.

A wide range of materials from naturally occurring polymers to synthetic materials have been described as generating fibrotic responses (Ward W K, J Diabetes Sci Technol. (2008); 2: 768-777; Zhang L et al., Nature Biotech. (2013); 31: 553-556; Ratner B D, Journal of Controlled Release. (2002); 78: 211-218). Furthermore, physical parameters such as the shape, size, stiffness, and texture of the synthetic tissue structures are intrinsic properties that are also known to contribute to FBR. For example, the surface of a synthetic tissue structure can affect the behavior of macrophages and other immune cells, with structures lacking sharp edges and a smooth surface generally being more biocompatible and inducing less inflammation (Mariani E et al., Int J Mol Sci. (2019); 20: doi: 10.3390/ijms20030636; Salthouse T N, Journal of Biomedical Materials Research Part A. (1984); 18: 395-401. Moreover, changes in surface roughness at the nanoscale have also been associated with increased protein adsorption (Hulander M et al., Int J Nanomedicine. (2011); 6: 2653-2666; Roach P., J Mater Sci Mater Med. (2007); 18: 1263-1277; Scopelliti P E et al., PLOS ONE. (2010); 5: e11862; Rechendorff K et al., Langmuir. (2006); 22: 10885-10888; Hovgaard M B et al., J Phys Chem. B (2008); 112: 8241-8249), and different nanostructured topographies can affect cellular interactions (Baker D W et al., Biomacromolecules. (2011); 12: 997-1005; Jahed Z., Biomaterials. (2014); 35: 9363-9371).

Accordingly, improvements in both design and materials are still needed to accommodate the opposing objectives of immune protection and nutrient passage, and to help mitigate the FBR response. As such, there is a need for synthetic tissue structures that effectively balance capabilities that reduce or avoid immune system recognition and/or breach of such synthetic tissue structures with capabilities that ensure adequate passage of oxygen and nutrients to cells of the synthetic structure. There is also a need for synthetic structures and methods of generation thereof, in which patterning is consistent and reliable, where the structures have sufficient strength and elasticity to survive in vivo for prolonged periods of time, and are readily retrievable.

### SUMMARY OF DISCLOSURE

The present invention addresses the foregoing shortcomings in the prior art with a fabrication platform, a means for suspending a bioprinted fiber structure, bioprinting systems incorporating same, and methods of use thereof for the generation of tissue fiber structures with anti-FBR properties and improved structural stability. As disclosed and demonstrated herein for the first time, 3D bioprinted fiber structures are generated in a manner that reduces or avoids contact with a receiving surface during printing, patterning and/or processing. This in turn can significantly reduce or eliminate the introduction of surface imprints or other imperfections, facilitate transport and/or manipulation thereof, and allow for conformal coating of the entire external surface of a bioprinted fiber structure with materials that can impart desired properties such as enhanced stability and/or anti-FBR.

Aspects of the invention include a fabrication platform for supporting a bioprinted fiber structure during printing, patterning and/or processing, wherein said platform comprises a frame defining a void and comprising a plurality of posts on opposing sides of the frame for securing and suspending at least one cross-linkable fiber within said frame to form said fiber structure, wherein a continuous length of said at least one fiber is printed around at least two, three, four, five, six, seven, eight, nine, ten or more of said posts during the 3D bioprinting process.

In embodiments, said posts are positioned on the interior of said frame; preferably wherein said posts are positioned on frame projections extending into said void. In embodiments, the reliance on the projections minimizes contact between the fiber and the frame.

In embodiments, said posts are evenly or unevenly spaced around said frame.

In embodiments, said frame comprises at least 10, 15, 20, 25, 30, 35, 40, 45, 50, 55, 60, 65, 70, 75, 80, 85, 90, 95, or 100 posts, preferably between about 0.5 mm to about 50 mm in height.

In embodiments, at least a portion of said cross-linkable fiber comprises a biological material.

In embodiments, said frame is triangular, rectangular, hexagonal, octagonal, circular, etc.

In embodiments, said fiber structure comprises a lattice.

In embodiments, said frame is coupled to a mounting bracket configured for adjusting a position of said frame with respect to a receiving surface. In embodiments, the frame further comprises a fiber cutting groove positioned alongside the void to permit a cutting implement to cut a portion of the fiber structure. In embodiments, the frame further comprises a mating groove disposed on a bottom surface of the frame and configured to receive the walls of a corresponding container or vessel on the receiving surface.

Aspects of the invention include a means for suspending a bioprinted fiber structure during printing, patterning and/or processing, wherein said means for suspending comprises a frame coupled to a mounting bracket and/or a receiving surface of a bioprinting system, said frame comprising a plurality of posts encircling the frame for securing a continuous length of at least one cross-linkable fiber forming said fiber structure.

Aspects of the invention include a bioprinting system. In embodiments, the bioprinting system comprises a fabrication platform as herein disclosed, or a means for suspending a bioprinted fiber structure as herein disclosed. In embodiments, the bioprinting system comprises at least one dispensing orifice for dispensing said at least one crosslinkable fiber onto said receiving surface. In embodiments, the bioprinting system comprises a positioning unit for positioning the receiving surface in three dimensional space with respect to the dispensing orifice, the positioning unit operably coupled to either the receiving surface or the at least one dispensing orifice. In embodiments, the bioprinting system comprises a dispensing means for dispensing the at least one cross-linkable fiber from the at least one dispensing orifice.

In embodiments of the bioprinting system, said fabrication platform or said means for suspending is suspended over said receiving surface.

In embodiments, said receiving surface comprises a porous material.

In embodiments, said receiving surface comprises a vessel containing a liquid, e.g., a cross-linker bath or optional dipcoat, preferably positioned on or around a vacuum chuck. In embodiments, the receiving surface comprises a vacuum chuck and integrated container formed by walls projecting from the top surface of the vacuum chuck and defining the perimeter of the integrated container. In embodiments, the walls of the integrated container are configured to insert into a mating groove on the bottom of the frame, preferably in form-fit fashion to prevent leakage of fluid over the walls when the frame is placed on the container.

In embodiments, the bioprinting system further comprises a programmable control processor for controlling the positioning component and for controlling a flow rate of one or more fluids via the dispensing means.

In embodiments, said dispensing means comprises at least one pump; optionally wherein said at least one pump comprises a pump assembly comprising a plurality of pumps positioned in a radial array on a mounting bracket.

In embodiments, the bioprinting system further comprises at least one print head comprising a plurality of microfluidic printing channels to selectively provide a respective plurality of materials.

Aspects of the invention include a method for bioprinting a fiber structure. In embodiments, the method comprises providing a bioprinting system as herein disclosed, and dispensing a continuous length of the cross-linkable fiber around a plurality of said posts on said frame of said fabrication platform to generate the fiber structure.

In embodiments, the method further comprises adding a conformal coating to an entirety of an external surface of the fiber structure while said fiber remains coupled to said frame.

In embodiments, the method further comprises transporting the fiber structure from one location to another while said fiber structure remains coupled to said frame.

In embodiments, the method further comprises storing said fiber structure while said fiber structure remains coupled to said frame.

Aspects of the invention include a bioprinted fiber structure made by a method of the present disclosure. In embodiments, the bioprinted fiber structure comprises a continuous length of the cross-linkable fiber comprising at least one biological material, wherein said cross-linkable fiber comprises a solid core and at least one external shell layer surrounding said solid core, wherein said bioprinted fiber structure comprises at least two layers of a lattice/grid formed by said continuous cross-linkable fiber.

In embodiments, each layer is about 0.050 mm to about 3 mm thick.

In embodiments, the bioprinted fiber structure has an infill density of between about 10% and about 90%, or between about 20% and about 80%, or between about 30% and about 70%, or between about 40% and about 60%, preferably about 30%, about 40%, about 50%, or about 60%.

In embodiments, the solid core comprises at least one biological material, optionally wherein said solid core is compartmentalized along the length of said fiber.

In embodiments, the bioprinted fiber structure comprises at least one internal shell layer surrounding said solid core, said at least one internal shell layer comprising at least one biological material; optionally wherein said solid core and/or said at least one internal shell layer is/are compartmentalized along the length of said fiber.

In embodiments, the bioprinted fiber structure comprises at least one conformal coating.

In embodiments, said solid core comprises about about 1.5% alginate, said at least one external shell layer comprises about 2.0% alginate, and said coating comprises about 0.5% alginate.

In embodiments, the bioprinted fiber structure comprises an inner conformal coating and an outer conformal coating.

In embodiments, the biological material comprises pancreatic islet cells.

Other features, objects, and advantages will be apparent from the disclosure that follows.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 depicts an exemplary illustration of a print head of a microfluidics-based bioprinting system dispensing a fiber structure onto a receiving surface.

FIGS. 2A-2E depict examples of means for suspending a bioprinted fiber structure in accordance with embodiments of the present disclosure.

FIG. 3 is an illustration exemplifying how the means for suspending a bioprinted fiber structure as depicted in FIG. 2 can be used to bioprint a fiber structure.

FIG. 4 is an image of an illustrative fiber structure comprised of two layers printed via the means for suspending a bioprinted fiber structure of FIG. 2.

FIGS. 5A-5D depict illustrative embodiments of fabrication platforms of the present disclosure.

FIGS. 5E-5F illustrate a fabrication platform including an integrated container, and a corresponding frame including a groove for receiving the walls of the integrated container, respectively.

FIG. 5G illustrates the sequential liquid coating of the bottom and top surface of a fiber structure on a frame positioned on the integrated container of the present disclosure.

FIG. 5H illustrates a standalone vacuum chuck and integrated container of the present disclosure.

FIGS. 5I-5K depict additional illustrative embodiments of fabrication platforms of the present disclosure.

FIG. 6 is an illustration exemplifying how a means for suspending a bioprinted fiber structure such as that of FIGS. 2A-2C can be used to print a fiber into a bath solution.

FIG. 7 is an illustration of a fabrication platform that includes a vessel for bioprinting a fiber structure into a bath.

FIG. 8 is an illustrative process flow for coating a fiber structure created via a means for suspending a bioprinted fiber structure according to embodiments.

FIG. 9A is an image of a bioprinted fiber structure coupled to a means for suspending a bioprinted fiber structure according to embodiments.

FIG. 9B is an image of the bioprinted fiber of FIG. 7 following removal from the means for suspending a bioprinted fiber structure.

FIGS. 10A-D are images of bioprinted fiber structures in accordance with embodiments of the present disclosure.

FIG. 11A is a schematic of a 10×10 mm fiber structure of the present disclosure.

FIG. 11B is an image of a coated 10×10 mm fiber structure coupled to frame of the present disclosure.

FIGS. 11C-11D depict images of the coated 10×10 mm fiber structures of FIGS. 11A-11B uncoupled from the frame.

FIG. 12A depicts live/dead staining of a coated 10×10 mm fiber structure as compared to a coated 18×18 mm fiber structure each comprising HepG2 aggregates, assessed at 0 days following printing.

FIG. 12B depicts live/dead the coated 10×10 mm fiber structure as compared to a coated 18×18 mm fiber structure each comprising HepG2 aggregates, assessed at 5 days following printing.

FIGS. 13A-13B show images of a 10×10 mm lattice structure after coating, on frame (FIG. 13A), and uncoupled from frame (FIG. 13B).

FIG. 14 summarizes stability data for coated 10×10 mm fiber structures comprising HepG2 aggregates or primary rat islets (PRI).

FIGS. 15A-15C depict images of three coated 10×10 mm fiber structures with HA-containing cores.

FIGS. 15D-15F depict images of three coated 10×10 mm fiber structures without HA-containing cores (i.e., SLG100 but not HA).

FIG. 16A illustrates live/dead staining of coated 10×10 mm fiber structures loaded with PRIs, assessed at 0 days post printing.

FIG. 16B illustrates live/dead staining of the coated 10×10 mm fiber structures of FIG. 16A, assessed at 3 days post printing.

FIG. 17 is a table illustrating stability data of fiber structures printed on a frame of the present disclosure and then coated as compared to in lieu of such a frame.

FIGS. 18A-18C are images of fiber structures printed on a frame of the present disclosure and then coated, following their being subjected to a stability test, the data of which is summarized at FIG. 17.

FIGS. 18D-18F are images of fiber structures printed in lieu of a frame of the present disclosure and then coated, following their being subjected to a stability test, the data of which is summarized at FIG. 17.

FIG. 19A is a microscopic image of a coated fiber structure printed by way of a frame of the present disclosure.

FIGS. 19B-19C are microscopic images of coated fiber structures printed in lieu of a frame of the present disclosure.

FIG. 20 is an image depicting inside-out cross linked, uncoated bioprinted fiber structures, printed in lieu of a frame of the present disclosure.

FIG. 21A is a schematic representation and bright field image of bioprinted primary human islet tissue.

FIG. 21B illustrates live/dead staining of bioprinted primary islets.

FIG. 21C depicts data from a Glucose-Stimulated Insulin Secretion (GSIS) assay performed using primary human and rat islets.

FIG. 22A illustrates random-fed blood glucose measurements following streptozotocin (STZ) treatment and intraperitoneal (IP) implantation of bioprinted human islet tissue in NSG (NOD scid gamma) mice over 80 days.

FIG. 22B illustrates human C-peptide levels measured in mouse plasma over 80 days using ELISA.

FIG. 22C illustrates data from an oral glucose tolerance test (OGTT) performed at day 80 to assess kinetics of blood glucose normalization following a fasting period and subsequent glucose challenge in NSG mice with bioprinted islet tissues or healthy, non-STZ treated control mice.

FIG. 23A illustrates blood glucose measurements following omental pouch implantation of bioprinted rat islet tissue in STZ-treated nude rats (n=2) over 180 days.

FIG. 23B shows H&E (high and low magnification) and insulin (islets) or CD31 (endothelial cells) immunohistochemistry (IHC) performed on sections from fixed, bioprinted tissue explanted at 180 days.

FIG. 24A illustrates blood glucose measurements following omental pouch implantation of bioprinted Lewis rat islet tissue in STZ-treated Sprague-Dawley (SD) rats over 90 days.

FIG. 24B shows H&E and insulin (islets) or CD31 (endothelial cells) IHC performed on sections from fixed, bioprinted tissue explanted at 60 days.

FIG. 25A is a schematic illustration of a biomanufacturing process of the present disclosure.

FIG. 25B shows bioprinted pancreatic tissue used for studies in rats compared to scaled-up tissue for large animals.

FIG. 25C shows viability of bioprinted neonatal porcine islets confirmed up to 14 days post-print.

FIG. 26 depicts a process flow to manufacture implantable lattice structures containing bioprocessed pancreatic islets in materials that protect the allogeneic cells from host immune cell attack.

FIG. 27 depicts a fiber structure with both an inner conformal coating and an outer conformal coating.

### DETAILED DESCRIPTION

The means for suspending of the present disclosure advantageously enables the continuous bioprinting of a cross-linkable fiber such that the resultant structure can be suspended during one or more of printing, patterning and/or post-printing processing. As demonstrated herein, the means and/or a fabrication platform comprising same readily facilitates post-printing modifications as well as the storage and/or transport of a bioprinted fiber structure. In embodiments, the invention facilitates post-printing modifications such as, e.g., coatings that can enhance stability and/or impart anti-FBR properties. In embodiments, the invention enables the production of bioprinted fiber structures with more uniform conformal coatings, including coatings that are free, or substantially free from imperfections that may otherwise contribute to FBR when implanted in a subject. In embodiments, fiber structures generated in accordance with teachings of the present disclosure advantageously exhibit a reduction in FBR when implanted in a subject.

### Definitions

For purposes of interpreting this specification, the following definitions will apply, and whenever appropriate, terms used in the singular will also include the plural and vice versa. In the event that any definition set forth conflicts with any document incorporated herein by reference, the definition set forth below shall control. Unless defined otherwise, all technical and scientific terms used herein have the same meaning as commonly understood by one of ordinary skill in the art to which the disclosure pertains.

The term “hydrogel” as used herein refers to a composition comprising water and a network or lattice of polymer chains that are hydrophilic.

The term “sheath fluid” or “sheath solution” as used herein refers to a fluid that is used, at least in part, to envelope or “sheath” a material as the material is passing through a fluid channel. In some embodiments, a sheath fluid comprises an aqueous solvent, e.g., water or glycerol. In some embodiments, a sheath fluid comprises a chemical cross-linking agent. Non-limiting examples of cross-linking agents include divalent cations (e.g. Ca²⁺, Ba²⁺, Sr²⁺, etc.), thrombin, and pH modifying chemicals, such as sodium bicarbonate.

The terms “segmented/compartmentalized” as used herein refer to a discontinuous nature of a type of material and/or a biological material included in core or shell layer(s) of the fibers disclosed herein, e.g. wherein there are intentional gaps in the deposition of the biological material along a length of the fiber. The spacing (e.g., length) between such segments/compartments may be regular (e.g., an approximately same spacing between regions of biological material), or the spacing may be different.

The term “solid core” as used herein refers to a core of a fiber of the present disclosure that is comprised of a particular material (e.g., hydrogel cross-linkable by a chemical cross-linking agent), such that the core does not comprise a lumen along the entire length of the fiber. The term is not intended to refer to a core that is entirely impenetrable along its length, as solid cores of the present disclosure may enable the passage of particular fluids, molecules and/or ionic species throughout the core.

The term “annular fibers” as used herein refers to fibers that are comprised of a solid core, and one or more shell layers surrounding the solid core.

The term “biocompatible materials” as used herein refers to materials in which biological materials including but not limited to cells can be incorporated into and/or be in contact with said biocompatible materials, and where said biocompatible materials do not exhibit an adverse effect on the ability of the biological materials to carry out one or more functions (e.g., cellular functions including but not limited to secretion of biologically relevant molecular species, agonist/receptor binding, signal transduction, and the like).

The term “immunoprotective” as used herein refers broadly to a design aspect of a fiber of the present disclosure that serves to reduce, prevent or eliminate the host immune response including, e.g., immune cell invasion of the fiber upon implantation of the fiber into a body (e.g., mammalian body).

The term “agent” as used herein refers to any protein, nucleic acid molecule (including chemically modified nucleic acid molecules), antibody, small molecule, organic compound, inorganic compound, or other molecule of interest. An agent can include a biologically relevant agent, a therapeutic agent, a diagnostic agent, a pharmaceutical agent, a chelating agent, etc. A therapeutic or pharmaceutical agent is one that alone or together with an additional compound induces a desired response (such as inducing a therapeutic or prophylactic effect when administered in a manner consistent with the present disclosure to a subject. A biologically relevant agent is one that supports another biological process, for example an agent that supports cell viability.

### Introduction

3D bioprinting is an additive manufacturing process where synthetic fibers, optionally cell laden, are laid down in a layer-by-layer fashion to obtain multi-layer 3D structures. Various types of 3D bioprinting techniques have been developed, including extrusion (Panwar A et al., Molecules. (2016); 21: 685; Sakai S et al., Biofabrication. (2018); 10: 045007; Han H W and Hsu S H, Neural Regener. Res. (2017); 12: 1595), inkjet (Gao G et al., Biotechnol. Lett. (2015); 37: 2349; Gao G and Cui X, Biotechnol. Lett. (2016); 38: 203; Bsoul A et al., Lab Chip. (2016); 16: 3351) laser assisted (Sorkio A et al., Biomaterials. (2018); 171: 57; Pagès E et al., J. Nanotechnol. Eng. Med. (2015); 6: 021006; Catros S et al., In Vivo and In Situ Biofabrication by Laser-Assisted Bioprinting, Elsevier, Winston-Salem, USA. (2015)), and stereolithographic (SLA) (Miri A K et al., Adv. Mater. (2018); 30: 1800242; Wang Z et al., ACS Appl. Mater. Interfaces. (2018); 10; 26859; Wang Z et al., Biofabrication. (2015); 7: 045009) printing methods. Of these, extrusion is one of the most common, whereby bioinks are dispensed through one or more syringes to form layer-by-layer scaffolds from fibers.

Advances have also led to the use of microfluidics-based 3D bioprinting systems (Beyer S T et al., in 2013 Transducers Eurosensors XXVII 17^{th} Int. Conf. Solid-State Sensors, Actuators, Microsystems. IEEE, Piscataway, NJ (2013); pp. 1206-1209; Beyer S T et al., in The 17^{th} Int. Conf. on Miniaturized Systems for Chemistry and Life Sciences. (2013); pp. 176-178). With these systems and techniques, a plurality of materials (e.g., bioink, cross-linker, etc.) flow through microchannels which can allow for precision control of one or more of flow, switching, mixing, and the like. When used with a sheath flow that surrounds at least one inner material, microfluidic bioprinting can reduce shear stress during the printing process. Microfluidics-based 3D bioprinting can also advantageously allow for the intersecting of material flows as they exit independent flow paths into a single flowpath (e.g., dispensing channel), to facilitate the production of structures having a core surrounded by one or more shells.

Many of these bioprinting strategies print fibers directly onto a receiving surface, which can be a confounding factor in terms of post-printing processing steps. For example, a coating cannot be applied to the bottom surface of a bioprinted fiber structure that remains in contact with a receiving surface, and manipulation of the structure may cause it to fall apart or otherwise degrade its integrity. Additionally, printing a fiber structure onto a surface can introduce undesired defects or irregularities stemming from contact with the surface itself.

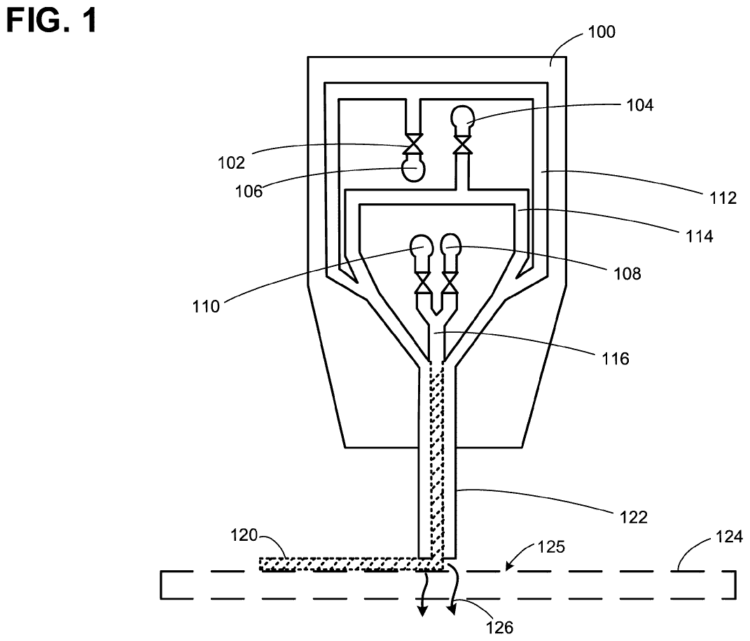

To illustrate the point, FIG. 1 depicts an exemplary microfluidic print head 100 that can be used to print a fiber structure 120. Print head 100 includes a plurality of reservoirs (102, 104, 108, 110), and corresponding valves (depicted simply as “102”). The valves control material flow to respective microfluidic channels, which each converge into a single dispensing channel 122. In this example illustration, microfluidic channel 112 directs a sheath fluid comprising a cross-linker solution toward dispensing channel 122; channel 114 directs a buffer solution towards dispensing channel 122; and channel 116, which receives flow from one or both of reservoir 108 and 110, directs hydrogel material towards dispensing channel 122. Accordingly, reservoir 106 holds sheath fluid, reservoir 104 holds buffer solution, reservoir 108 holds a first hydrogel solution, and reservoir 110 holds a second hydrogel solution. Optionally, one or both of the first and second hydrogel solutions include cells. Flow of each material is controlled by way of valves 102.

Also shown is receiving surface 124, which in the example includes a plurality of pores 125. In an exemplary method, sheath fluid surrounds the hydrogel solution in dispensing channel 122, such that cross-linking of the fiber structure occurs while in the dispensing channel. Any excess sheath fluid flows through the receiving surface 124, as depicted by arrows 126, while fiber structure 120 is deposited on the top of the receiving surface.

FIG. 1 depicts print head 100 depositing a first layer of the fiber structure 120. Additional layers can be added on top of the first layer, for example to form a lattice/grid-like structure. As can be seen, the bottom surface of the fiber structure is in contact with the receiving surface, and accordingly, any procedure to coat the fiber structure with a desired material would not be able to reach the bottom surface of the fiber structure. In some cases, attempting to move and/or manipulate the fiber structure in order to coat the bottom surface may result in degraded fidelity of the fiber structure, due to a lack of effective fiber-to-fiber adhesion. This inability to homogeneously coat (i.e., inability to add a conformal coat) an entirety of an external surface of the fiber structure can render it unsuitable for implantation.

In some cases, the porous nature of the receiving surface can result in imprints in the structure of the resultant fiber along its length. As additional layers are added, the imprints/irregularities can become more pronounced due to the increase in overall weight of bioprinted structure. Even for surfaces that are not porous, the weight of additional layers can compromise structural integrity of a bioprinted fiber structure. Furthermore, in some cases a vacuum may be relied upon to remove excess sheath fluid that flows through the pores 125 of receiving surface 124, and the vacuum can exert a negative force on the fiber in the vicinity of the pores 125, which can further exacerbate the formation of imprints/irregularities. Such imprints/irregularities can be a contributing factor to FBR upon implantation.

### Means for Suspending a Bioprinted Structure

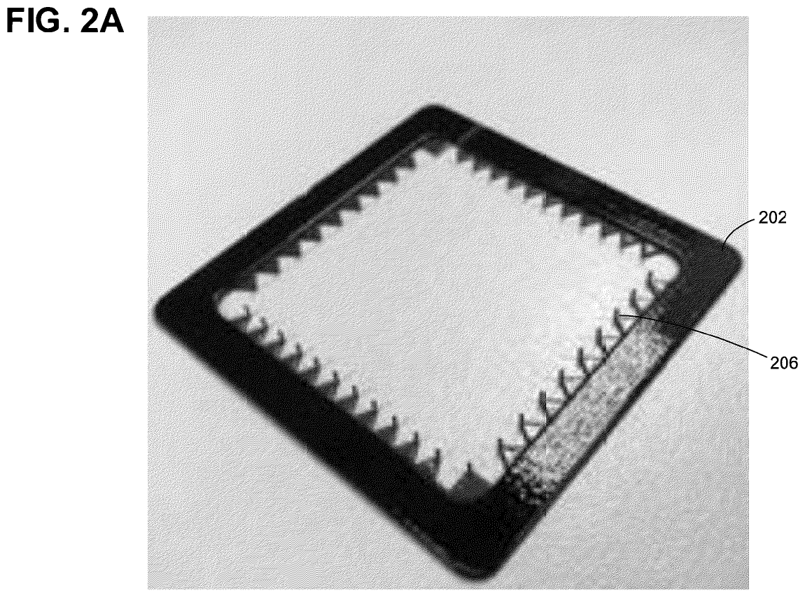

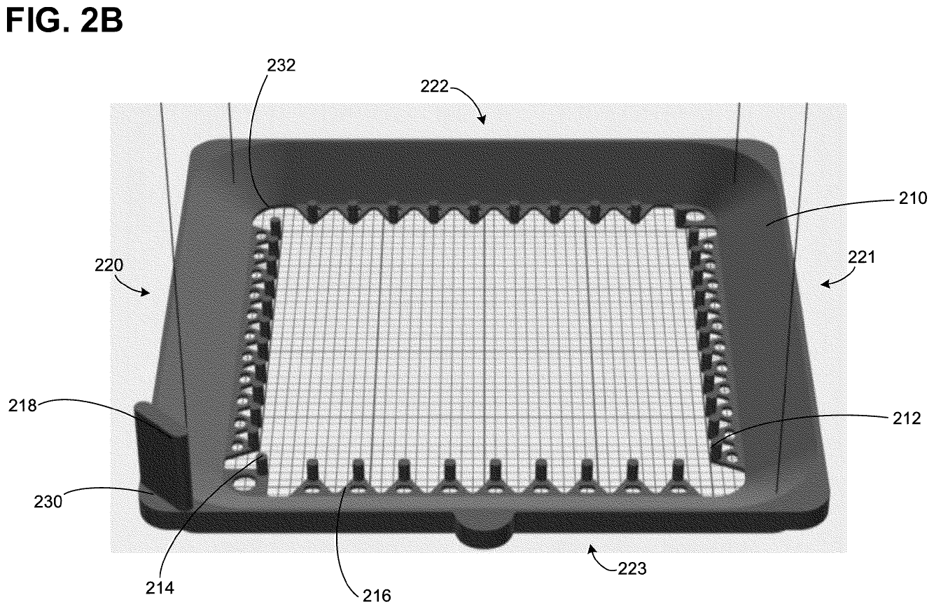

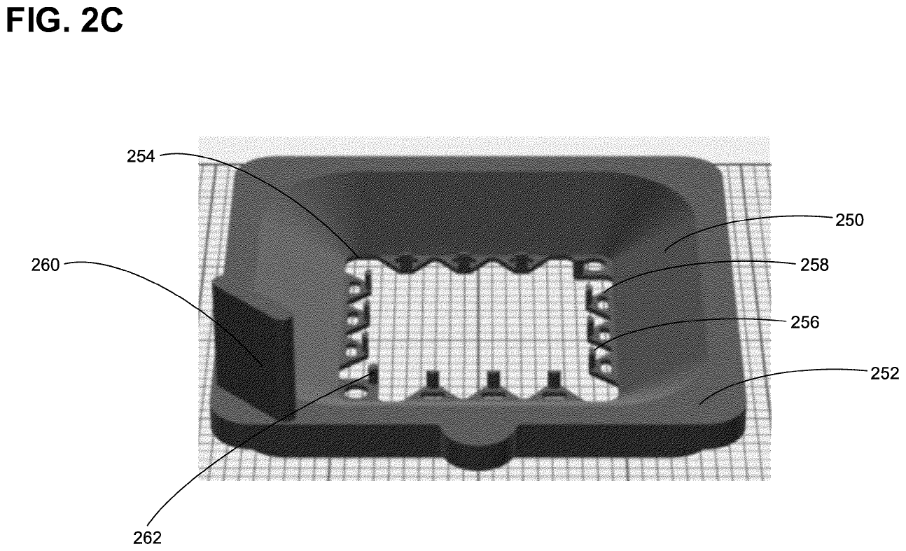

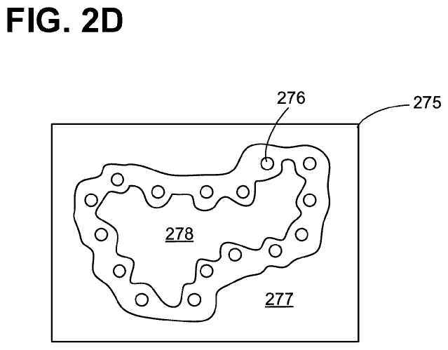

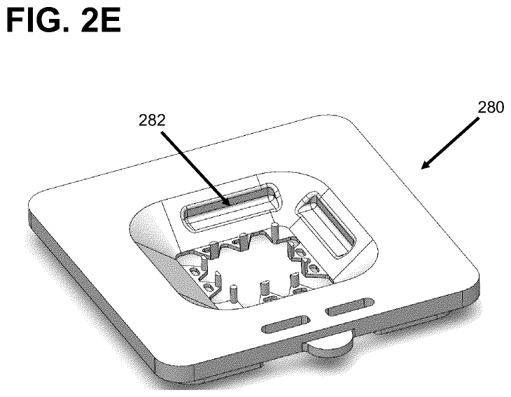

Described herein are means for suspending a fiber structure during printing and/or transport. In this way, a fiber structure can be bioprinted in a manner in which the resultant structure is at least partially suspended during one or more of printing, patterning, and/or post-printing processing. In some examples printing onto a receiving surface can be avoided entirely. Turning to FIG. 2A, depicted is an exemplary means for suspending a fiber structure during printing. In this example embodiment, the means for suspending comprises a frame 202 having a plurality of opposing posts 206. In embodiments, the frame and the posts 206 are comprised of a single fabricated material, although it is within the scope of this disclosure that the posts 206 can be made of a different material than the frame 202. In embodiments, the frame 202 and/or the posts 206 are comprised of e.g., stainless steel, or a dental grade polymer (e.g., polyethylene, polymethyl methacrylate, polycarbonate, polyethylene glycol, polyurethane, hexamethyldisilazane, and the like). In embodiments, the choice of material composition of the posts pertains to minimizing or optimizing a level of adherence of a fiber structure to said posts.

As depicted in FIG. 2A, the empty frame 202 defines a void and comprises a plurality of opposing posts 206 on opposite sides of the frame and extending approximately vertically upward with respect to the frame 202. In the exemplary embodiment shown, the posts 206 are positioned on the interior of said frame 202 and more specifically on projections of the frame 202 extending over the void so as to minimize contact between the fiber and the frame 202 during the printing process. In the embodiment shown at FIG. 2A, frame 206 has a general shape of a square, but other frame shapes are readily contemplated as falling within the scope of this disclosure, including but not limited to rectangular, circular, triangular, hexagonal, and octagonal. In embodiments, the posts can be evenly spaced and on all sides of the frame, but spacing can also be irregular, and/or opposing posts can be positioned on only two sides of the frame, depending on the application. At a minimum, the plurality of posts 206 need to be positioned on opposing sides of the frame and spaced far enough apart to enable a needle or dispensing nozzle to move around/between posts as a fiber structure is being printed.

The number of posts included in a frame for suspending a fiber structure during printing can vary, and can be a function of a number of variables including but not limited to dimensions (e.g., perimeter length) of the corresponding frame, size of dispensing needle/nozzle, desired application, post thickness, post shape (e.g., cylindrical, square, half-cylindrical), etc. For example a frame may comprise at least 10, at least 15, at least 20, at least 25, at least 30, at least 35, at least 40, at least 45, at least 50, or greater than 50 posts, for example 100 posts or more. Post thickness (e.g., diameter in the case of a cylindrical or half-cylindrical post) and post height are additional variables that can be selected depending on the desired application. For example, post height may vary from between about 0.1 mm to about 10 cm, preferably between about 0.2 mm to about 20 mm, for example between about 1 mm and about 4 mm.

Turning to FIG. 2B, depicted is another exemplary embodiment of a frame 210. Frame 210 includes posts 212, along with corner posts 214. Frame 210 has a greater number of posts along side 220 and side 221 (11 posts on each side), and a lesser number of posts (9 posts on each side) along side 222 and side 223. In these aspects, frame 210 is substantially similar to frame 202 (refer to FIG. 2A). In other examples each side may have a same number of posts. Furthermore, although depicted as having two posts in two opposing corners, other embodiments encompass posts in all four corners, just three corners, just one corner, or no corners.

In embodiments, the posts are positioned on frame projections extending internally from the frame into the interior of the frame. In particular with reference to FIB 2B, each post (210 and 214) is positioned on a projection 216 that extends inward from frame 210. In FIG. 2B each projection 216 is triangular, although projections 216 that support posts (e.g., 210 and 214) can be other shapes as well (e.g., rectangular, square, semi-circular). In some embodiments, posts (e.g., 210, 214) can be attached directly to the frame interior (e.g., frame 210) in lieu of projections 216. Frame 210 includes handle 218, which can be used for grasping (e.g., manually or robotically) and manipulating/moving frame 210.

As shown at FIG. 2B, frame 210 has an approximate bowl shape, with handle 218 affixed to upper edge 230 and projections 216 extending from lower edge 232. In embodiments, the overall bowl shape can enable frame 210 to conveniently be placed in a corresponding mounting structure, as will be elaborated in greater detail below.

For reference, another frame 250 is depicted at FIG. 2C. Clearly depicted is upper edge 252 and lower edge 254. Posts 256 each extend from projections 258 extending inward from lower edge 254. Handle 260 extends from upper edge 252. In the example shown at FIG. 2C, each side has a same number of posts, with corner posts 262 occupying two corners of frame 250.

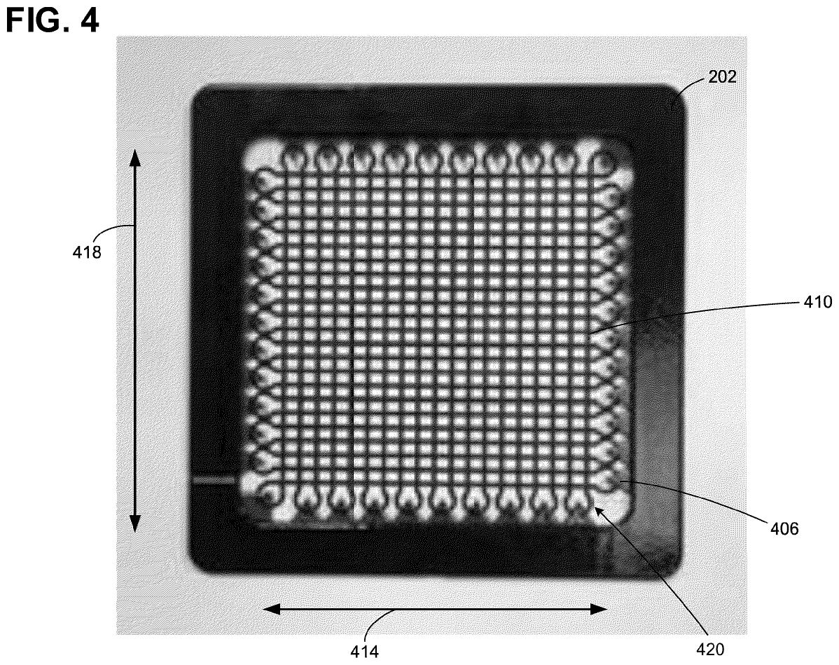

Depending on the frame, bioprinted fiber structures of the present disclosure can have different infill densities. Discussed herein, infill density is expressed as a percentage. For example, a lattice structure having a completely filled fibrous structure (i.e., no spaces) would correspond to an infill density of 100%, whereas a lattice structure that is 90% unoccupied by any fibrous structure would correspond to an infill density of 10%. Related to infill density is fiber to fiber distance. Discussed herein, fiber to fiber distance refers to a distance of empty space between two adjacent fibers in a layer of a lattice where fibers in a same layer are parallel to one another (see e.g., the exemplary fiber structure depicted at FIG. 4).

The frames depicted at FIGS. 2A-2C are exemplary, and other frame designs are within the scope of this disclosure. FIG. 2D depicts another frame 275 where posts 276 are irregularly spaced. Frame 275 includes a common outer envelope 277, and void space 278. Frame 275 is illustrated from a top-down perspective.

In some embodiments, the frame can include a fiber cutting groove positioned such that a cutting implement, such as a scalpel, can be used to cut a portion of the fiber structure such as a long initial fiber that can be “waste.” The fiber cutting groove can permit alignment of the cut without having to remove the fiber structure prior to cutting the “waste” portion of the fiber. An illustrative frame 280 with a fiber cutting groove 282 is shown in FIG. 2E.

Thus, as illustrated with regard to FIGS. 2A-2E, frames in accordance with the present disclosure may be of a particular shape (e.g., square, rectangular, triangular, hexagonal, octagonal, circular, irregular, etc.). In some examples, posts (e. g,, posts 206 at FIG. 2A) may be evenly spaced around the frame, as depicted illustratively at FIGS. 2A-2C. However, in other examples posts (e.g., posts 276 at FIG. 2D) may be irregularly spaced around the frame. In embodiments, the posts are positioned on the interior of said frame, and preferably on frame projections extending internally from the frame into the void formed by the frame so as to minimize contact between the fiber and the frame during the printing process. In embodiments, an overall shape of a frame may be specific for its use in conjunction with a particular mounting bracket, as elaborated in greater detail below. Accordingly, in some embodiments a frame can be used to print a fiber structure comprised of a regular lattice structure. In some embodiments, a frame can be used to print a fiber structure comprised of an irregular lattice structure.

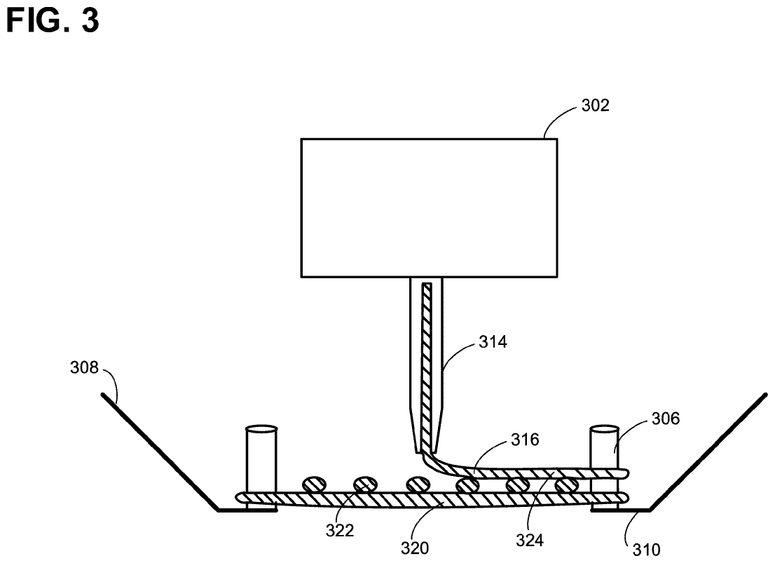

Turning to FIG. 3, depicted is a print head 302, posts 306, frame 308, projections 310, dispensing channel 314, and bioprinted fiber structure 316. As shown, the fiber structure can be printed around posts 306, thus enabling the fiber structure 316 to be at suspended within the frame during printing, patterning and/or post-printing processing. Only two opposing posts (left- and right-side) are shown for illustrative purposes at FIG. 3. In the illustrative example depicted at FIG. 3, fiber structure 316 is a grid/lattice structure comprised of first layer 320, second layer 322, and third layer (being formed) 324. Layer 322 is depicted cross-sectionally, and is formed by sequentially printing around opposite posts of frame 308. The posts (e.g., 306 at FIG. 3) can additionally serve to impart tension to the fiber as it is being printed. This can serve to ensure substantial fiber linearity between posts, and can in some examples assist in maintaining a desired distance between adjacent fiber sections. In some embodiments where the lattice/grid is printed by way of a frame such as that depicted in FIGS. 2A-2C, a distance of empty space between adjacent fibers, referred to herein as fiber to fiber distance, may be between about 1000 μm and 2000 μm, for example between about 1400 μm and about 1600 μm.

Turning to FIG. 4, depicted is frame 202 and posts 206, along with an illustrative fiber structure 410. Fiber structure 410 is shown wrapped around each of opposing posts 206 in a manner that produces a bioprinted lattice structure suspended within the frame. To generate the fiber structure, a first layer may be produced by moving the fiber back and forth in the direction of arrows 414 around consecutive opposing posts 206, and then a second layer may be produced by moving the fiber back and forth in the direction of arrows 418 again around consecutive opposing posts 206, or vice versa. In principle, any number of layers can be added in such fashion. In embodiments, the height of posts 206 can vary as a function of a number of layers desired for a particular structure, where the greater the post height, the greater the potential number of layers. Layer height as discussed herein is a function of fiber diameter, hence for a same overall height of a bioprinted fiber structure, a structure comprised of smaller diameter fibers would have more layers than a structure comprised of larger diameter fibers. As one representative example, a 10 mm tall structure made up of 0.050 mm diameter fibers would have 200 layers.

Preferably, a fiber structure is produced by wrapping a continuous length of a fiber around at least two, three, four, five, six, seven, eight, nine, ten, or more, for example 20 or more, 30 or more, 40 or more, 50 or more, or even 100 or more posts during printing of the fiber structure.

As depicted in FIG. 4, the production of fiber structure 410 in the manner discussed results in loops 420. In embodiments, the loops can be removed from the fiber structure post-printing, or can remain as part of the fiber structure. In embodiments where the loops are cut off, the loops can be cut off while the fiber structure otherwise remains attached to the frame, or the fiber structure can be removed from the frame and then the loops can be cut off.

In embodiments, reliance on a frame as depicted at FIGS. 2A-4 for generating a bioprinted fiber structure enables the entire fiber structure to be manipulated without disturbing the structural integrity of the fiber structure before and/or during intra-layer fiber-fiber adhesion. Accordingly, a bioprinted fiber structure coupled to a frame can be raised above a surface (e.g., receiving surface), and coated in its entirety. In embodiments, removal of a fiber structure (e.g., a fiber structure substantially similar to fiber structure 410) can be accomplished simply by inverting the frame such that gravity acts on the fiber structure to release the fiber structure from the frame. In some additional or alternative embodiments, a force can be applied to an underside of the frame while the frame is inverted or at least partially inverted, to assist in releasing the fiber structure from the frame.

### Fabrication Platforms

Fabrication platforms for producing bioprinted fiber structures as herein disclosed comprise at least the aforementioned means for suspending a bioprinted fiber structure (e.g., frame 202 at FIG. 2A, frame 210 at FIG. 2B, frame 250 at FIG. 2C, frame 275 at FIG. 2D, frame 280 at FIG. 2E) comprising a plurality of posts for securing at least one bioprinted fiber to form said bioprinted fiber structure. For example, in embodiments the means for suspending a bioprinted fiber structure is a standalone device that can be used, e.g., in conjunction with a particular bioprinter system. In other embodiments, one or more additional components can be included as part of a fabrication platform.

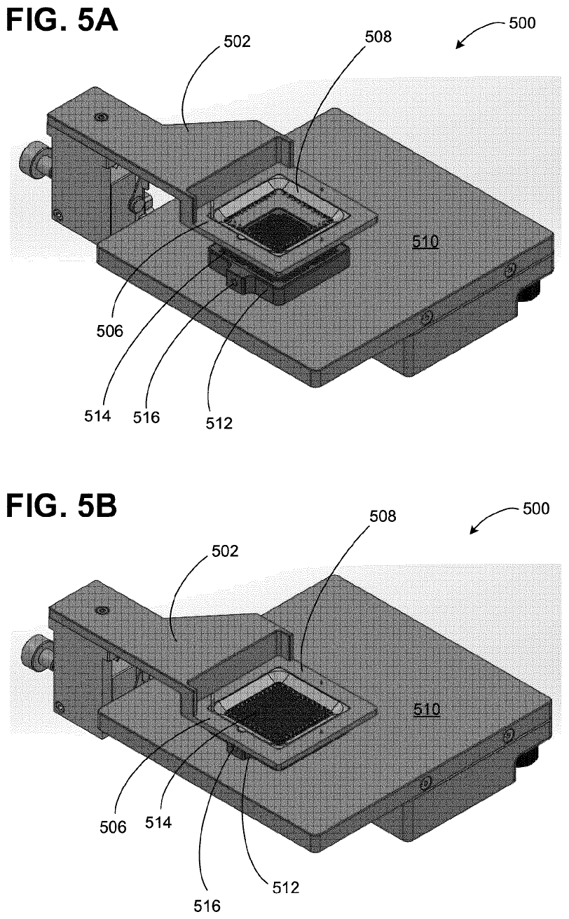

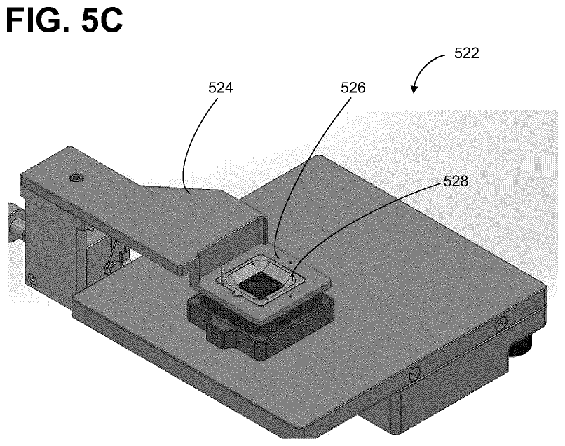

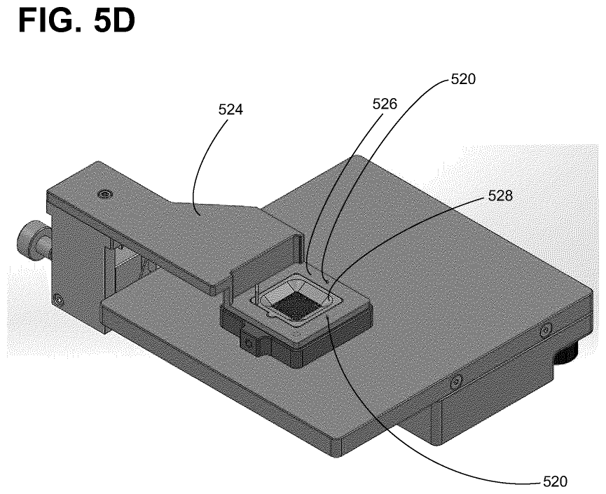

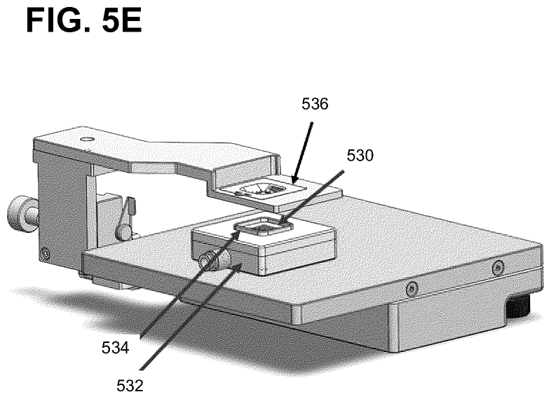

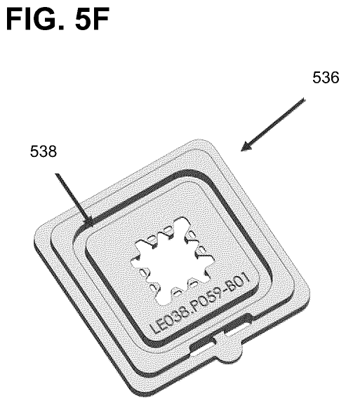

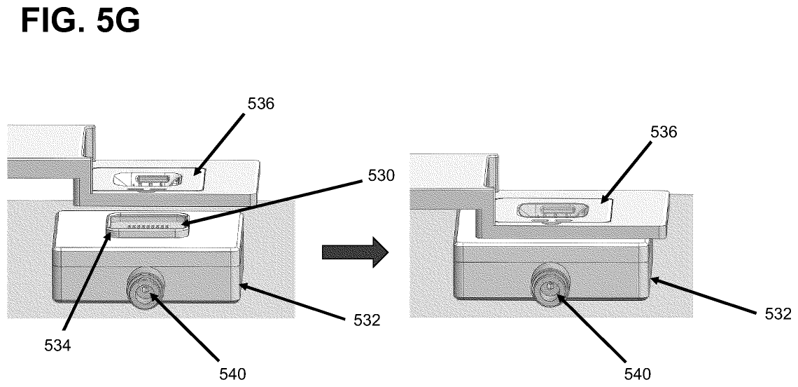

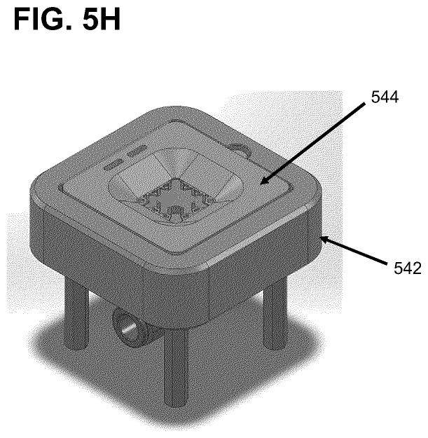

Turning to FIG. 5A, depicted is fabrication platform 500. Fabrication platform 500 includes lifting arm 502, coupled to mounting bracket 506. Mounting bracket 506 is configured to receive frame 508 (e.g., similar or the same as frames depicted in FIGS. 2A-2D). Lifting arm 502, and in turn mounting bracket 506 and frame 508 can be adjusted manually or robotically (i.e., in automated fashion) so as to adjust a height at which frame 508 sits with respect to surface 510. In embodiments, lifting arm 502 is adjustable between a finite number of positions (e.g., 2, 3, 4, 5, 6, 8, 10). In embodiments, lifting arm 502 is adjustable between any number of positions. In either case, with lifting arm 502 set at a desired position, lifting arm 502 can be secured so as to prevent lifting arm from further movement, until further adjustment is desired.

In embodiments, fabrication platform comprises vacuum chuck 512. Vacuum chuck 512 comprises chuck orifice 516, which can be used in conjunction with a vacuum source (e.g., vacuum pump) to draw a vacuum on an interior of vacuum chuck 512. Vacuum chuck 512 can be placed on surface 510, and aligned with frame 508 and mounting bracket 506. In embodiments, a top of vacuum chuck 512 is of an area substantially the same or greater than an area corresponding to the interior of frame 508. In embodiments, the top of vacuum chuck 512 is porous, such that vacuum applied to vacuum chuck 512 by way of chuck orifice 516 draws air through the top of vacuum chuck 512 and into an interior space of vacuum chuck 512. In embodiments, the top of vacuum chuck 512 is uniformly porous throughout said top.

In embodiments, a mesh 514 can be placed on top of vacuum chuck 512. Mesh 514 can be reusable, or disposable. Preferably, mesh 514 is comprised of a non-adherent, non-reactive, synthetic material that can in some examples act as a passive support for a bioprinted fiber structure. Examples include but are not limited to nylon, polyethylene, polyethylene terephthalate, steel, glass, PEEK (poly-ether-ether keytone), PTFE (poly-tetrafluoroethylene), cellulose, and the like.

In embodiments, via lifting arm 502, frame 508 can be lifted away from surface 510, so as to enable placement of, e.g., vacuum chuck 512 (and optionally, mesh 514) at a desired position with respect to frame 508. Then, via lifting arm 502, frame 508 can be lowered in the direction of surface 510 so as to position frame 508 at a desired position with respect to vacuum chuck 512 (and optionally, mesh 514), as depicted illustratively at FIG. 5B. It may be understood that the vacuum drawn by way of vacuum chuck 512 may serve to remove excess fluid (e.g., excess sheath fluid and/or excess buffer solution) that may otherwise pool on mesh 514, top of vacuum chuck 512, and/or surface of bioprinted fiber structure. Accordingly, in the embodiment depicted at FIGS. 5A-5B, vacuum chuck 512 comprises a fluid removal component.

The mounting bracket (e.g., mounting bracket 506) and, optionally, lifting arm (e.g., lifting arm 502) may be designed to accommodate a frame with particular dimensions. In some embodiments, the mounting bracket and the lifting arm are a single unit, while in other embodiments, the mounting bracket may be detachably coupled to the lifting arm.

Accordingly, for reference FIG. 5C depicts another exemplary embodiment of a fabrication platform 522 with lifting arm 524 in a first, upper position, and FIG. 5D depicts fabrication platform 522 with lifting arm 524 in a second, lower position. Mounting bracket 526 as shown at FIGS. 5C-5D is designed to accommodate frame 528. Fabrication platform 522 also can include vacuum chuck and mesh (non-numbered) as discussed above with regard to FIGS. 5A-5B. As visually apparent, fabrication platform 522 accommodates a smaller frame than fabrication platform 500. In some embodiments, mounting bracket(s) may detachably couple to the lifting arm of particular fabrication platforms, in order to accommodate different frame configurations and/or dimensions.

In embodiments, one or more homing posts 520 are included as part of mounting bracket 526, as shown in FIG. 5D. Homing posts 520 can be used to align e.g., a print head of a bioprinting system as herein disclosed. Homing posts 520 function as reference coordinates, which for example can assist a bioprinter system in finding and setting a “HOME” position for printing a 3D fiber structure. For example, the HOME position may correspond to known x, y, z coordinates relative to homing posts 520. In this way, a bioprinting system can register dimensions of a particular frame, and coordinate movement of a print head as a function of the reference coordinates.

In embodiments, a continuous length of at least one bioprinted fiber is printed around at least two, three, four, five, six, seven, eight, nine, ten or more, for example 20 or more, 30 or more, 40 or more, 50 or more, or even 100 or more of said posts of a frame (e.g., frame 508) during a 3D bioprinting process via the use of such a fabrication platform as disclosed. Such fabrication platforms can comprise part of bioprinting systems as herein disclosed.

In embodiments, mesh (e.g., mesh 514 at FIGS. 5A-5B) and/or top of a vacuum chuck (e.g., vacuum chuck 512) can comprise a receiving surface for a bioprinted fiber structure printed by way of posts corresponding to a frame (e.g., frame 508). In embodiments, a receiving surface can comprise, for example, a surface (e.g., surface 510) that does not include the vacuum chuck (e.g., vacuum chuck 512), but can optionally include some other additional or alternative fluid removal component.

Accordingly, in embodiments, a receiving surface is flat or substantially flat. In embodiments, a receiving surface is solid. In embodiments, a receiving surface is porous. In embodiments, a fiber structure is printed onto a receiving surface (e.g., mesh 514), by way of the frame (e.g., frame 508), and then the frame and corresponding fiber structure is suspended above the receiving surface for further processing, for example application of one or more coats to the fiber structure.

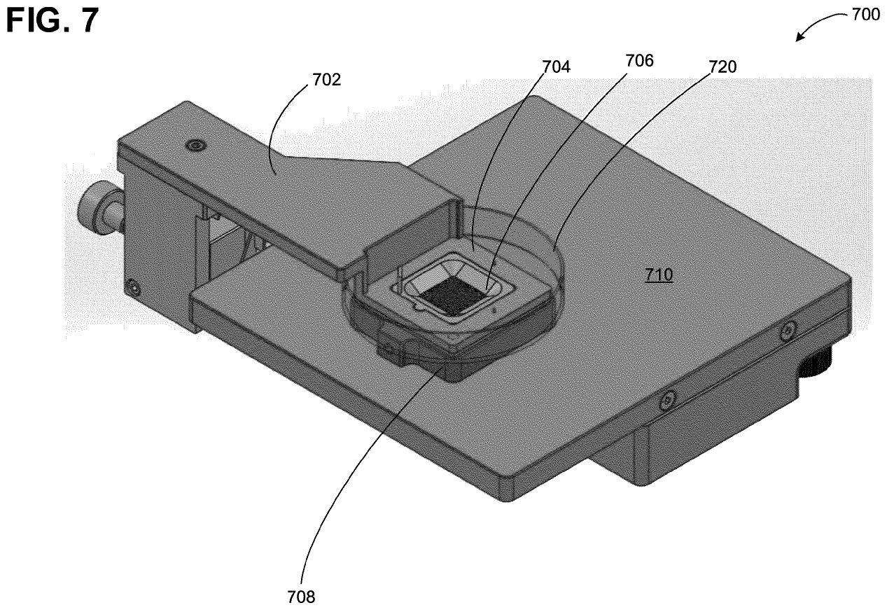

In some embodiments, a fabrication platform can comprise a vessel (see, e.g. FIG. 7). In embodiments, the vessel comprises a container into which at least a portion of the fabrication platform, for example at least a means for suspending a bioprinted fiber structure (e.g., frame 508 at FIG. 5A), optionally coupled to a mounting bracket (e.g., mounting bracket 506 at FIG. 5A), can be placed therein. For example, the container can comprise a bath used during one or more of printing, patterning and/or modifying the bioprinted fiber post-printing. In embodiments, the vessel is sized such that a liquid solution held by the vessel can completely submerge at least a means for suspending a bioprinted fiber structure (e.g., frame 508 at FIG. 5A), optionally coupled to a mounting bracket (e.g., mounting bracket 506 at FIG. 5A). In embodiments, such a vessel may be used for submerge bioprinting, discussed below.

In embodiments, an integrated container 530 can be formed directly on the top surface of the vacuum chuck 532 by walls 534 defining the perimeter of the container, as shown in FIG. 5E. The walled area creates an integrated container that can retain liquids, e.g. a cross-linker solution, a wash solution, a storage solution, a dip-coat solution, etc., onto which the frame 536 can be placed. In embodiments, the walls are configured to interface with a mating groove 538 on the underside of the frame 536 as shown in FIG. 5F. Preferably, the depth of the groove is such that the volume of liquid within the container can sit just above the post height of the frame. By having this walled volume of liquid, the user can precisely dispense a specific volume of liquid onto the frame.

This configuration can enable suspending the fiber structure within liquid in the container without having the fiber structure contact the vacuum chuck and/or mesh. In an exemplary embodiment, the walled volume of liquid may be sufficient for coating at least an underside of the fiber structure upon placement of the frame on the integrated container, and an additional volume of liquid may optionally be applied on top, as illustrated in FIG. 5G. In embodiments, the vacuum chuck can be operatively connected to a valve (540) that can be operated to allow liquid to flow out from the vessel under vacuum or prevent the flow of liquid to maintain a level of liquid in the vessel.

In embodiments, the frame or a portion thereof can be coated with a hydrophobic coating, such as Parylene-C, to prevent leaking. In embodiments, the vacuum chuck (542) with integrated container can be elevated on supports and used as a standalone dip-coat station for the frame (544), as shown in FIG. 5H.

The fabrication platforms depicted at FIGS. 5A-5D are illustrative, and other variations are within the scope of this disclosure. Turning to FIG. 5I, depicted is another example of a fabrication platform 570 that may comprise part of a bioprinting system as herein disclosed. Fabrication platform 570 comprises frame 571, vacuum chuck 572, and motionless dock 574, which also includes frame dock 575. Coupled to frame 571 are mounting brackets 577. Vacuum chuck 572 includes pegs 576. Also shown is stage 573. A bioprinting system may move stage 573 along x, y and z planes (see Cartesian coordinate system 579). Vacuum chuck 572 may rest or otherwise be secured to stage 573. Frame dock 575 may removably couple to frame 571, such that frame dock and hold in position frame 571 when frame 571 is not coupled to vacuum chuck 572 by way of mounting brackets 577. In this example illustration, pegs 576 coupled to vacuum chuck 572 are positioned in the uppermost of grooves 578, such that frame 571 is positioned directly atop vacuum chuck 572.

Turning to FIG. 5J, illustrated is fabrication platform 570 in which frame 571 is coupled to frame dock 575, and stage 573 has moved away from frame 571. FIG. 5K depicts fabrication platform 570 in which stage 573 has moved in a manner so as to position pegs 576 in the lowermost grooves of mounting brackets 577. In doing so, frame 571 is effectively suspended (Δh) above vacuum chuck 572. This can enable a bioprinted fiber structure to be fully suspended for post-printing processing, such as coating an entirety of the bioprinted fiber structure. In regards to FIGS. 5I-5K, movement of the stage 573 may be automated, such that a control system of the bioprinting system can effectively position the frame at desired distances from the vacuum chuck (or other receiving surface) via programmed control sequences.

### Submerge Bioprinting

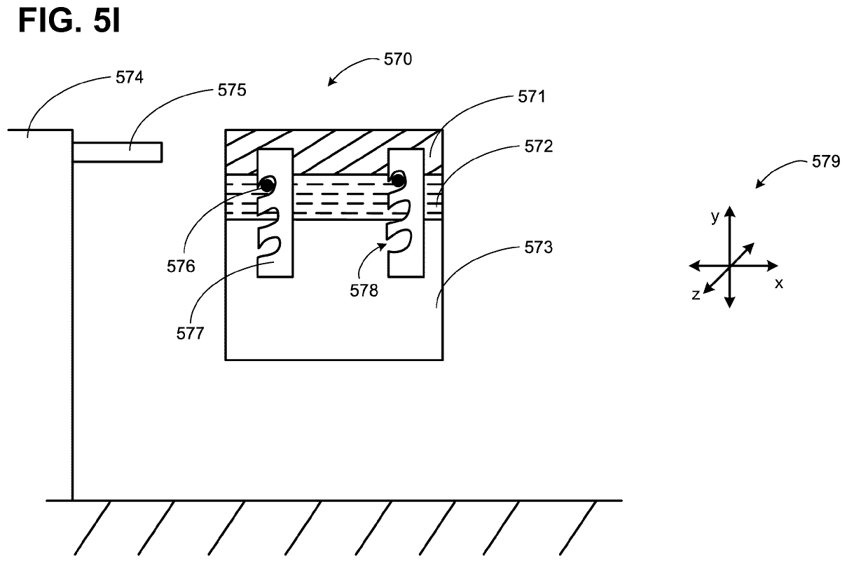

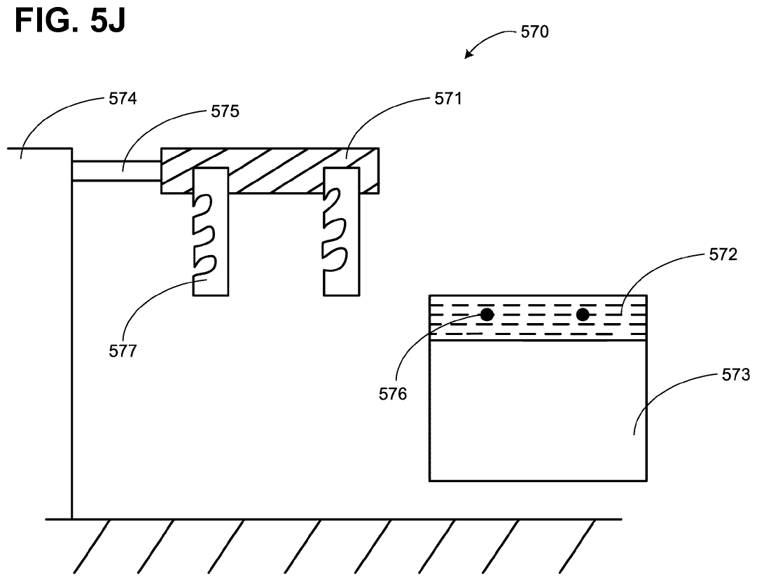

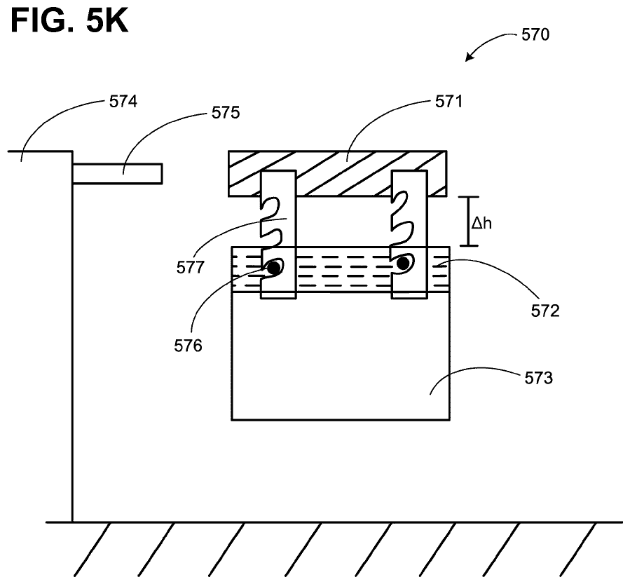

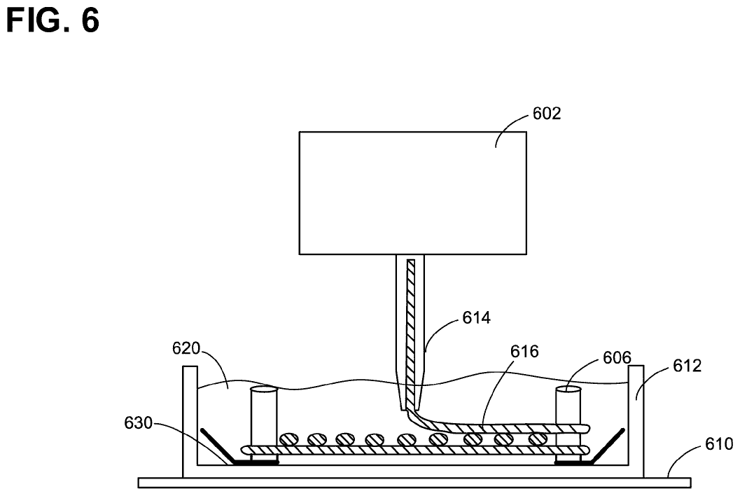

In embodiments, a fiber structure can be printed into a bath that contains a cross-linker solution, where said bath includes a means for suspending the fiber during printing. FIG. 6 shows an exemplary depiction of such a process. As illustrated, FIG. 6 includes print head 602, posts 606 (only two of which are shown for clarity) included as part of the means for suspending a bioprinted fiber structure, a surface 610, and a container 612 which sits atop surface 610 and which holds cross-linker solution 620. In some embodiments, as will be discussed in greater detail below, surface 610 may comprise a top surface of a vacuum chuck (e.g., vacuum chuck 512 at FIGS. 5A-5B). In this way, the bioprinted fiber structure 616 emanating from dispensing channel 614 can be cross-linked effectively on all sides immediately after/during its being dispensed. Further, the fiber can be suspended above the bottom surface 630, entirely or at least partially, for at least a portion of the printing process. Printing into a bath containing a cross-linker solution can reduce gravitational effects on a bioprinted fiber structure that is fully or at least partially suspended, which can serve to assist in avoiding weight-induced distortions and maintaining structural integrity of the bioprinted structure. In additional or alternative examples, printing of a fully or at least partially suspended fiber structure in a bath containing a cross-linker solution can prevent the introduction imprints/irregularities into the bioprinted fiber which may contribute to FBR when implanted into a subject.

In embodiments, following generation of the desired bioprinted fiber structure, the cross-linking solution can be removed. In some embodiments, removal of the cross-linking solution includes aspirating or otherwise draining (e.g., via an appropriately positioned removable plug) said solution out of the container. In such an embodiment, one or more additional fluids can then be added to the container for further processing and/or storage purposes. For example, one or more wash steps may be conducted, wherein following removal of the cross-linking solution, a wash buffer is added to the container and then removed, where this process can repeat any number of times. In some additional or alternative embodiments, the means for suspending the bioprinted tissue structure can be removed from the cross-linking solution with the bioprinted fiber structure attached thereto. In such an example, the process of removing the means for suspending the bioprinted structure can be done manually, or can be automated. In embodiments, once the means for suspending the bioprinted structure has been removed from the cross-linking solution, the attached bioprinted fiber structure can optionally be further processed. In one embodiment, the means and attached bioprinted fiber structure can be placed in another container that holds another solution for further processing and/or for storage purposes. Other additional or alternative processing steps are discussed in greater detail below. Importantly, the means for suspending the bioprinted fiber structure enables the fiber structure to be moved without disrupting its structural integrity.

Turning to FIG. 7A, depicted is an illustration of fabrication platform 700 comprising lifting arm 702, mounting bracket 704, frame 706, vacuum chuck 708, surface 710, and vessel 720. In the embodiment depicted at FIG. 7A, vessel 720 sits atop vacuum chuck 708. In this way, mounting bracket 704 and frame 706 can be lowered into a solution (not specifically depicted) contained within vessel 720. In some embodiments, the solution can comprise a cross-linker solution as discussed, and optionally, a fiber structure can be printed onto the frame 706 while said frame is submerged in the cross-linker solution. In additional or alternative embodiments, the bioprinted fiber structure need not necessarily be printed into a solution, but the bioprinted fiber structure attached to the frame can be lowered into a solution contained within vessel post-printing. The solution can be, for example, a cross-linker solution, a wash solution, a storage solution, or in some embodiments, a dip-coat solution (discussed in further detail below).

In some embodiments, by placing the vessel (e.g., vessel 720 at FIG. 7A) atop the vacuum chuck (e.g., vacuum chuck 708 at FIG. 7A), the frame (e.g., 706 at FIG. 7A) can be readily lifted out of a solution contained within the vessel, the vessel can then be removed from the top of the vacuum chuck, and the frame can then be lowered into close proximity with the top of the vacuum chuck so as to rely on the associated vacuum for removal of excess fluid associated with one or more of the bioprinted fiber structure, frame, and/or mounting bracket. Such a process can be repeated any number of times as so desired.

### Conformal Coating

Discussed herein, a coating process can add one or more additional outer layer(s) to a bioprinted fiber structure. Advantageously, the subject invention enables the entirety of a printed fiber structure, including a bottom surface thereof, to be uniformly coated one or more times. In embodiments, these additional one or more outer layer(s) can impart anti-FBR properties and/or increased stability to a bioprinted fiber structure.

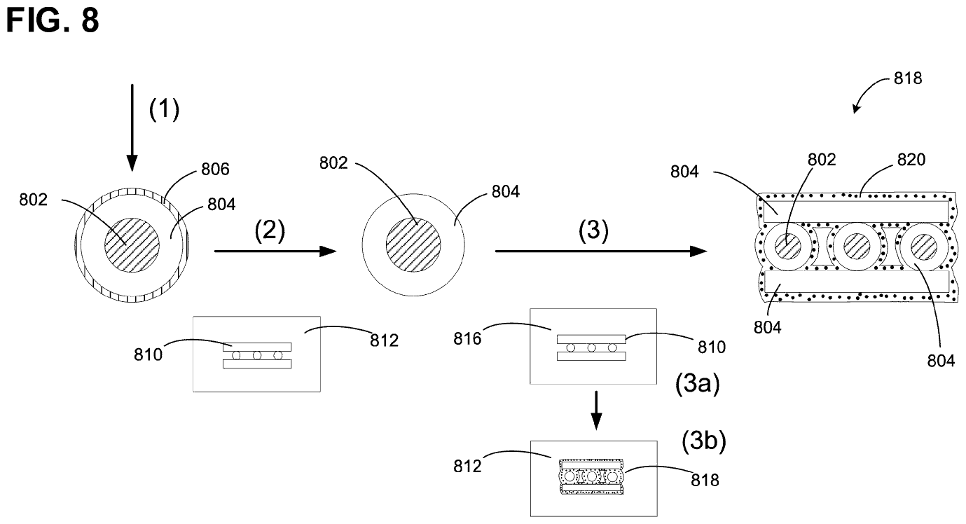

Turning now to FIG. 8, depicted is an exemplary process flow for adding a conformal coat to a bioprinted fiber structure of the present disclosure. In the exemplary process flow of FIG. 8, a bioprinted fiber that forms the fiber structure is produced in a manner whereby the fiber is exposed to a sheath fluid containing cross-linker during printing. It is to be understood that the bioprinted fiber structure discussed in the context of FIG. 8 may comprise a grid/lattice structure produced via a means for suspending a bioprinted fiber structure (e.g., a frame as depicted at FIGS. 2A-2D) as herein disclosed.

Step (1) at FIG. 8 comprises printing the fiber structure. In this example, the fiber that makes up the fiber structure comprises a core 802, and external shell layer 804 surrounding said core, which in turn is surrounded by sheath fluid 806 comprising a cross-linker during printing. In embodiments, the external shell layer 804, and optionally the core 802, comprises a cross-linkable material. In embodiments, the core 802 is solid, optionally further comprising at least one biological material (e.g., cells). Although depicted as one external shell layer, bioprinted fibers comprising more than one shell layer are within the scope of this disclosure. As the fiber structure is printed onto the means for suspending, the sheath fluid is removed, for example via flowing through a porous receiving surface (not shown) as discussed above. Following printing, optional step (2) includes submerging the entire fiber structure 810 in cross-linking solution 812 to facilitate/continue uniform cross-linking of the entire printed fiber structure. While not explicitly illustrated, in other additional or alternative embodiments, the entire fiber structure can be washed with cross-linking solution, for example by dispensing cross-linking solution onto the fiber structure while remaining suspended via the means for suspending (e.g., a frame as depicted at FIGS. 2A-2D).

Step (3) is divided into two sub-steps (3a) and (3b). Step (3a) includes coating the entire fiber structure 810 with coating solution 816. Such coating may comprise, for example, dispensing coating solution 816 onto the fiber structure while the fiber structure is suspended via the means for suspending, and/or submerging the fiber structure in the coating solution 816. In embodiments, the coating solution 816 comprises a cross-linkable material (e.g., alginate). In embodiments, the coating solution 816 comprises a material that is the same as the material comprising the external shell layer 804 of the fiber structure 810 being coated. In embodiments, the coating solution 816 comprises a material that is different than the material comprising the external shell layer 804 of the fiber structure 810 being coated. At step (3a), residual cross-linker (e.g., Ca²⁺) associated with the fiber structure 810 contributes to initial cross-linking of the material in the coating solution 816 with the material comprising the external shell layer 804. Following conformal coating of the fiber structure 810, step (3b) includes submerging (or otherwise washing) the coated fiber structure 818 in/with cross-linking solution 812. In this way, a conformal coat layer 820 is added uniformly to an entirety of the fiber structure as shown.

In embodiments, more than one coating can be sequentially added to a fiber structure. For example, it is within the scope of this disclosure that two, three, four, or more conformal coating layers be sequentially added to a bioprinted fiber structure. Via the use of the subject invention the fiber structure can be suspended during application of each conformal coating layer, such that the entirety of the outer surface of the resultant structure can be coated any number of times. In embodiments, thickness of coating layers can be determined visually, for example using microscopy.

In embodiments, a Ca²⁺ chelator may be applied just prior to step (3a) to remove some amount of Ca²⁺ from the surface of an fiber structure prior to application of the conformal coating, in order to improve adhesion between the fiber structure and the coating layer. Preferred examples of calcium chelators include but are not limited to BAPTA, EDTA, trisodium citrate, and derivatives or analogs thereof. A similar process may be used for sequentially adding multiple conformal coatings to a bioprinted fiber structure.

In embodiments, the conformal coating may be selected in terms of material composition to impart particular properties. Different coating layers may, in examples, be comprised of different materials and/or have different physical properties (e.g., differing levels of hardness). As an illustrative example, a first conformal coating and a second conformal coating may each be comprised of alginate, but at differing percentages. For example, the first conformal coating may be comprised of a higher percentage alginate (e.g., about 2%), whereas the second coating may be comprised of a lower percentage alginate (e.g., about 0.5%). Without being bound by theory, such an approach may impart increased stability due to the higher percentage of alginate in the first coating, and simultaneously impart anti-FBR properties due to the lower alginate percentage in the second coating (Doloff et al. Nat. Biomed Eng. (2021); 5(10): 1115-1130).

Thus, the conformal coatings enabled herein can be advantageously used to impart stability and/or impart anti-FBR properties to a tissue fiber structure. With the present invention, no part of the bioprinted tissue fiber structure will lack the particular properties imparted to it by the one or more coating layers.

In embodiments, a coating layer (e.g., 820) is comprised of a hydrogel material, for example a hydrogel material comprising one or more of alginate, chitosan, GEL-MA, poly(ethylene glycol) (PEG), poly-L-lysine (PLL), triazole, and the like. In some examples, a coating layer is comprised of a functionalized alginate, i.e., an alginate that is chemically modified to include one or more properties, including but not limited to immunoprotective properties that are advantageous in the manufacture of fiber structures of the present disclosure. Examples of functionalized alginates include but are not limited to methacrylated alginate, alginate furan, alginate thiol, alginate maleimide, and covalent click alginates (e.g., alginate blended with [2-(Methacryloyloxy)ethyl]dimethyl-(3-sulfopropyl)ammonium hydroxide (DMAPS)-aldehyde (DMAPS-Ald) and/or [2-(Methacryloyloxy)ethyl]dimethyl-(3-sulfopropyl)ammonium hydroxide (DMAPS)-hydrazide (DMAPS-Hzd)) (see U.S. Provisional Patent Application No. 63/192,552).

In embodiments, the coating solution comprises at least one cross linkable material including but not limited to hydrogels such as alginate, zwitterionic alginate, sulfobetaine methacrylate (SBMA), chitosan, poly(ethylene glycol) diacrylate (PEGDA), poly(ethylene glycol)-tetra-acrylate (PEGTA), Hyaluronic acid (HA), hyaluronic acid methacryloyl (HAMA), collagen, methacrylated collagen (ColMA), gelatin, gelatin methacryloyl (gelMA), agarose, gellan, fibrin (fibrinogen), poly (vinyl alcohol) (PVA), and the like, or any combination thereof.

### Input Materials

Aspects of the invention include input materials that can be used for printing fiber structures for advantageous use as biomaterials. “Biomaterial” as used herein refers to a natural or synthetic substance that is useful for constructing or replacing tissue, e.g. human tissue with or without living cells. In the field of bioprinting, the term “biomaterial” is often synonymous with the term “bioink.”

An input material will generally comprise at least one cross-linkable material, e.g., hydrogels including but not limited to, alginate, chitosan, PEGDA, PEGTA, Hyaluronic acid (HA), HAMA, collagen, CollMA, gelatin, gelMA, agarose, gellan, fibrin (fibrinogen), PVA, and the like, or any combination thereof, as well as non-hydrogels including but not limited to, PCL, poly-(d,l-lactic acid-co-glycolic acid) (PLGA), poly(lactic acid) (PLA), and the like, or any combination thereof. In preferred embodiments an input material comprises at least one hydrogel. Non-limiting examples of hydrogels include alginate, agarose, collagen, fibrinogen, gelatin, chitosan, hyaluronic acid-based gels, or any combination thereof. A variety of synthetic hydrogels are known and can be used in embodiments of the systems and methods provided herein. For example, in some embodiments, one or more hydrogels form at least part of the structural basis for three-dimensional structures that are printed. In some embodiments, a hydrogel has the capacity to support growth and/or proliferation of one or more cell types, which may be dispersed within the hydrogel or added to the hydrogel after it has been printed in a three dimensional configuration.

In embodiments, a hydrogel is cross-linkable by a chemical cross-linking agent. For example, a hydrogel comprising alginate may be cross-linkable in the presence of a divalent cation such as calcium chloride (CaCl₂), a hydrogel containing chitosan may be cross-linked using a polyvalent anion such as sodium tripolyphosphate (STP), a hydrogel comprising fibrinogen may be cross-linkable in the presence of an enzyme such as thrombin, and a hydrogel comprising collagen, gelatin, agarose or chitosan may be cross-linkable in the presence of heat or a basic solution.

In embodiments hydrogel fibers may be generated through a precipitation reaction achieved via solvent extraction from the input material upon exposure to a cross-linker material that is miscible with the input material. Non-limiting examples of input materials that form fibers via a precipitation reaction include collagen and polylactic acid (PLA). Non-limiting examples of cross-linking materials that enable precipitation-mediated hydrogel fiber formation include polyethylene glycol (PEG) and alginate. Cross-linking of the hydrogel will increase the hardness of the hydrogel, in some embodiments allowing formation of a solidified hydrogel.

In some embodiments, a hydrogel comprises alginate. Alginate forms solidified colloidal gels (high water content gels, or hydrogels) when contacted with di valent cations. Any suitable divalent cation can be used to form a solidified hydrogel with an input material that comprises alginate. In the alginate ion affinity series Cd²⁺>Ba²⁺>Cu²⁺>Ca²⁺>Ni²⁺>Co²⁺>Mn²⁺, Ca²⁺ is the best characterized and most used to form alginate gels (Ouwerx, C. et al., Polymer Gels and Networks, 1998, 6(5):393-408). Studies indicate that Ca-alginate gels form via a cooperative binding of Ca²⁺ ions by poly G blocks on adjacent polymer chains, the so-called “egg-box” model (ISP Alginates, Section 3: Algin-Manufacture and Structure, in Alginates: Products for Scientific Water Control, 2000, International Specialty Products: San Diego, pp. 4-7). G-rich alginates tend to form thermally stable, strong yet brittle Ca-gels, while M-rich alginates tend to form less thermally stable, weaker but more elastic gels. In some embodiments, a hydrogel comprises a depolymerized alginate.

In some embodiments, a hydrogel is cross-linkable using a free-radical polymerization reaction to generate covalent bonds between molecules. Free radicals can be generated by exposing a photoinitiator to light (often ultraviolet), or by exposing the hydrogel precursor to a chemical source of free radicals such as ammonium peroxodisulfate (APS) or potassium peroxodisulfate (KPS) in combination with N,N,N,N-Tetramethylethylenediamine (TEMED) as the initiator and catalyst respectively. Non-limiting examples of photo cross-linkable hydrogels include: methacrylated hydrogels, such as hyaluronic acid methacrylate (HAMA), gelatin methacrylate (GEL-MA) or polyethylene (glycol) acrylate-based (PEG-Acylate) hydrogels, which are used in cell biology due to their inertness to cells. Polyethylene glycol diacrylate (PEG-DA) is commonly used as scaffold in tissue engineering, since polymerization occurs rapidly at room temperature and requires low energy input, has high water content, is elastic, and can be customized to include a variety of biological molecules.

In embodiments, an input material comprises a non-biodegradable polymer. In examples the input material may be a synthetic polymer, for example polyvinyl acetate (PVA). In embodiments, an input material may comprise hyaluronic acid (HA).

In some embodiments, a hydrogel comprises a chemically modified alginate. In examples, the chemically modified alginate comprises alginate functionalized with methacrylate groups, referred to herein as “Alg-MA.” In some embodiments, the Alg-MA can be used in an immunoprotective shell layer via blending with zwitterionic alginate, referred to herein as “Alg-zw.” Because of a dual cross-linking capability of Alg-MA, in embodiments the Alg-MA may be first printed with Alg-zw via physical cross-linking. Upon printing the fibers can then be further irradiated to induce covalent cross-linking across fibers thus resulting in F-F adhesion. In some embodiments, the chemically modified alginate may comprise thiolated alginate.

In some embodiments, one or more synthetic components may be added into hydrogel materials. Synthetic components may be useful in increasing fiber-to-fiber adhesion and/or in vivo stability. In examples, a material may comprise an acrylated zwitterionic monomer (e.g., sulfobetaine methacrylate (SBMA) and a cross-linker (e.g., poly(ethylene glycol) diacrylate (PEGDA). In such an example, photomediated cross-linking of the zwitterionic monomer with PEGDA may render the resultant cross-linked polymer matrix superhydrophilic, and hence, less prone to foreign body response (FBR) (see U.S. Provisional Patent Application No. 63/192,552, the contents of which is expressly incorporated by reference herein in its entirety).

In some embodiments, hydrogel materials may be cross-linked via click chemistry. For example, copolymers comprising a zwitterionic monomer and aldehyde motifs (e.g., [2-(Methacryloyloxy)ethyl]dimethyl-(3-sulfopropyl)ammonium hydroxide (DMAPS)-aldehyde, referred to herein as “DMAPS-Ald”), and zwitterionic monomer and hydrazide motifs (e.g., DMAPS-hydrazide, referred to herein as “DMAPS-Hzd”), may be used (see U.S. Provisional Patent Application No. 63/192,552). Aldehyde reacts readily with hydrazide, forming covalently cross-linked hydrogels. Because of the presence of zwitterionic monomer in the polymer backbone, these polymers may exhibit low protein binding properties. In embodiments, one of these polymers may be blended with alginate in a shell. Following printing, the structure may be submersed in a solution containing the counter component that will in turn result in a covalently cross-linked bridge between fibers leading to F-F adhesion.

In embodiments, an input material comprises microparticles, “Microparticles” as used herein refers to immiscible particles in the range of about 0.1 um to about 100 um that are typically composed of a polymer, a metal, or other inorganic material. They can be symmetrical (e.g. spherical, cubic, etc) although this is not a requirement. Microparticles having an aspect ratio of 2:1 or greater may be considered a microrod or microfibre.

Input materials in accordance with embodiments herein can comprise any of a wide variety of natural or synthetic polymers that support the viability of living cells, including, e.g., alginate, laminin, fibrin, hyaluronic acid, poly(ethylene) glycol based gels, gelatin, chitosan, agarose, or combinations thereof. In some embodiments, the subject bioink compositions are physiologically compatible, i.e., conducive to cell growth, differentiation and communication. In certain embodiments, an input material comprises one or more physiological matrix materials, or a combination thereof. By “physiological matrix material” is meant a biological material found in a native mammalian tissue. Non-limiting examples of such physiological matrix materials include: fibronectin, thrombospondin, glycosaminoglycans (GAG) (e.g., hyaluronic acid, chondroitin-6-sulfate, dermatan sulfate, chondroitin-4-sulfate, or keratin sulfate), deoxyribonucleic acid (DNA), adhesion glycoproteins, and collagen (e.g., collagen I, collagen II, collagen III, collagen IV, collagen V, collagen VI, or collagen XVIII).

Collagen gives most tissues tensile strength, and multiple collagen fibrils approximately 100 nm in diameter combine to generate strong coiled-coil fibers of approximately 10 μm in diameter. Biomechanical function of certain tissue constructs is conferred via collagen fiber alignment in an oriented manner. In some embodiments, an input material comprises collagen fibrils. An input material comprising collagen fibrils can be used to create a fiber structure that is formed into a tissue construct. By modulating the diameter of the fiber structure, the orientation of the collagen fibrils can be controlled to direct polymerization of the collagen fibrils in a desired manner.

For example, previous studies have shown that microfluidic channels of different diameters can direct the polymerization of collagen fibrils to form fibers that are oriented along the length of the channels, but only at channel diameters of 100 μm or less (Lee et al., 2006). Primary endothelial cells grown in these oriented matrices were shown to align in the direction of the collagen fibers. In another study, Martinez et al. demonstrate that 500 μm channels within a cellulose-bead scaffold can direct collagen and cell alignment (Martinez et al., 2012). By modulating the fiber diameter, the orientation of the collagen fibers within the fiber structure can be controlled. As such, the fiber structures, and the collagen fibers within them, can therefore be patterned to produce tissue constructs with a desired arrangement of collagen fibers, essential for conferring desired biomechanical properties on a 3D printed structure.

### Additional Fluids

Aspects of the invention include one or more buffer solutions. Buffer solutions in accordance with embodiments of the invention are miscible with an input material (e.g., a hydrogel) and do not cross-link the input material. In some embodiments, a buffer solution comprises an aqueous solvent. Non-limiting examples of buffer solutions include polyvinyl alcohol, water, glycerol, propylene glycol, sucrose, gelatin, or any combination thereof.

Buffer solutions in accordance with embodiments of the invention can have a viscosity that ranges from about 1 mPa·s to about 5,000 mPa·s, such as about 10, 20, 30, 40, 50, 60, 70, 80, 90, 100, 200, 300, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, or 4,750 mPa·s. In some embodiments, the viscosity of a buffer solution can be modulated so that it matches the viscosity of one or more input materials.

Aspects of the invention include one or more sheath fluids. Sheath fluids in accordance with embodiments of the invention are fluids that can be used, at least in part, to envelope or “sheath” an input material being dispensed from a dispensing channel. In some embodiments, a sheath fluid comprises an aqueous solvent. Non-limiting examples of sheath fluids include polyvinyl alcohol, water, glycerol, propylene glycol, sucrose, gelatin, or any combination thereof. Sheath fluids in accordance with embodiments of the invention can have a viscosity that ranges from about 1 mPa·s to about 5,000 mPa·s, such as about 10, 20, 30, 40, 50, 60, 70, 80, 90, 100, 200, 300, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, or 4,750 mPa·s. In some embodiments, the viscosity of a sheath fluid can be modulated so that it matches the viscosity of one or more input materials.

In some embodiments, a sheath fluid comprises a chemical cross-linking agent. In some embodiments, a chemical cross-linking agent comprises a divalent cation. Non-limiting examples of divalent cations include Cd²⁺, Ba²⁺, Cu²⁺, Ca²⁺, Ni²⁺, Co²⁺, or Mn²⁺. In a preferred embodiment, Ca²⁺ is used as the divalent cation. In some embodiments, the concentration of a divalent cation in the sheath fluid ranges from about 80 mM to about 140 mM, such as about 90, 100, 110, 120 or 130 mM.

### Cell Populations

In embodiments, the cell population is selected from the group comprising or consisting of a single-cell suspension, cell aggregates, cell spheroids, cell organoids, or combinations thereof. Input materials in accordance with embodiments of the invention can incorporate any mammalian cell type, including but not limited to stem cells (e.g., embryonic stem cells, adult stem cells, induced pluripotent stem cells), germ cells, endoderm cells (e.g., lung, liver, pancreas, gastrointestinal tract, or urogenital tract cells), mesoderm cells (e.g., kidney, bone, muscle, endothelial, or heart cells), ectoderm cells (skin, nervous system, pituitary, or eye cells), stem cell-derived cells, or any combination thereof.

For example, an input material can comprise cells from endocrine and exocrine glands including pancreas (alpha, beta, delta, epsilon, gamma), liver (hepatocyte, Kuppfer, Stelate, sinusoidal cells), thyroid (Follicular cells), pineal gland (pinealocytes), pituitary gland (somatotropes, Lactotropes, gonadotropes, corticotropes, and thyrotropes), thymus (thymocytes, thymic epithelial cells, thymic stromal cells), adrenal gland (cortical cells, chromaffin cells), ovary (granulosa cells), testis (Leydig cells), gastrointestinal tract (enteroendocrine cells intestinal, gastric, pancreatic), fibroblasts, chondrocytes, meniscus fibrochondrocytes, bone marrow stromal (stem) cells, embryonic stem cells, mesenchymal stem cells, induced pluripotent stem cells, differentiated stem cells, tissue-derived cells, smooth muscle cells, skeletal muscle cells, cardiac muscle cells, epithelial cells, endothelial cells, myoblasts, chondroblasts, osteoblasts, osteoclasts, and any combinations thereof.

Cells can be obtained from donors (allogenic), from a different species to the recipient (xenogeneic), or from recipients (autologous). Specifically, in embodiments, cells can be obtained from a suitable donor, such as a human or animal, or from the subject into which the cells are to be implanted. Mammalian species include, but are not limited to, humans, monkeys, dogs, cows, horses, pigs, sheep, goats, cats, mice, rabbits, and rats. In one embodiment, the cells are human cells. In other embodiments, the cells can be derived from animals such as dogs, cats, horses, monkeys, or any other mammal.

In some embodiments, the at least one biological material comprises a cell population expressing/secreting one or more endogenous biologically active agent(s), e.g., insulin, glucagon, ghrelin, pancreatic polypeptide, Factor VII, Factor VIII, Factor IX, alpha-1-antitrypsin, an angiogenic factor, a growth factor, a hormone, an antibody, an enzyme, a protein, an exosome, and the like. Discussed herein, endogenous biologically active agents comprise those agents that the cell naturally produces in a biological context (e.g., insulin release in response to elevated glucose concentrations). An endogenous biologically active agent can constitute a therapeutic agent in the context of the present disclosure.

In some embodiments, an input material can comprise genetically engineered cells that secrete specific factors. It is within the scope of this disclosure that a cell population as discussed above can comprise, in embodiments, engineered cells (e.g., genetically engineered cells) that secrete specific factors. Cells can also be from established cell culture lines, or can be cells that have undergone genetic engineering and/or manipulation to achieve a desired genotype or phenotype. In some embodiments, pieces of tissue can also be used, which may provide a number of different cell types within the same structure.

Genetic engineering techniques applicable to the present disclosure can include but are not limited to recombinant DNA (rDNA) technology (Stryjewska et al., Pharmacologial Reports. 2013; 65: 1075), cell-engineering based on use of targeted nucleases (e.g., meganuclease, zinc finger nucleases (ZFN), transcription activator-like effector nucleases (TALEN), clustered regularly interspaced short palindromic repeat-associated nuclease Caso (CRISPR-Cas₉), etc. (Lim et al., Nature Communications. 2020; 11: 4043; Stoddard B L, Structure. 2011; 19(1): 7-15; Gaj et al., Trends Biotechnol. 2013; 31(7): 397-405; Hsu et al., Cell. 2014; 157(6): 1262; Miller et al., Nat Biotechnol. 2010; 29(2): 143-148), cell-engineering based on use of site-specific recombination using recombinase systems (e.g., Cre-Lox) (Osborn et al., Mol Ther. 2013; 21(6): 1151-1159; Hockemeyer et al., Nat Biotechnol. 2009; 27(9): 851-857; Uhde-Stone et al., RNA. 2014; 20(6): 948-955; Ho et al., Nucleic Acids Res. 2015; 43(3): e17; Sengupta et al., Journal of Biological Engineering. 2017; 11(45): 1-9), and the like. In some embodiments, some combination of the above-mentioned techniques for cell-engineering may be used.

Encompassed by the present disclosure are engineered cells capable of producing one or more therapeutic agents, including but not limited to proteins, peptides, nucleic acids (e.g., DNA, RNA, mRNA, siRNA, miRNA, nucleic acid analogs), peptide nucleic acids, aptamers, antibodies or fragments or portions thereof, antigens or epitopes, hormones, hormone antagonists, growth factors or recombinant growth factors and fragments and variants thereof, cytokines, enzymes, antibiotics or antimicrobial compounds, anti-inflammation agent, antifungals, antivirals, toxins, prodrugs, small molecules, drugs (e.g., drugs, dyes, amino acids, vitamins, antioxidants) or any combination thereof.

In embodiments, cells of the present disclosure may be modified to comprise at least one mechanism for providing a local immunosuppression at a transplant site when transplanted in an allogeneic host, for example in tissue fibers of the present disclosure. In examples, a cell or cells may comprise a set of transgenes, each transgene encoding a gene product that is cytoplasmic, membrane bound, or local acting, and whose function can include but is not limited to mitigate antigen presenting cell activation and function; to mitigate graft attacking leukocyte activity or cytolytic function; to mitigate macrophage cytolytic function and phagocytosis of allograft cells; to induce apoptosis in graft attacking leukocytes; to mitigate local inflammatory proteins; and to protect against leukocyte-mediated apoptosis (WO2018/227286; Harding et al., BioRxiv. 2019; DOI: 10.1101/716571; Lanza et al., Nature Reviews Immunology. 2019; 19: 723-7331; Harding et al., Cell Stem Cell. 2020; 27(2): 198-199).

In embodiments, cells of the present disclosure may be modified in a manner to exert control over cell proliferation. As an example, a cell may be genetically modified at a cell division locus (CDL) to comprise a negative selectable marker and/or an inducible activator-based gene expression system, thereby enabling control over the permitting, ablation and/or inhibition of proliferation of the genetically modified cells by addition or removal of an appropriate inducer (WO2016/141480; Liang et al., Nature. 2018; 563(7733): 701-704).

Appropriate growth conditions for mammalian cells are well known in the art (Freshney, R. I. (2000) Culture of Animal Cells, a Manual of Basic Technique. Hoboken N.J., John Wiley & Sons; Lanza et al. Principles of Tissue Engineering, Academic Press; 2nd edition May 15, 2000; and Lanza & Atala, Methods of Tissue Engineering Academic Press; 1st edition October 2001). Cell culture media generally include essential nutrients and, optionally, additional elements such as growth factors, salts, minerals, vitamins, etc., that may be selected according to the cell type(s) being cultured. Particular ingredients may be selected to enhance cell growth, differentiation, secretion of specific proteins, etc. In general, standard growth media include Dulbecco's Modified Eagle Medium, low glucose (DMEM), with 110 mg/L pyruvate and glutamine, supplemented with 10-20% fetal bovine serum (FBS) or calf serum and 100 U/ml penicillin are appropriate as are various other standard media well known to those in the art. Growth conditions will vary depending on the type of mammalian cells in use and the tissue desired.

In some embodiments, cell-type specific reagents can be advantageously employed in the subject input materials for use with a corresponding cell type. For example, an extracellular matrix (“ECM”) can be extracted directly from a tissue of interest and then solubilized and incorporated it into an input material to generate tissue-specific input materials for printed tissues. Such ECMs can be readily obtained from patient samples and/or are available commercially from suppliers such as zPredicta (rBone™, available at zpredicta.com/home/products).

### Printing Systems

Although bioprinting systems vary, they generally include at least one reservoir comprising an input material (e.g., bioink, sheath fluid, buffer, etc.) for dispensing by way of a dispensing orifice (e.g., dispensing orifice associated with a needle, nested needle, syringe, nozzle, and the like). Dispensing orifice(s) can comprise suitable shape(s), for example circular, square, oval, oblong, rectangular, and the like. In some examples, a bioprinting system comprises a plurality of reservoirs and/or can include means for selecting a reservoir to be employed in bioprinting from a plurality of reservoirs. Bioprinting systems that include one or more reservoirs can be advantageous in continuous or substantially continuous bioprinting applications. Volumes of reservoirs vary, for example between about 100 pl and about 1 L or more, including any intervening value therein, such as about 1, 5, 10, 15, 20, 25, 30, 35, 40, 45, 50, 55, 60, 65, 70, 75, 80, 85, 90, 95 or 100 mL, or such as about 150, 200, 250, 300, 350, 400, 450, 500, 550, 600, 650, 700, 750, 800, 850, 900 or 950 mL.

In examples, reservoirs can be comprised of a material on the inside that resists attachment of cells thereto. In examples, at least the inside of reservoirs can be comprised of biocompatible material. Reservoirs may be compatible with bioprinting that involves extruding a semi-solid or solid bio-ink or a support material through one or more dispensing orifices. Reservoirs may be compatible with bioprinting that involves dispensing a liquid or semi-solid cell solution, cell suspension, or cell concentration through one or more dispensing orifices. Reservoirs may be compatible with non-continuous bioprinting, continuous and/or substantially continuous bioprinting. Reservoirs can include but are not limited to a capillary tube, micropipette, syringe, needle, bottle, basin, receptacle, and the like. Many internal diameters are suitable for substantially round or cylindrical reservoirs.

Reservoirs of bioprinting systems may in some examples be primed. For example, priming a reservoir can increase accuracy of the dispensing and/or deposition process by e.g., compacting and advancing the contents of the reservoir until the material to be dispensed (e.g., bioink) is located in a position in contact with the dispensing orifice.

In preferred embodiments, bioprinting systems comprise technology as described in WO2014/197999, WO2018/165761, WO2020/056517, WO2021/081672, and U.S. Provisional Patent Application No. 63/290,595, the disclosures of which are expressly incorporated herein by reference. As detailed therein, the disclosed bioprinting systems and components thereof enable multi-material switching, and hence the composition of one or more components (e.g., cell type, biomaterial composition) of the synthetically generated tissue fiber can be modified along the length of the fiber while continuously printing. In embodiments, a microfluidics-based bioprinting system is the RX1™ bioprinter (Aspect Biosystems, Vancouver, BC, Canada).

In an exemplary embodiment of a preferred bioprinting system, the system comprises a print head comprising a dispensing channel, wherein one or more material channels and a core channel converge at the proximal end of the dispensing channel. A print head may be configured to dispense buffer solution and/or sheath fluid simultaneous with one or more cross-linkable materials. In some embodiments, a print head is configured to maintain a constant mass flow rate through the dispensing channel. In this manner, a print head can be configured to facilitate a smooth and continuous flow of one or more input materials (or a mixture of one or more input materials) and a buffer solution and/or sheath fluid through the dispensing channel. In use of such a print head, an input material flowing through the dispensing channel can be cross-linked from the inside, by a fluid flowing through the core channel and/or from the outside, by sheath fluid flowing through a downstream sheath fluid channel, as described more particularly in WO2020/056517. In some embodiments, a print head comprises one or more fluidic focusing chambers comprised of a conical frustum shape and, optionally, one or more print head adaptors, as described in detail in WO 2021/081672, and U.S. Provisional Patent Application No. 63/290,595. In embodiments, a print head is the DUO™ microfluidic printhead, or the CENTRA™ microfluidic printhead (Aspect Biosystems, Vancouver, BC, Canada).

Other examples of bioprinting systems relevant in the context of the present disclosure include but are not limited to 3-D Bioplotter® (EnvisionTEC Inc., Dearborn, MI, USA), NovoGen Bioprinter® Platform (Organovo®, San Diego, CA, USA), R-Gen 100 and R-Gen 200 (RegenHU, Villas-Saint-Pierre, Switzerland), Bioprinter Fabion and Fabion 2 (3D Bioprinting Solutions, Moscow, Russia), BioBot® Basic, BioAssemblyBot® 200/400/500 (Advanced Solutions, Louisville, KY, USA), BIO X™, BIO X6™, INKREDIBLE+™ (CellINK, Boston, MA, USA), Ourobotics Revolution (Ourobotics, Cork, Ireland), BioScaffolder 2.1(GeSim, Radeberg, Germany), Omega Bioprinter (3Dynamic Systems, Bridgend, UK), Syn\hat{ } and Explorer (Bio3D, Singapore), Alevi 1/2/3 (Alevi by 3D Systems, Rock Hill, SC, USA), and Dr. Invivo 4D6 (Rokit Healthcare, Seoul, South Korea).

As discussed above, bioprinting systems typically dispense bioprinted material onto a receiving surface. Discussed herein, bioprinted fiber structures are printed using a means for suspending the bioprinted fiber structure during one or more of printing, patterning and/or processing. In embodiments, a fiber structure can be printed onto a receiving surface (e.g., mesh 514 at FIGS. 5A-5B) via the use of a means for suspending said structure (e.g., frame 508 at FIGS. 5A-5B). In embodiments, a fiber structure can be printed by way of a means for suspending said structure as herein disclosed, such that the fiber structure remains completely suspended off of a receiving surface during printing.

Bioprinting systems as herein disclosed can be modified or otherwise adapted to provide surfaces capable of accommodating the herein disclosed means for suspending a bioprinted tissue structure. In embodiments, a surface may be adapted or otherwise configured in a manner so as to operably couple to one or more vessels (e.g., container(s), multi-well plate, and the like). For example, and without limitation, a container may include a container capable of holding a solution such as a cross-linker solution, or a dip-coat solution as herein described. In embodiments, such a container may be of a size and shape to enable an entirety of the means for suspending a bioprinted fiber to fit therein, and where a liquid held in the container is capable to completely submerge the means for suspending a bioprinted fiber, and in turn, completely submerge the bioprinted fiber structure attached thereto.

In embodiments, a receiving surface is disposable. In embodiments, a receiving surface can be effectively sterilized. In embodiments, a receiving surface comprises a solid material, a semi-solid material, or some combination thereof. In embodiments, the receiving surface is porous. In embodiments, the receiving surface comprises glass, coated glass, plastic, coated plastic, metal, a metal alloy, mesh, grate, or a combination thereof.

In some embodiments, a bioprinting system comprises a fluid removal component for removing excess fluid (e.g., excess sheath fluid and/or excess buffer solution) from a receiving surface and/or from a surface of a dispensed tissue fiber structure. During printing, it is possible that excess fluid may collect or “pool” on a receiving surface or on a surface of dispensed tissue fiber structure, and in some examples such pooling may interfere with one or more aspects of the deposition process. For example, in the context of the present disclosure, undesired excess fluid can potentially degrade the ability of a fiber structure to adhere to posts (e.g., posts 206 at FIG. 2A) of a means for suspending a bioprinted fiber structure (e.g., frame 202 at FIG. 2), may add undesired weight (possibly unevenly distributed) to a bioprinted fiber, and the like, which may cause a dispensed fiber to slip from its intended position in a 3D structure being printed. Therefore, in some embodiments, removal of excess sheath fluid from the receiving surface and/or from a surface of the dispensed fiber structure by way of a fluidic removal component may improve additive manufacturing of three-dimensional structures.

Excess fluid may be removed from a receiving surface or from a surface of one or more layers of dispensed fibers by drawing the fluid off of those surfaces, by allowing or facilitating evaporation of the fluid from those surfaces or, in embodiments where the receiving surface is porous, excess fluid may be removed by drawing it through the porous surface. In some embodiments, an absorptive material (e.g., a sponge) can be used to draw excess fluid away from a receiving surface.

In some embodiments, a receiving surface comprises a vacuum component (e.g., vacuum chuck 512 at FIGS. 5A-5B) that is configured to apply suction from one or more vacuum sources to the receiving surface. In some embodiments, a receiving surface comprises one or more vacuum channels that are configured to apply suction to the receiving surface. In some embodiments, a receiving surface comprising a vacuum component is configured to aspirate an excess fluid from the receiving surface before, during and/or after a printing process is carried out. In some embodiments where the receiving surface is porous, the vacuum component may be configured to apply suction to draw excess fluid through the porous surface.

In some embodiments, a receiving surface comprises one or more tubes that are fluidly coupled to a vacuum source, which can provide suction for removing excess fluid from the receiving surface, and optionally from a surface of dispensed fiber structure. In such embodiments, a solid or porous receiving surface can also be used. In some embodiments, a print head (e.g., a microfluidics-based print head) is configured to further comprise one or more vacuum channels, the one or more vacuum channels each having an orifice situated near (i.e., adjacent to) the dispensing orifice. When the print head is in fluid communication with a vacuum, the one or more vacuum channels may direct negative pressure to an area of a receiving surface where materials are being dispensed or have been dispensed from the dispensing orifice and/or to a portion of the surface area of a dispensed fiber structure, thereby drawing up excess fluid from the receiving surface and/or from a surface of the dispensed fiber structure.

In some embodiments, the bioprinter system may employ a dispensing means to control material flow in fiber production. For example, the bioprinter system may employ material displacement to control material flow. In embodiments, said dispensing means provides a force to dispense one or more materials to be dispensed. In one embodiment, the dispensing means provides pneumatic pressure to supply the force to dispense one or more materials. In some embodiments, the bioprinter system may be configured with one or more pumps. Examples include but are not limited to gear pumps, peristaltic pumps, lobe pumps, piston pumps (e.g., dual piston pump), syringe pumps, and the like. In some embodiments, a pump is used to exert a force on material contained in a reservoir to assist in moving the material toward an associated dispensing orifice.

In a preferred example, a bioprinter system used in the context of the present disclosure comprises a radial pump assembly configured to minimize internal volume in a multi-channel bioprinting system, preferably a microfluidic bioprinting system, comprising a plurality of pumps positioned in a radial array on a mounting bracket, wherein each pump of the plurality of pumps comprises a housing and a retainer for securing the pump. A relevant example of such a system is described in detail in U.S. Provisional Patent Application No. 63/290,595, the contents of which is herein incorporated by reference in its entirety.

In embodiments, bioprinting systems relevant to the present disclosure can comprise some type of enclosure system. The enclosure system may be configured to enclose a portion or an entirety of a bioprinting system. Preferably, the enclosure system encloses at least an area in which a bioprinted fiber is dispensed, for example at least a receiving surface, preferably at least a receiving surface and one or more print heads, optionally enclosing any dispensing means (e.g., syringe pump(s)) configured to deliver a flow material to a dispensing orifice, optionally enclosing one or more reservoir(s). In embodiments, such an enclosure system can be configured to adopt an open configuration where aspects of the bioprinting system can be accessed, and a closed configuration where particular aspects of the bioprinting system are sealed off, or substantially sealed off, from surrounding atmosphere.

Reliance on an enclosure system may enable accurate control over parameters including but not limited to temperature, humidity, O₂, and CO₂. Accurate control over such parameters can enable increased stability and integrity of 3D bioprinted structures, as well as increased cellular survival during and after a bioprinting process under conditions where the 3D bioprinted structure includes cellular material. In embodiments, variables such as temperature, humidity, O₂, and CO₂ can be controlled within the enclosure system by way of a feedback control system (e.g., proportional integral derivative (PID) controller), see, e.g., Matamoros M, et al. (2020) Micromachines, 11, 999. In embodiments, such a feedback control system can, e.g., stabilize temperature within the enclosure system to a desired temperature (within some margin of error) and/or stabilize humidity within the enclosure system to a desired humidity (within some margin of error). In some additional or alternative embodiments, such a feedback control system can stabilize CO₂ to a desired level (e.g., desired ppm within some margin of error). In some additional or alternative embodiments, such a feedback control system can stabilize O₂ to a desired level (e.g., desired ppm within some margin of error).

Thus, in embodiments, a bioprinting system relevant to the present disclosure may comprise one or more of a temperature modulation component, humidity modulation component, an O₂ modulation component, and a CO₂ modulation component. In embodiments, the temperature modulation component comprises a heater (e.g., radiant heater, convection heater, conductive heater, fan heater, heat exchanger, or any combination thereof). In embodiments, the temperature modulation component comprises a cooling element (e.g., coolant, chilled liquid, Peltier cooler, radiant cooler, a convection cooler, a conductive cooler, a fan cooler, or any combination thereof). The humidity modulation component can comprise, for example, a chamber (e.g., tank) of water that can be vaporized via a piezoelectrical transducer. Control of O₂ may be by way of O₂ injection. Control of CO₂ may be by way of CO₂ injection. In embodiments, the temperature modulation component is capable to adjust and/or maintain the temperature within the enclosure system and/or one or more of a print head, a printer stage, a receiving surface, a flow material, and/or a fluid (e.g., a sheath solution and/or a buffer solution). In embodiments, the temperature modulation component is configured to adjust a temperature to a set point that ranges from about 0 to about 90° C., such as about 1, 2, 3, 4, 5, 10, 15, 20, 25, 30, 35, 40, 45, 50, 55, 60, 65, 70, 75, 80 or 85° C. In embodiments, the humidity modulation component is configured to adjust humidity to between about 30% and 100%, such as about 35%, 40%, 45%, 50%, 55%, 60%, 65%, 70%, 75%, 80%, 85%, 90%, 95%. In embodiments, the CO₂ modulation component is configured to adjust CO₂ levels to between about 2% to about 15%, such as about 3%, 4%, 5%, 6%, 7%, 8%, 9%, 10%, 11%, 12%, 13%, 14%.

In embodiments, a bioprinting system relevant to the present disclosure achieves a particular geometry of a dispensed fiber structure by moving a printer stage or surface (e.g., receiving surface) atop which sits a means for suspending a bioprinted fiber structure, relative to a dispensing orifice. In alternative embodiments, a bioprinting system relevant to the present disclosure achieves a particular geometry of a dispensed fiber structure by moving a dispensing orifice (optionally a plurality thereof) relative to a printer stage or surface (e.g., receiving surface) atop or above which sits a means for suspending a bioprinted fiber structure. In certain embodiments, at least a portion of a bioprinting system is maintained in a sterile environment (e.g., within a biosafety cabinet (BSC)). In some embodiments, a bioprinting system is configured to fit entirely within a sterile environment.

In some embodiments, a bioprinting system comprises a 3D motorized stage, also referred to herein as a positioning unit, comprising at least three arms for positioning a surface (e.g., receiving surface) atop which sits a means for suspending a bioprinted fiber structure in three dimensional space (i.e., along x, y, and z axes of a Cartesian coordinate system) below a dispensing orifice. In some additional or alternative embodiments, a similar positioning unit positions a dispensing orifice in three dimensional space above a surface (e.g., receiving surface) atop or above which sits a means for suspending a bioprinted fiber structure.

In some embodiments, the 3D motorized stage arms are driven by corresponding motors, respectively, and controlled by a programmable control processor, such as a computer. In an embodiment, a surface (e.g., receiving surface) atop with sits a means for suspending a bioprinted fiber structure is moveable along all three primary axes of the Cartesian coordinate system by a 3D motorized stage, and movement of the stage is defined using computer software. In some additional or alternative embodiments, a dispensing orifice (or plurality thereof) is moveable along all three primary axes of the Cartesian coordinate system by a 3D motorized stage, and movement of the stage is defined using computer software.

It is to be understood that the invention is not limited to only the described positioning system, and that other positioning systems are known in the art. As material is dispensed from a dispensing orifice (e.g., on a microfluidic print head), the positioning unit is moved in a pattern controlled by software, thereby creating a first layer of a bioprinted fiber dispensed around posts (e.g., posts 206 at FIG. 2A) of a means for suspending a bioprinted fiber (e.g., frame 202 at FIG. 2A). Additional layers of dispensed material are then stacked on top of one another such that the final 3D geometry of the dispensed layers of material is generally a replica of a 3D geometry design provided by the software. The 3D design may be created using typical 3D CAD (computer aided design) software or generated from digital images, as known in the art. Further, if the software generated geometry contains information on specific materials to be used, it is possible, according to one embodiment of the invention, to assign a specific flow material type to different geometrical locations. For example, in some embodiments, a printed 3D structure can comprise two or more different input materials, wherein each input material has different properties (e.g., each input material comprises a different cell type, a different cell concentration, a different extracellular matrix (ECM) composition, a different type of material (e.g., different type of hydrogel material), etc.).

In some embodiments, a bioprinting system comprises a light module for optionally exposing a photo cross-linkable flow material to light in order to cross-link the material. Light modules (e.g., ultraviolet (UV) light module) can in embodiments be integrated into a bioprinting system (e.g., into a print head thereof), or can be a standalone component of such a system. In some embodiments, a light module is ring-shaped. In some embodiments, a ring-shaped light module entirely surrounds a transparent portion of a dispensing channel (or dispensing needle/nozzle/syringe) and/or dispensing orifice. In some embodiments, the ring-shaped light module is positioned directly below a dispensing orifice.

In embodiments, a ring-shaped module can be configured with a plurality of light sources to direct light inward in the direction of a central cavity of the ring-shaped light module, such that light is directed circumferentially at a fiber as the fiber is being printed. In embodiments, the plurality of light sources comprises at least between 10 and 40 individual light sources, for example between 15 and 35, for example between 20 and 30. In embodiments, the light sources comprise light-emitting diodes (LEDs), for example UV-LEDs. A relevant example of such a ring-shaped light source and its incorporation into a bioprinting system is described in detail in U.S. Provisional Patent Application No. 63/290,595, the contents of which is herein incorporated by reference in its entirety.

Aspects of a bioprinting system include software programs that are configured to facilitate deposition of the subject flow materials in a specific pattern and at specific positions in order to form a specific planar or 3D structure in a manner whereby the structure is deposited around posts (e.g., posts 206 at FIG. 2A) of a means for suspending a bioprinted fiber (e.g., frame 202 at FIG. 2A) as herein described. In order to fabricate such structures, the subject printing systems deposit the subject input materials in a precise manner (in two or three dimensions) by weaving a continuous bioprinted fiber in a defined pattern around at least two, optionally more than two, optionally more than 10 posts, optionally more than 15, optionally more than 20, optionally more than 25, optionally more than 30, optionally more than 35, optionally more than 40, optionally more than 45, optionally more than 50, optionally more than 75, optionally more than 100 posts (e.g., posts 206 at FIG. 2) that are part of a means for suspending a bioprinted fiber structure (e.g., frame 202 at FIG. 2) and/or fabrication platform (e.g., fabrication platform 500 at FIGS. 5A-5B). The resultant structure may comprise one, two, three, four, five, or more than five layers, for example 10 layers or more, 20 layers or more, 30 layers or more, 40 layers or more, 50 layers or more, 60 layers or more, 70 layers or more, 80 layers or more, 90 layers or more, 100 layers or more, 110 layers or more, 120 layers or more, 130 layers or more, 140 layers or more, 150 layers or more, 160 layers or more, 170 layers or more 180 layers or more, 190 layers or more, 200 layers or more. In some embodiments, the manner (i.e., locations and overall deposition pattern) in which a printing system deposits a material are defined by a user input, and are translated into computer code. In some embodiments, a computer code includes a sequence of instructions, executable in the central processing unit (CPU) of a digital processing device, written to perform a specified task. In some embodiments, printing parameters including, but not limited to, printed fiber dimensions, pump speed, movement speed of the positioning unit, and crosslinking agent intensity or concentration are defined by user inputs and are translated into computer code. In some embodiments, printing parameters are not directly defined by user input, but are derived from other parameters and conditions by the computer code.

Aspects of the present invention include methods for fabricating fiber structures, comprising: a computer module receiving input of a visual representation of a desired tissue construct; a computer module generating a series of commands, wherein the commands are based on the visual representation and are readable by a bioprinting system as herein disclosed; a computer module providing the series of commands to a bioprinting system; and the printing system depositing one or more input materials according to the commands to form a fiber structure with a defined geometry.

In some embodiments, the manner (i.e., locations and overall deposition pattern) at which a bioprinting system deposits an input material are defined by a user input and are translated into computer code. In some embodiments, the devices, systems, and methods disclosed herein further comprise non-transitory computer readable storage media or storage media encoded with computer readable program code. In some embodiments, a computer readable storage medium is a tangible component of a bioprinting system (or a component thereof) or a computer connected to a bioprinting system (or a component thereof). In some embodiments, a computer readable storage medium is optionally removable from a digital processing device. In some embodiments, a computer readable storage medium includes, by way of non-limiting example, a CD-ROM, DVD, flash memory device, solid state memory, magnetic disk drive, magnetic tape drive, optical disk drive, cloud computing system and/or service, and the like. In some cases, the program and instructions are permanently, substantially permanently, semi-permanently, or non-transitorily encoded on a storage medium.

In some embodiments, the devices, systems, and methods described herein comprise software, server, and database modules. In some embodiments, a “computer module” is a software component (including a section of code) that interacts with a larger computing system. In some embodiments, a software module (or program module) comes in the form of one or more files and typically handles a specific task within a computing system.

In some embodiments, a module is included in one or more software systems. In some embodiments, a module is integrated with one or more other modules into one or more software systems. A computer module is optionally a stand-alone section of code or, optionally, code that is not separately identifiable. In some embodiments, the modules are in a single application. In other embodiments, the modules are in a plurality of applications. In some embodiments, the modules are hosted on one machine. In some embodiments, the modules are hosted on a plurality of machines. In some embodiments, the modules are hosted on a plurality of machines in one location. In some embodiments, the modules are hosted a plurality of machines in more than one location. Computer modules in accordance with embodiments of the invention allow an end user to use a computer to perform the one or more aspects of the methods described herein.

In some embodiments, a computer module comprises a graphical user interface (GUI). As used herein, “graphic user interface” means a user environment that uses pictorial as well as textual representations of the input and output of applications and the hierarchical or other data structure in which information is stored. In some embodiments, a computer module comprises a display screen. In further embodiments, a computer module presents, via a display screen, a two-dimensional GUI. In some embodiments, a computer module presents, via a display screen, a three-dimensional GUI such as a virtual reality environment. In some embodiments, the display screen is a touchscreen and presents an interactive GUI.

### Quality Control Systems

Quality assurance for 3D bioprinting of fibers via printing systems, such as those disclosed herein, is vital to reproducible biofiber fabrication, function, and regulatory approval for any translational application. Accordingly, bioprinting systems of the present disclosure can incorporate one or more of the below-discussed quality control systems. In embodiments, a quality control system comprises one or more cameras. In embodiments where at least a portion of a dispensing orifice (and/or dispensing channel/needle/syringe/nozzle leading thereto) and/or other aspect of the system (e.g., microfluidic print head) is transparent, one or more cameras can be used to image the flow of material to be dispensed, for purposes of detecting a clog or other aberration in material flow.

For example, in embodiments a microfluidic print head of the present disclosure (e.g., similar to or substantially the same as DUO™ microfluidic printhead or CENTRA™ microfluidic printhead (Aspect Biosystems, Vancouver, BC, Canada)) comprises a transparent dispensing channel. In such embodiments, a camera system may comprise a first camera positioned at a first angle with respect to the transparent dispensing channel, and a second camera positioned at a second, different angle with respect to the transparent dispensing channel. In embodiments, the two cameras may be oriented at about a 90° angle with respect to one another. The first camera and the second camera can form part of a machine-learning based system capable to identify one or more deviations in the material flow from user-established material flow parameters. For example, such a system may be capable to enable monitoring of fiber concentricity, various fiber properties, presence or absence of clogs, bubbles, and the like, and may further be capable of controlling one or more parameters (e.g., valve opening/closing, material flow rate, and the like) based on said monitoring. This concept and variations thereof are described in U.S. Provisional Application No. 63/238,028, the contents of which are incorporated by reference herein in their entirety.

In embodiments, one or more additional or alternative cameras can be included as part of a bioprinting system of the present disclosure. In one such embodiment, a camera(s) can be aimed at the means for suspending a bioprinted fiber structure (e.g., frame 202 at FIG. 2) so as to be capable to image the bioprinted fiber structure as it is printed, and monitor one or more properties associated with said fiber during printing. Such properties can include but are not limited to presence and/or absence of leading and/or trailing fibers and shape fidelity of printed fibers and/or 3D structures formed from said printed fibers.

In embodiments, a ring-shaped light module such as that discussed above may comprise a part of a quality control system, in that the ring-shaped light module may serve to ensure uniformity of cross-linking of a fiber comprised of a photo cross-linkable material as the fiber is printed.

It is also herein recognized that use of pneumatic valves can be advantageous in the context of the present disclosure in terms of reducing or avoiding inaccuracies or distortions of desired 3D structures. For example, reliance on one or more pneumatic valves in conjunction with printing systems of the present disclosure can reduce or avoid inaccurate commencement and/or cessation of the printing process, which may otherwise result in the formation of leading and/or trailing fibers that may distort the dimensions of printed fibers and 3D structures. Reliance on pneumatic valves can also be advantageous in fibers that are comprised of more than one type of material along a length of the fiber, for accurate switching of the type of material from one to another without the formation of leading and/or trailing ends. Such accurate switching can enable effective compartmentalization of different segments along the length of a fiber. For example, a fiber may comprise a first segment comprised of a first type of hydrogel, a second segment comprised of a second type of hydrogel, where the second segment optionally includes cells, and a third segment comprised of yet another type of hydrogel. Such an example is meant to be illustrative, and the skilled artisan can select the types of materials and, optionally, cell type(s) (or lack thereof) for various compartments along the length of a fiber as desired for a particular application.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

### Methods of Making

Aspects of the invention include methods of printing a linear fiber structure, a planar structure comprising one or more fiber structures, or a three-dimensional (3D) structure comprising two or more layers of planar structures. The manner of printing a first layer and, optionally a second layer, third layer, and so on (e.g., 200 layers or more is within the scope of this disclosure), is described in detail above with reference to FIGS. 3-4 and 6. In some embodiments, a linear fiber structure may be created by wrapping a bioprinted fiber one or more times around a first post (e.g. post 206 of FIG. 2A), then continuing to print the fiber and wrapping the fiber one or more times around a second post, preferably wherein the first post and second post are on opposing sides of a means for suspending a bioprinted fiber structure (e.g., frame 202 of FIG. 2A). In some embodiments, a method first comprises providing a design for a linear, planar, or 3D structure to be printed. The design can be created using commercially available CAD software. In some embodiments, the design comprises information regarding specific materials (e.g., for heterogeneous structures comprising multiple materials) to be assigned to specific locations in the structure(s) to be printed.

In embodiments, a method comprises dispensing a bioprinted fiber into a cross-linker bath, where the fiber is printed via a means for suspending a bioprinted fiber (e.g., frame 202 at FIG. 2), and wherein the means, and in turn, the resultant fiber is submerged in the cross-linker bath. In some embodiments of such a method, the bioprinted fiber may be additionally cross-linked prior to being dispensed into the cross-linker bath. For example, the bioprinted fiber may be cross-linked via sheath fluid containing cross-linker, or via photo cross-linking, prior to being introduced into the cross-linker bath. In other embodiments, the bioprinted fiber may not be cross-linked prior to being dispensed into the cross-linker bath. In still other embodiments, a bioprinted fiber may be produced in a manner where the fiber is cross-linked (e.g., exposed to sheath fluid comprising cross-linker and/or photo cross-linked) prior to being dispensed onto a means for suspending a bioprinted fiber, under conditions where the means is not positioned in a cross-linker bath. In some embodiments, following printing of the cross-linked fiber, the means for suspending and the correspondingly attached bioprinted fiber may then be submerged in a cross-linker bath. Such an embodiment is discussed above with regard to the exemplary process flow of FIG. 8.

In embodiments, a method comprises uniformly coating a bioprinted fiber structure to produce a conformal coating of the entirety of the external surface of the structure. In embodiments, the method comprises coating a a bioprinted fiber (e.g., 3D structure) that has been printed using a means for suspending (e.g., frame 202 at FIG. 2A) as herein disclosed, and applying a desired coating material while the fiber structure is suspended above the receiving surface by way of the means for suspending. In this way, a conformal coating comprising the coating material can be added to a suspended bioprinted fiber. In embodiments, a method comprises producing a cross linked bioprinted fiber structure (e.g., 3D structure) in any manner as described above, and then submerging an entirety of the fiber structure in a coating solution while the fiber structure remains attached to the means for suspending the bioprinted fiber structure.

In other additional or alternative embodiments, the coating solution can be applied (e.g., by way of a print head, such as a microfluidic print head) to a fiber structure while said structure remains attached to the means for suspending the fiber structure, and where the fiber structure is suspended off of a receiving surface. As discussed herein, in embodiments, a plurality (e.g., at least 2, 3, 4, 5, 6 or more) of coating layers can be applied to a bioprinted fiber.

In some embodiments of the methods described herein, a solution (e.g., a cross liker solution, buffer solution, coating solution) in which a bioprinted fiber is submerged therein can be removed via aspiration, or via draining (e.g., by way of one or more pluggable orifices). In this way, a single vessel may be used for submerging the bioprinted fiber into different solutions. For example, a bioprinted fiber attached to a means for suspending (e.g., frame 202 at FIG. 2A) may be submerged in a cross linker solution held in a vessel, and then the cross linker solution may be aspirated or otherwise removed, and then the vessel may be filled with another solution (e.g., a wash buffer, a coating solution, a storage solution, etc.). In this way, the means for suspending the bioprinted fiber, and the attached fiber itself, need not be moved from one vessel to another during various processing steps. A rate at which solutions are added and/or removed can be controlled so as to impart minimal (e.g., none, or substantially none) disturbance to the bioprinted fiber during the adding and/or removing.

In some embodiments of the methods described herein, to facilitate removal of one solution (e.g., cross linker solution) that a bioprinted fiber structure is submerged within, and then optionally, subsequent submerging of said fiber structure into another solution (e.g., coating solution) or otherwise processing (e.g., depositing a coating solution onto said fiber structure), an entirety of both the means for suspending (e.g., frame 202 at FIG. 2A) and the attached bioprinted fiber can be removed (i.e., lifted) from the first solution and, optionally then placed into a second solution or otherwise processed as discussed. In embodiments, such a process may be conducted manually. In embodiments, such a process may be conducted automatically (e.g., a robotically controlled process).

In embodiments, a fabrication platform such as that discussed above with regard to FIG. 7A can be used to alternately submerge and remove a fiber structure attached to a means for suspending said fiber structure, by manipulation of a lift arm (e.g., lift arm 702 at FIG. 7A). In other embodiments, a user can simply rely on, for example, a handle (e.g., handle 218 at FIG. 2B) coupled to a means for suspending a bioprinted fiber structure, to manually move a fiber structure attached to a means for suspending a bioprinted fiber structure from one environment to another. For example, a user can position said means and attached fiber structure in a bath, and subsequently remove said means and attached fiber structure. Such action can be repeated any number of times.

In embodiments, a method for bioprinting a fiber structure comprises printing a continuous length of at least one hydrogel fiber around two or more posts, for example 3, 4, 5, 6, 7, 8, 9, 10 or more, for example 20 or more, 30 or more, 40 or more, 50 or more, or even 100 or more (e.g., posts 206 at FIG. 2A) associated with a means for suspending a bioprinted fiber structure (e.g., frame 202 at FIG. 2A). In embodiments, the means is included as part of a fabrication platform as herein disclosed and/or included as part of a bioprinting system as herein disclosed. In embodiments, the method is used to generate a planar structure (i.e., one layer). In embodiments, the method is used to generate a 3D structure (i.e., two or more layers). In embodiments, the method further comprises conformal coating of an entirety of the bioprinted fiber structure, such that its upper and lower sides, as well as side walls, are uniformly coated. In embodiments, the method further comprises conformal coating of an entirety of the bioprinted fiber structure a plurality of times (e.g., at least 2). Each conformal coating layer can comprise a same or different coating material compositions.

In embodiments, the method for bioprinting the fiber structure comprises printing a fiber structure comprised of a core, and at least one shell layer surrounding said core. In embodiments, a coating material is applied to the entirety of the fiber structure to create a uniform conformal coating of the structure. The conformal coating may impart stability to the fiber structure, and/or may endow the structure with properties that optimize the interface between the fiber structure and host, e.g. anti-FBR properties, promotion of vascularization, etc. In embodiments, the coating may be softer than the outermost external shell. In embodiments, the core, external shell, and conformal coating may comprise from about 0.1% to about 4% alginate. In embodiments, the fiber structure may comprise a core of about 0.75%-1.5% alginate, an external shell of about 1.5-2.5% alginate, and a conformal coating of about 0.2-2.5%, such as 0.2-0.75%, alginate.

In embodiments wherein two coatings are applied, it is within the scope of this disclosure that the innermost coating may be of a greater hardness than outermost coating. In embodiments, the core, external shell, and conformal coatings may comprise from about 0.1% to about 4% alginate. In embodiments, the fiber structure may comprise a core of about 0.75%-1.5% alginate, an external shell of about 1.5-2.5% alginate, an inner conformal coating of about 1.5-2.5% alginate, and an outer conformal coating of about 0.2-2.5%, such as 0.2-0.75%, alginate. Such examples are meant to be illustrative.

In embodiments, a bioprinted fiber structure made by the methods herein disclosed can be segmented/compartmentalized along at least a portion of a length of a fiber, preferably a continuous fiber, that makes up the bioprinted fiber structure. Details of the production of segmented/compartmentalized bioprinted fiber structures is described in U.S. Provisional Patent Application No. 63/192,552, the contents of which is expressly incorporated by reference herein in its entirety. In embodiments, a fiber is comprised of a core and an external layer, referred to herein as a core-shell fiber, wherein the core and/or external layer is segmented/compartmentalized along at least a portion of a length of the fiber. In embodiments, a fiber is comprised of a core, at least one inner shell layer, and at least one external shell layer, referred to herein as annular fibers, wherein any one or more of the core, internal shell layer(s) and/or external shell layer(s) is/are segmented/compartmentalized along at least a portion of a length of the fiber. In embodiments, one or more segments/compartments of a core and/or shell layer(s) may be comprised of a biological material (e.g., cells).

Compartment sizing may be a function of one or more variables, including but not limited to tissue fiber size (e.g., length and/or diameter), type of fiber (e.g., core-shell fiber, annular fiber), type of materials used in the process of tissue fiber generation, whether the generated fiber structure is coated one or more timeset, and the like. In some embodiments, a fiber may be comprised of at least two segments/compartments which include biological material, where other segments flanking the at least two segments/compartments are free of the biological material. For example, in the case of a core-shell fiber, at least two segments comprising biological material may be included within the core. In another example, in the case of an annular fiber, at least two segments comprising biological material may be included within the first shell layer. In embodiments, segment(s) comprising biological material may be of greater length(s) than segment(s) lacking the biological material. In embodiments, segment(s) comprising biological material may be substantially similar in terms of length as compared to segment(s) lacking the biological material. In embodiments, segment(s) comprising biological material may be of lesser length(s) than segment(s) lacking biological material. In embodiments, segment(s) comprising the biological material for particular tissue fibers need not be the same approximate length, but instead different segments may be comprised of different lengths. In embodiments, segment(s) lacking biological material for particular tissue fibers need not be the same approximate length, but different segments may be comprised of different lengths. In some embodiments, spacing between compartments/segments inclusive of biological material (e.g., cells) in a tissue fiber of the present disclosure may be between 1-5 mm, for example 1 mm, 2 mm, 3 mm, 4 mm or 5 mm apart.

Segments/compartments comprised of biological material may comprise, for example, cells of particular densities. In embodiments, the density may be the same between compartments. In embodiments, the density may be different between compartments. In embodiments, the biological material between compartments may be the same, or may be different. In embodiments, density of the biological material may be selected as a function of one or more of particular application (e.g., treatment of particular disease/condition), cell viability determinants, material (e.g., biocompatible material) in which the biological material is included, and the like. As one example, a biological may comprise pancreatic islets. Other biological material (e.g., hepatocytes) may be used in the tissue fibers of the present disclosure at same, or different, densities.

In embodiments, one or more segments/compartments comprising biological material may be flanked by segments that comprise, for example, materials with immunoprotective properties. For illustrative purposes and without limitation, an immunoprotective hydrogel material may comprise, for example, a functionalized alginate including but not limited to methacrylated alginate, alginate furan, alginate thiol, alginate maleimide, and covalent click alginates (e.g., alginate blended with DMAPS-Alg and/or DMAPS-Hzd). For example, in the case of a core-shell fiber in which the core includes two or more segments comprising biological material, the two or more segments may be flanked by other segments that comprise immunoprotective materials as herein disclosed. In other embodiments, the two or more segments comprising biological material may be flanked by other segments that do not comprise, for example, immunoprotective materials, without departing from the scope of this disclosure. Similar logic applies to annular fibers of the present disclosure. For example, an annular fiber may be comprised of two or more segments/compartments which are comprised of biological material, where each of the two or more segments/compartments may be flanked by segments that incorporate, for example, immunoprotective materials of the present disclosure. In other embodiments, the two or more segments comprising biological material may be flanked by segments that do not comprise for example, immunoprotective materials, without departing from the scope of this disclosure.

### Methods of Use

Aspects of the methods comprise providing one or more input materials to be dispensed by way of a dispensing orifice. In some embodiments, one or more cell types are compatible with, and optionally dispensed within, an input material. In some embodiments, a sheath fluid serves as a lubricating agent for lubricating movement of an input material (e.g., within a microfluidic print head). In some embodiments, a sheath fluid comprises a cross-linking agent for solidifying at least a portion of the hydrogel before or while it is dispensed from the dispensing orifice. In some embodiments, a cross-linking agent may be included in an input material, for example an input material corresponding to a core of a fiber of the present disclosure.

Aspects of the methods comprise communicating the design to the 3D printer. In some embodiments, communication can be achieved, for example, by a programmable control processor. In some embodiments, the methods comprise controlling relative positioning of the dispensing orifice and the receiving surface in three dimensional space, and simultaneously dispensing from the dispensing orifice the input material, and, in some embodiments, a sheath fluid, alone or in combination. In some embodiments, the materials dispensed are dispensed coaxially, such that the sheath fluid envelopes the input material. Such coaxial arrangement allows a cross-linking agent in the sheath fluid to solidify the input material, thereby resulting in a solidified fiber structure, which is then dispensed by way of the dispensing orifice.

In some embodiments, a method comprises depositing a first layer of the dispensed fiber structure by way of the means for suspending a bioprinted tissue fiber (e.g., frame 202 at FIG. 2), the first layer comprising an arrangement of the fiber structure specified by the design, and iteratively repeating the depositing step, depositing subsequent fiber structures onto the first and subsequent layers, thereby depositing layer upon layer of dispensed fiber structures in a geometric arrangement specified by the design to produce a 3D structure.

In some embodiments, a plurality of input materials, for example multiple hydrogels, at least some of which comprise one or more cell types, are deposited in a controlled sequence, thereby allowing a controlled arrangement of input materials and cell types to be deposited in a geometric arrangement specified by the design.

In some embodiments, a method comprises removing excess fluid from a receiving surface and/or from the surface of the dispensed fiber structure. For example, the step of removing the excess fluid can be done continuously throughout the printing process, thereby removing excess fluid that may otherwise interfere with layering the dispensed fiber structures in the geometric arrangement provided by the design. Alternatively, the step of removing excess fluid can be done intermittently throughout the printing process in sequence with or simultaneously with one or more depositing steps. In some embodiments, removal of excess fluid is achieved by drawing the fluid off of a receiving surface and/or off of a surface of a dispensed fiber structure. In some embodiments, removal of excess fluid is achieved by drawing excess fluid through a receiving surface or other surface, the surface comprising pores sized to allow passage of the fluid. In some embodiments, removal of excess fluid is achieved by providing a fluid that evaporates after being dispensed from the dispensing orifice.

Aspects of the invention include methods of making a 3D structure comprising one or more input materials. The 3D structures find use in mimicking the normal function of a tissue in a subject which may have become diseased or damaged.

As described above, any suitable divalent cation can be used in conjunction with the subject methods to solidify a chemically cross-linkable input material, including, but not limited to, Cd²⁺, Ba²⁺, Cu²⁺, Ca²⁺, Ni²⁺, Co²⁺, or Mn²⁺. In a preferred embodiment, Ca²⁺ is used as the divalent cation. In one preferred embodiment, a chemically cross-linkable input material is contacted with a solution comprising Ca²⁺ to form a solidified fiber structure. In some embodiments, the concentration of Ca²⁺ in the sheath solution ranges from about 80 mM to about 140 mM, such as about 90, 100, 110, 120 or 130 mM.

In certain embodiments, an input material is solidified in less than about 5 seconds, such as less than about 4 seconds, less than about 3 seconds, less than about 2 seconds, or less than about 1 second.

Aspects of the invention include methods of depositing one or more input materials in a patterned manner, using software tools, to form layers of solidified structures that are formed into a multi-layered 3D tissue structure. In some embodiments, a multi-layered 3D tissue structure comprises a plurality of mammalian cells. Advantageously, by modulating the components (e.g., the mammalian cell type, cell density, matrix components, active agents) of the subject input materials, a multi-layered 3D tissue structure can be created using the subject methods, wherein the multi-layered 3D tissue structure has a precisely controlled composition at any particular location in three dimensional space. As such, the subject methods facilitate production of complex three dimensional tissue structures.

All patents and patent publications referred to herein are hereby incorporated by reference in their entirety.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Bioprinted Fiber Structure

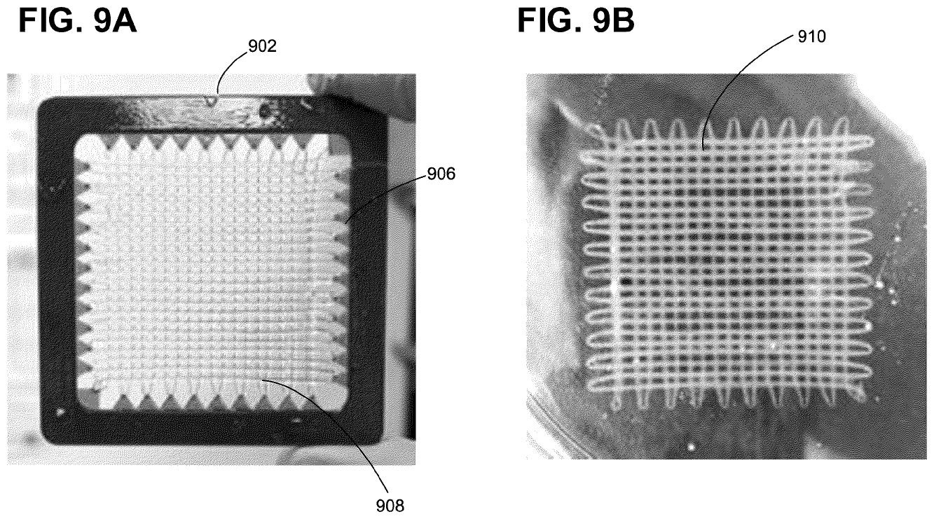

This Example demonstrates a capability to produce a bioprinted fiber structure of the present disclosure, where said structure is suspended during one or more of printing, patterning and/or post-printing processing thereof. FIG. 9A depicts frame 902 (e.g., substantially similar to frame 202 at FIG. 2A), having posts 906 (e.g., substantially similar to posts 206 at FIG. 2A). Additionally shown is fiber structure 908, where said fiber structure was generated by printing the fiber around consecutive opposing posts both vertically and horizontally to generate a lattice-type structure as shown. FIG. 9B is an image of fiber structure 910 following coating an entirety of the bioprinted fiber structure 908 while said structure was fully suspended, followed by its removal from the frame.

### Example 2. Submerged Printing

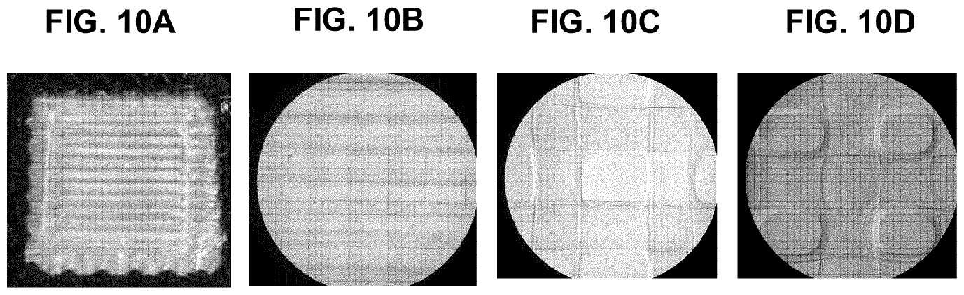

This Example demonstrates that a bioprinted fiber structure can be printed using a means for suspending a bioprinted fiber structure as herein disclosed, where said means is submerged in a crosslinker bath during printing. FIG. 10A illustrates a bioprinted fiber structure that was printed into a cross-linker bath using a means for suspending as herein disclosed, followed by coating. FIG. 10B shows a magnified view of a portion of the structure depicted at FIG. 10A. FIG. 10C illustrates a close-up view of a fiber structure printed into a cross-linker bath, and FIG. 10D illustrates a close-up view of a fiber structure that was not printed into a cross-linker bath, and which was dip-coated following printing. This Example illustrates that devices for which fiber structures are printed in a cross-linker bath by way of the means for suspending (e.g., frames as shown at FIGS. 2A-2D) as herein disclosed have improved fidelity in terms of lesser shape deformation as compared to devices for which the fiber structure is not printed into a cross-linker bath.

### Example 3. Printing Optimization and Viability Study of Mini Device

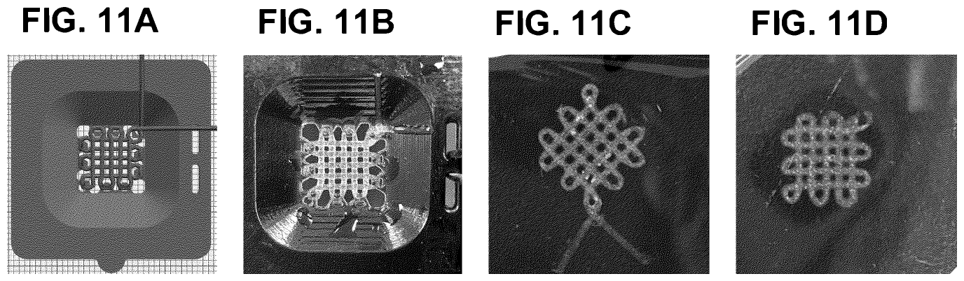

This Example included a test condition of 10×10 mm 4 layer fiber structure against a control condition of 18×18 mm 2 layer fiber structure. FIG. 11A is a schematic of the 10×10 mm fiber structure. FIG. 11B is an image of a coated 10×10 mm fiber structure coupled to a frame. FIGS. 11C-11D depict the coated 10×10 mm fiber structure uncoupled from the frame.

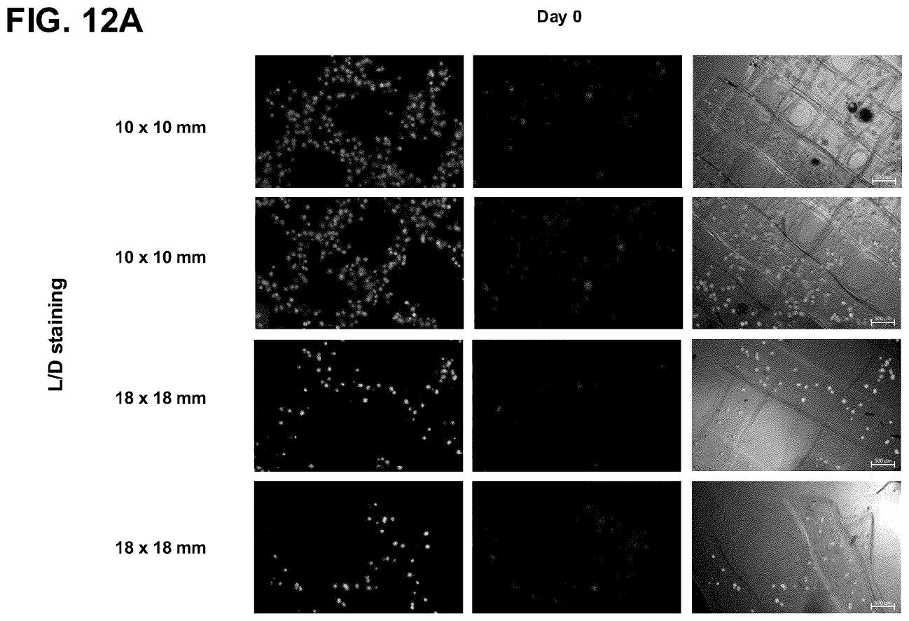

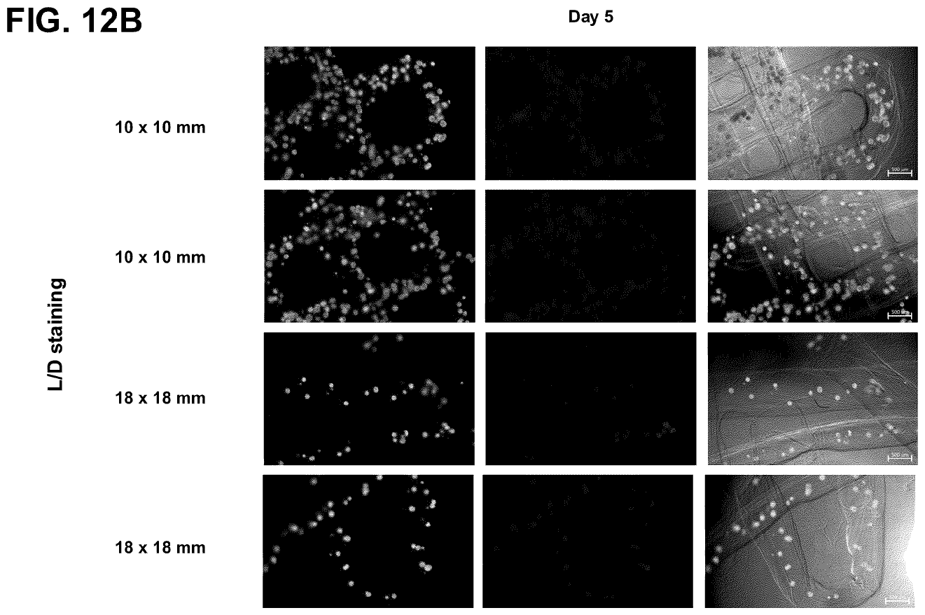

The fiber structures were coated with 0.5% SLG100 (alginate). Cell dose for the structures was 3K IEQ HepG2 aggregates. Live/Dead staining was assessed 0 (FIGS. 12A) and 5 days (FIG. 12B) following printing.

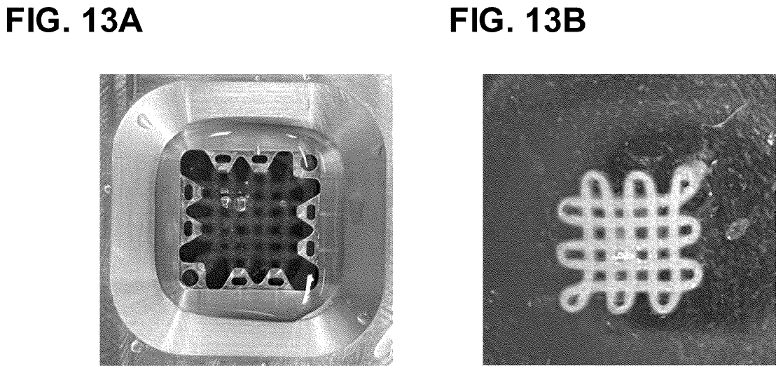

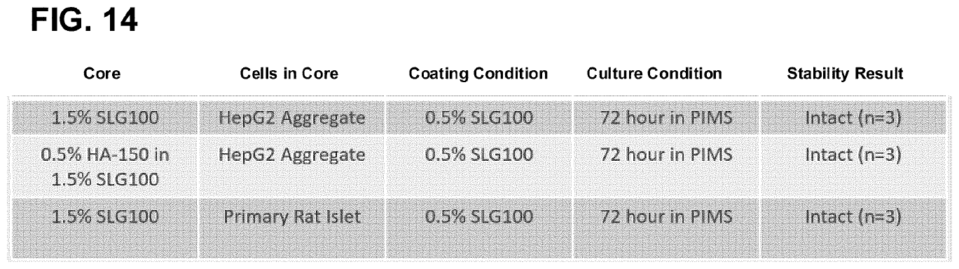

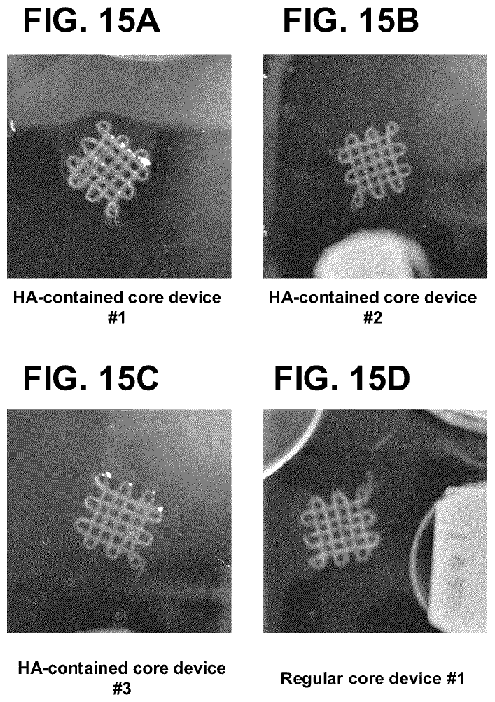

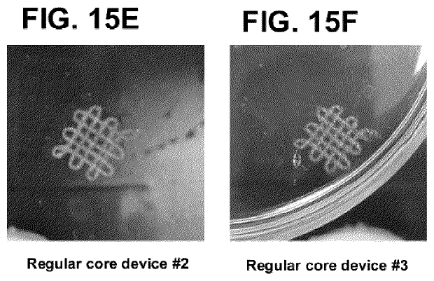

This Example also included testing of the mini device (10×10 mm, 4 layers device coated fiber structure) with a coating of 0.5% SLG100, and a core of either HA-contained 1.5% SLG100 or just 1.5% SLG100. For each case, cell dose was 3K IEQ HepG2 aggregates or primary rat islets (PRI). FIGS. 13A-13B show images of the 10×10 mm fiber structure after coating, on frame (FIG. 13A) and uncoupled from frame (FIG. 13B). The stability data is summarized at FIG. 14. FIG. 15A is an image of structure 1 (HA-contained core), FIG. 15B is an image of structure 2 (HA-contained core), FIG. 15C is an image of structure 3 (HA-contained core), FIG. 15D is an image of structure 1 (regular core), FIG. 15E is an image of structure 2 (regular core), and FIG. 15F is an image of structure 3 (regular core).

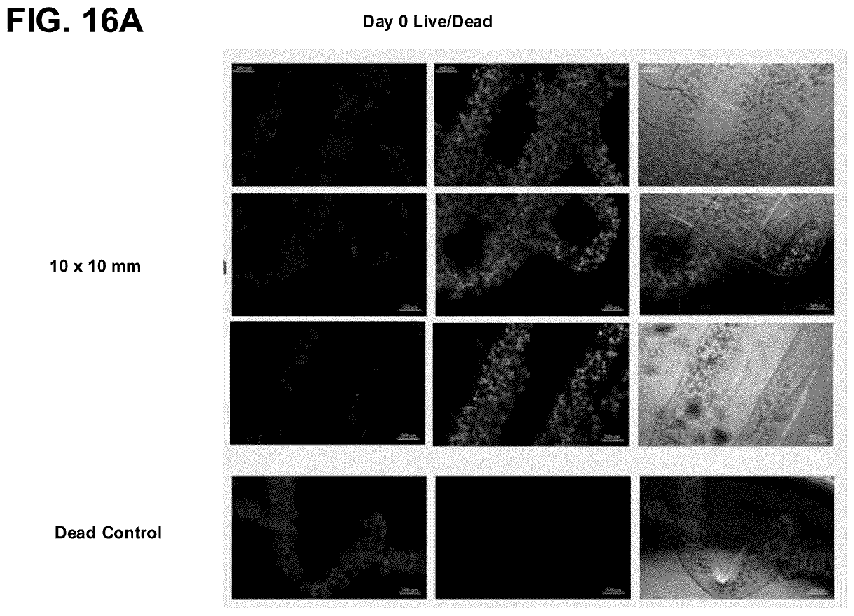

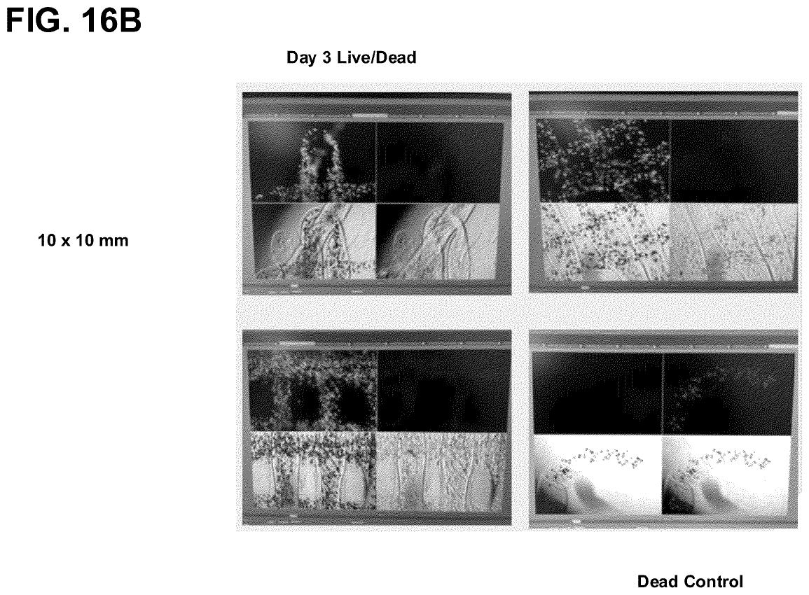

This Example also included testing of coated 10×10 mm fiber structures loaded with PRI (coating 0.5% SLG100, cell dose: 3K IEQ PRI) for viability and functionality. Live/dead staining was assessed at 0 (FIG. 16A) and 3 (FIG. 16B) days post printing.

### Example 4. Frame Vs Mesh Devices Stability Test

This Example demonstrates enhanced stability of a device printed via the use of a frame of the present disclosure, as compared to a device printed in lieu of a frame (i.e., onto mesh without use of a frame).

### Experimental Design

In this Example, the coated fiber structures tested were 18×18 mm, and 2 layers thick. The coated fiber structures included a core (1.5% SLG100), a shell (2% SLG100) and a conformal coating (0.5% SLG100). The core was printed at a flow rate of 115 μL/min, the shell was printed at 80 μL/min, and the sheath flow (i.e., cross-linker solution) was 55 μL/min as dispensed from the print head. Three structures were printed via the use of a frame as herein disclosed, whereas three other structures were printed onto mesh in lieu of a frame. The conformal coating was added to the bioprinted fiber structure while coupled to the frame (devices that relied on the frame for printing), or was added while the bioprinted fiber structure rested on the mesh (devices that were printed in lieu of a frame).

### Stability Test

The stability test was performed as follows. Each coated fiber structure was cultured in a 50 ml conical tube with 15 mL media for 3 days in PIMS media. Each coated fiber structure was subjected to an orbital shaking test at 125 rpm for 30 mins or car transportation for 30 mins. Next each coated fiber structure was poured out into a petri dish, and washed with 10 mL saline three times (saline was aspirated between each rinse). A spatula was used to lift and transfer each coated fiber structure between two petri dishes filled with saline to mimic device transferring on a surgical site. This was repeated 5 times. Finally, each coated fiber structure was transferred onto a moist plastic wrap, and a wand was used to move each device from one edge of the wrap to another side three times (to mimic repositioning of the device on omental membrane).

### Results

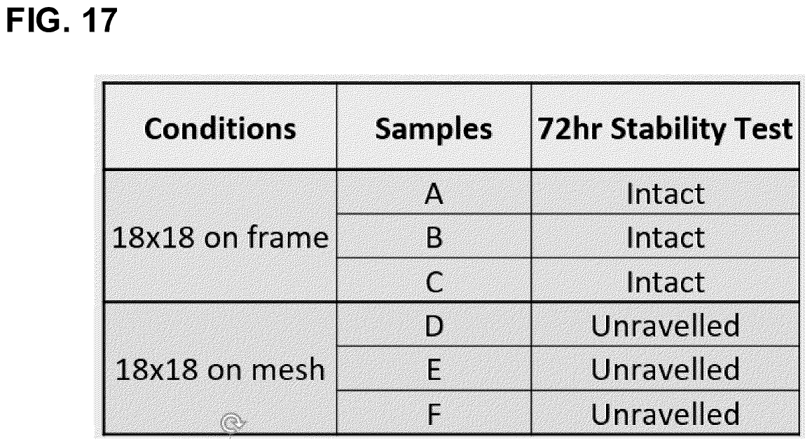

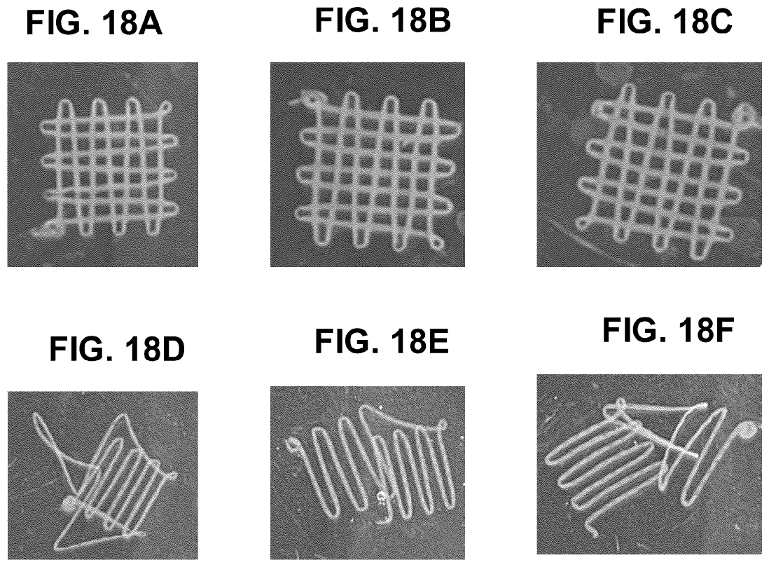

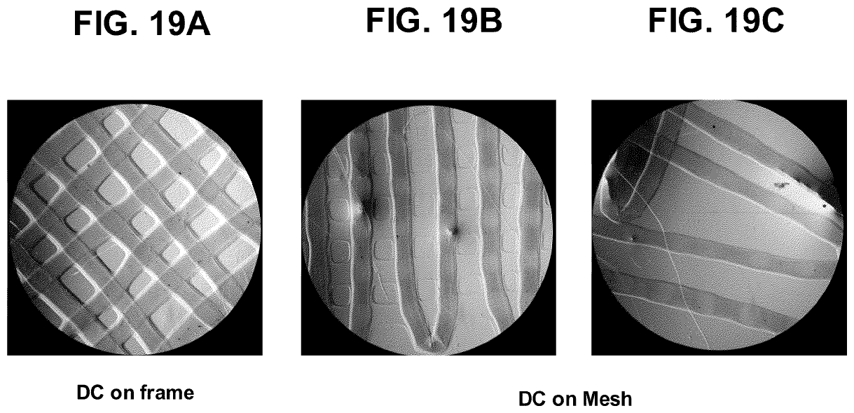

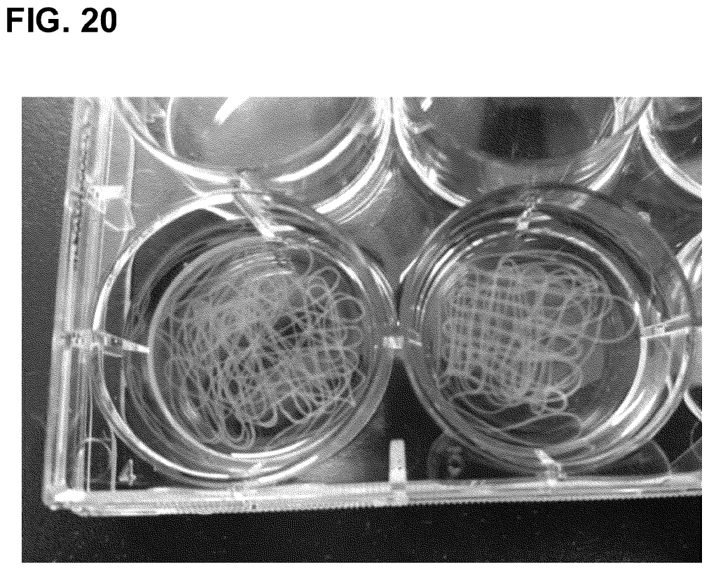

All three coated fiber structures printed and coated via the reliance on a frame as herein disclosed passed the stability test (FIG. 17, and FIGS. 18A-18C as compared to 18D-18F). All three fiber lattice structures printed onto mesh in lieu of a frame failed the stability test, and microscopic images (FIGS. 19A-19C) reveal fibers in the first layer escaped from the coating layer (compare FIGS. PB-PC with that of FIG. 19A). FIG. 20 depicts images of other fiber structures printed onto mesh (but not coated), illustrating degraded stability. The structures of FIG. 20 were created by inside-out cross-linking.

### Example 5. Bioprinted Cell Therapy Platform Normalizes Blood Glucose Control in Diabetic Rat

We have developed microfluidic bioprinting technology that combines biocompatible materials with clinically relevant cells to manufacture implantable tissues for therapeutic applications. Islet cell therapy has been clinically validated for type 1 diabetes (T1D) but relies on lifelong immune-suppression and is limited by the supply of cadaveric donor islets. We are developing a bioprinted pancreatic tissue therapeutic that can deliver allogeneic islets or stem cell-derived pancreatic beta cells into TID patients without the need for immune-suppression, by encapsulation in materials that support physiologic function and shield these cells from direct host immune cell attack.

This Example demonstrates bioprocessing methods to package primary islets into bioprinted tissue implants for in vitro testing and in vivo function studies. We evaluated the ability of bioprinted human islet tissues (xenogeneic) to restore blood glucose control in diabetic, immune-deficient mice and adapted this process for a bioprinted primary rat islet tissue (allogeneic) delivered to the omentum of diabetic rats. Finally, we developed a process to scale this bioprinted pancreatic tissue manufacturing process for delivery of implants in large animals and humans.

### Materials and Methods

The main procedure that was done on the animals is omental device implantation as outlined below. Surgeries were performed post-treatment with STZ.

### A. Prepping the Animal Into Surgery

General concepts of “Rodent Anesthesia” (SOP ACC-01-2017), “Analgesia for Adult Mice and Rats Meloxicam SOP” (TECH 19), and “Local Anesthesia/Analgesia in Adult Mice and Rats Bupivacaine SOP” (TECH 16), were followed. The procedure is explained below:
A1. Place animal in induction chamber on heating pad (temperature should be approximately 38° C.) and induce anesthesia with isoflurane. Flush chamber and move animal to maintenance circuit on nose cone and heat support and maintain isoflurane anesthesia.
A2. Administer a small drop of lubricating eye gel into each eye. Lay the animal flat on its stomach and administer supportive care fluids in the form of 0.9% saline or Lactated Ringers Solution (LRS) at 20 mL/kg subcutaneously. use a 25G with various syringe sizes depending on the dose.
A3. Administer 1 mg/kg Metacam subcutaneously.
A4. Administer buprenorphine 0.05 mg/kg subcutaneously.
A5. While flipping animal over place paper towels under it to catch all the shaved hair. Shave abdominal skin of the animal with a hair clipper.
A6. Take a dry piece of gauze and remove all the hair off and around animal while pulling the paper towel out at the same time.
A7. Once the hair is cleaned up, take a piece of gauze or cotton swab soaked in soap to clean the shaved area. The gauze needs to be moist but not dripping and it will be wrapped around finger. Clean the shaved area in a circular motion and outward, the bubbles should be visible. Leave the soap on the animal for about 30 seconds before wiping down with alcohol.
A8. Wipe the shaved area with gauze or cotton swab in 70% alcohol in an outward circular motion. Go in with a new gauze or cotton swab dipped in soap again but this time scrub/rub the oil on the skin off. Let it sit for 30 second before proceeding to the next step.
A9. Clean the area off with a new gauze or cotton swab soaked in alcohol.
A10. To inject local anesthetic as a line block at the planned incision site. Lift the skin and insert needle subcutaneously under the skin. Pull the needle out as you inject with a “bleb” forming.
A110. Perform one additional skin prep (gauze dipped in soap and again with alcohol). Toe pinch the animal to ensure it is properly anesthetized. Change gloves and wash hands with soap.
A12. Throughout procedure, monitor colour of extremities (should be pink), breathing rate and depth and toe pinch every 5 minutes.

### B. Prepping Surgical Instruments

B1. Don clean exam gloves. This surgery is performed using sterile tip technique, the gloved hands cannot come into contact with any surface that must remain sterile.
B2. Open sterile pack on the counter and aseptically remove the sterile field/wrap that is folded in half, open it up so the inside (sterile field) is placed facing up when placing it close to the surgery table.
B3. Take a pair of sterile forceps to transfer all the instruments and supplies in the pack onto the sterile field wrap. Only the sterile part of the instruments can go on to the sterile field.
B4. Take “Glad Press and Seal Wrap”, throw the first 6″ out, and pull out a piece to place on top of the animal. Avoid using the edges as they are contaminated. Make sure not to touch the top surface with fingers.
B5. Use a pair of forceps to pick up cotton pad to help stick down the Press and Seal onto the animal. To pick up Press and Seal, use a sterile 25G needle to puncture close to the surgery incision area and grab it with forceps to cut a hole over the planned incision site. Do not allow anything non-sterile to come into contact with the top surface of the drape.
B6. Toe pinch the animal with forceps to ensure it is at a surgical level of anesthesia.

### C. Surgical Procedures

General concepts of “Rodent Survival Surgery” (SOP ACC-02-2017) were be followed. All surgical procedures on immune-deficient rats were performed inside a biosafety cabinet of a laminar flow clean air workstation. The procedure is explained below:
C1. Use a pair of toothed forceps to pick up skin. Make a 20 mm incision along the midline of the skin a ˜2 cm below the xiphoid process using a scalpel blade.
C2. Once the skin incision is complete, grasp and lift muscle with the forceps and first make a stab opening using scalpel blade and then make a 20 mm incision with the scissors.
C3. Use a self-retaining tissue retractor to keep the incision to the peritoneal cavity open.
C4. Aseptically place a sterile gauze on the abdominal skin caudal to the opening to prevent direct contact of omental membrane with skin.
C5. Locate the greater omentum and gently stretch the omental membrane out of the opening using a pair of tissue forceps.
C6. Aseptically place the bioprinted implant in the middle of the exposed omentum.
Note: bioprinted implants are composed of a non-reactive, non-rigid polymer with no known bio-incompatibilities. Each device is 20×20×2.5 mm and may contain cells, but is specifically designed to prevent cell release. One device is implanted in each animal. The implants are printed using sterile medical grade ingredients. (The cells are incorporated during the printing process.) Following printing, the devices are be maintained in standard cell culture conditions (culture medium, 37° C./5% CO2) for no more than four days before implantation. Just prior to implantation, the device is washed with a sterile isotonic solution such as saline or Ringer's buffer to remove all traces of the medium.
C7. Fold free ends the omental membrane over the implant and sutured two sides of the omentum using a fine non-absorbable monofilament (e.g., 6-0 Nylon or Prolene), to create a closed pouch.
C8. Gently slide back the omental pouch into the abdominal cavity, and the incision closed with continuous or subcuticular sutures for the muscle and skin layers respectively.
C9. Wet the 5-0 absorbable suture with saline. Use 1 suture pack per rat. Hold the needle perpendicularly with a pair of needle drivers. Make sure the suture does not touch the non-sterile area of the needle holder or forceps.
C10. Insert the needle into one side of the muscle, take it out from the other side of the muscle. When taking the needle out, go with the direction of the needle. Tie a square knot and repeat this knot a total of 3 times. Leave about 2-3 mm of suture and cut the ends. Repeat until muscle areas are closed.
C11. Perform a subcuticular closure of the skin. On one side of the skin, insert the needle just under the skin layer and take it out on a farther side (deeper but still under skin layer) of the skin. Insert the needle on the opposite side of the skin, go from deep to superficial this time. Ensure the needle does not come out of on top of the skin. Tie square knot three times.
C12. Once the skin layer is closed, take a 25G needle and dip into Gluture. Take the needle covered with Gluture to place on top of the sutured skin. Use a pair of forceps to pinch the skin around the needle and slowly take the needle out. The sutured area is now properly closed.

### Recovery of the Rat:

C13. Turn off isoflurane and leave the rat on oxygen via nose cone.
C14. Gently remove the drape and clean any blood off the rat.
C15. Once rat has recovered its righting reflex, place rat into the warmed recovery cage (paper towel covering the bare bottom of the cage).
C16. Monitor the rat in the recovery cage until fully recovered from anesthesia and able to maintain its body temperature without supplemental heat (i.e.: eating, drinking, walking normally, able to climb on a hut, able to groom).
C17. Once fully recovered, return the rat to normal housing cage with cage mates.

### D. Analgesia Plan

D1. Pre-operative Analgesics: Rats will receive a local anesthetic (bupivacaine 0.5 mg, 200 ul of 2.5 mg/ml solution) at the site of incision before tissue is cut, and NSAID (Meloxicam 1 mg/kg, SC), and buprenorphine (0.05 mg/kg, SC) injections. Rats with liver disease induction will receive a combination of oral Ibuprofen and low dose Buprenorphine to account for reduced liver metabolism in these liver disease animals. Oral Ibuprofen (30 mg/kg; resuspension of Ibuprofen liquigel capsule in water by vortexing, protecting from light, replaced every 3 days) will be administered at the time of surgery according to TECH 09b Oral dosing (Gavage) in Rats.
D2. Post-operative Analgesics: Day 1 and Day 2: Meloxicam (1 mg/kg, SC, SID) and buprenorphine (0.02 mg/kg, SC, BID)
TECH 16 (Local Anesthesia/Analgesia in Adult Mice and Rats Bupivacaine SOP) and TECH 19 (Analgesia for Adult Mice and Rats Meloxicam SOP) were followed for these procedures. Rats with liver disease induction will receive a combination of oral Ibuprofen and low dose Buprenorphine to account for reduced liver metabolism in these liver disease animals. Oral Ibuprofen (30 mg/kg; resuspension of Ibuprofen liquigel capsule in water by vortexing, protecting from light, replaced every 3 days) will be administered 6 hours post-surgery according to TECH 09b Oral dosing (Gavage) in Rats. Overnight, the animals continued to receive Ibuprofen in their drinking water (1 mg/mL). At 24 and 48 hours post-surgery, the animals received a subcutaneous injection of Buprenorphine (0.05 mg/kg).

### E. Prophylactic Antibiotic Administration

The bioprinted implants were prepared using sterile materials and reagents and in a sterile condition. All surgical procedures were also performed in an aseptic manner. However, to mitigate any potential risk of infection, prophylactic antibiotics (Enrofloxacin 100 μg/mL in drinking water) was added to drinking water of rats 3 days before surgery until 3 days after surgery. Enrofloxacin is mainly excreted by the kidneys and is also suitable for rats with liver disease.

### Other Procedures

### F. Blood Sampling

Blood samples were collected from rats on a weekly basis until the endpoint of the study. Blood was collected form lateral saphenous vein using TECH 02 SOP “Blood collection from the lateral saphenous vein in mice and rats”. Maximum weekly blood sample volume was 150 ul which is within the allowed limit for sequential blood sampling defined in “UBC ANIMAL CARE COMMITTEE POLICY 006, Policy on Acceptable Methods of Rodent Blood Withdrawal”

### G. Streptozotocin Administration for Induction of Diabetes

Streptozotocin (STZ) destroys the insulin-secreting cells of the pancreas, thereby inducing diabetes. As use of STZ warrants special handling precautions, all users and animal care technicians were made aware of the hazards through the presence of an SDS in the experimental area. Cages containing animals treated with STZ were marked as such for 1 week following STZ injection.
STZ, which is supplied as a powder, is reconstituted in acetate or citrate buffer (pH 4.5) to a concentration of 30 mg/mL just prior to injection.
G1. To induce a model of type 1 diabetes (insulin insufficiency), STZ was injected intraperitoneally rats at a dose of 60 mg/kg in acetate or citrate buffer (a maximum volume of ˜0.5 mL for a standard 250 g rat). TECH 10b (Intraperitoneal Injection in the Adult Rat) was followed for IP injection procedure.
G2. Following injection, the animals'blood glucose levels was monitored daily. Those animals in which sustained hyperglycemia develops (>20 mmol/l blood glucose for two consecutive readings) were used for experiments.
   There is a risk of severe hypoglycemia in the first 24-48 hours. Initial cytotoxic destruction of the beta islet cells causes an excessive release of insulin into the bloodstream. To prevent any fatal hypoglycemia, sucrose water was provided during the induction period to reduce morbidity and mortality. To do so, 10% sucrose was added to drinking water for 48 hours post-STZ injection.

### H. Blood Glucose Measurements

Blood glucose were measured by placing a drop of blood (≤10 μL) onto a glucometer test strip (Lifescan Canada or equivalent).
H1. A small drop of blood was taken from tail vein using TECH 13 (UBC ACC Tail Poke SOP Rat) and applied on the test strip.
H2. Apply gentle pressure with a 2×2 gauze to tip of tail for approximately 10 seconds to stop blood flow.

### I. Oral Glucose Tolerance Test

The main function of pancreatic islets is to secret insulin in response to increase blood sugar levels. To monitor in vivo function of implanted pancreatic islet, we stimulated islets with an oral glucose dose after a short fasting period as described below:
I1. Fast animal for 4 hours (e.g. from 8 AM to 12 PM).
I2. Take a blood sample (fasting, volume: 75 ul)
I3. Dose animal with glucose solution (3 g/kg, 150 ul) through oral gavage using using a 1 mL syringe and a flexible 20 gauge and 38 mm length feeding tube (TECH 09-Oral dosing in Mice and Rats will be followed)
I4. Take another blood sample after 30 min (post-glucose dosing, volume: 75 ul).

### J. Immunofluorescence Protocol for Insulin and CD31

### Materials:

Insulin (C27C9) Rabbit mAb (New England Biolabs), CD31 (PECAM-1) Mouse mAb (New England Biolabs), Anti-rabbit IgG, Alexa Fluor® 647 conjugate (New England Biolabs), Anti-mouse IgG, Alexa Fluor® 488 conjugate (New England Biolabs).

### Procedure:

1. Place slides in a 60° C. oven for 15 min to start deparaffinization process
2. Place slides in a xylene-resistant holder
3. Wash in xylene for 5 min (3×)
4. Wash in 100% EtOH for 5 min (2×)
5. Wash in 95% EtOH for 5 min
6. Wash in 80% EtOH for 5 min
7. Wash in 70% EtOH for 5 min
8. Wash in PBS, on a shaker for 5 min
9. Place slides in a beaker and cover with Antigen Retrieval Buffer (10 mM Citrate Buffer, pH 6.0)
10. Heat on HIGH power for 5 min (2×) in a microwave, making sure the slides are always covered in buffer
11. With heatproof gloves, remove beaker from the microwave and place in sink
12. Cool beaker for 5-10 min under a thin stream of cold running tap water (do not let water hit the slides directly)
13. Wash slides in ddH₂O for 5 min
14. Wash slides in PBS, on a shaker for 5 min
15. Remove excess water on slides and circumscribe samples with hydrophobic pen (Super Pap pen)
16. Block slides 1 hr at room temperature in 5% BSA-PBS with 5% goat serum in humidity chamber
17. Incubate with primary antibodies overnight at 4° C. in humid chamber (diluted in 5% BSA-PBS with 5% goat serum):
   a. Insulin: 1/500 dilution
   b. CD31: 1/500 concentration

### Day 3:

1. Wash slides in PBS for 10 min (3×)
2. Incubate with secondary antibodies at room temperature for 1 hr in dark humid chamber (diluted in 5% BSA-PBS or PBS):
   a. Anti-Mouse: 1/1000 dilution
   b. Anti-Rabbit: 1/1000 dilution
3. From this step forward, slides must be protected from light
4. Wash slides in PBS for 10 min (3×)
5. Mount slides with Fluoroshield with DAPI and seal coverslips with clear nail polish
6. Let dry 24 hours before imaging.

### Islet Processing & In Vitro Bioprinting

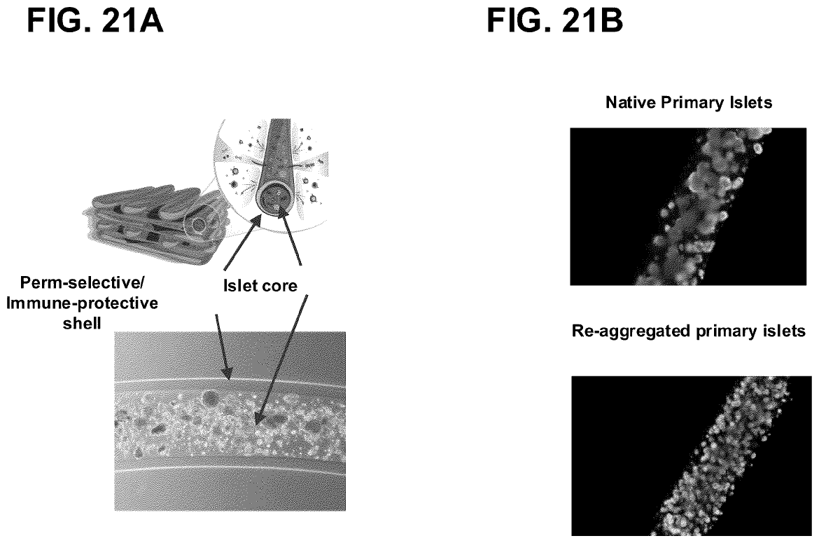

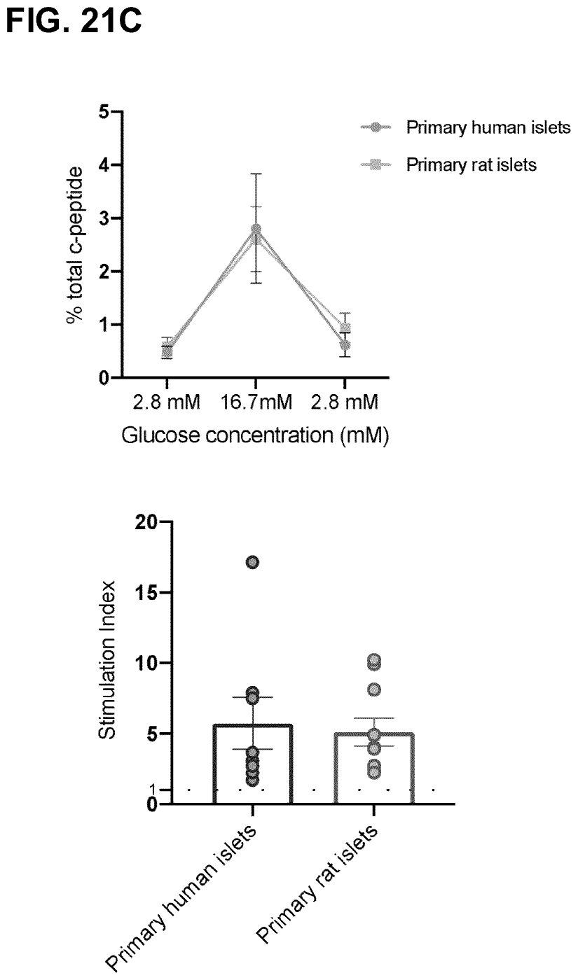

Depicted at FIG. 21A is a schematic representation & bright field image of bioprinted primary human islet tissue. FIG. 21B illustrates live/dead staining of bioprinted primary islets (top: native islets; bottom: re-aggregated islets) after 7 days in culture. FIG. 21C depicts data from a Glucose-Stimulated Insulin Secretion (GSIS) assay performed using primary human (n=8) and rat (n=10) islets; mean+/−SEM.

### Bioprinted Pancreatic Tissue Function in Streptozotocin-Induced Rodent Models of Diabetes

### Immune-Deficient (NSG) Mice: IP Implant

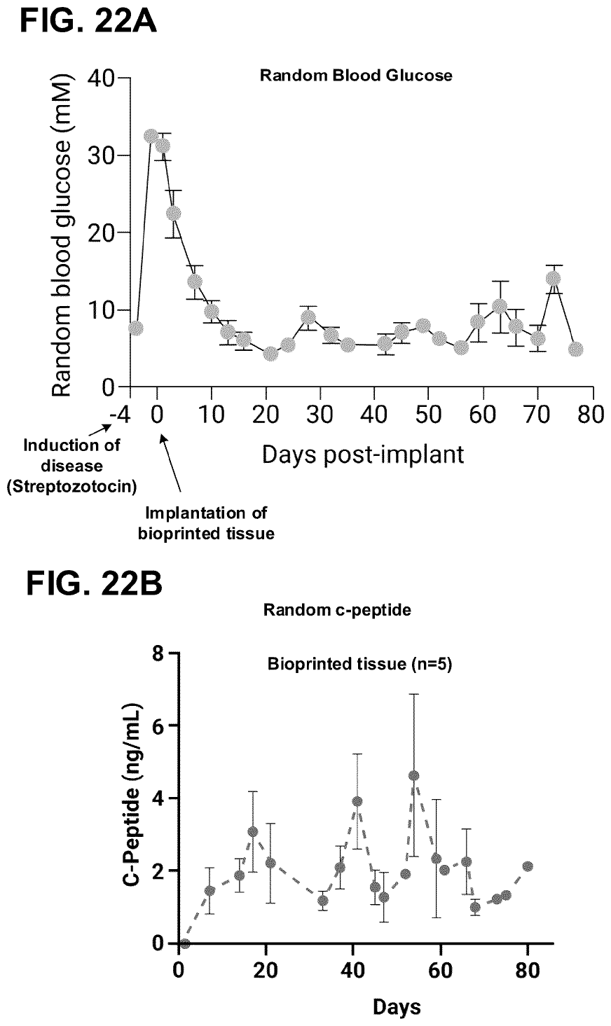

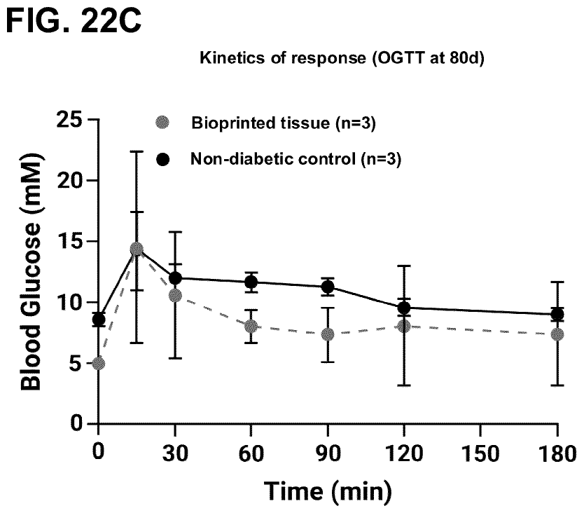

FIG. 22A illustrates random-fed blood glucose measurements following streptozotocin (STZ) treatment and intraperitoneal (IP) implantation of bioprinted human islet tissue in NSG (NOD scid gamma) mice (n=5) over 80 days; Day 0 represents time of implantation. FIG. 22B shows human C-peptide levels measured in mouse plasma over 80 days using ELISA. FIG. 22C illustrates data from an oral glucose tolerance test (OGTT) performed at day 80 to assess kinetics of blood glucose normalization following a fasting period and subsequent glucose challenge in NSG mice with bioprinted islet tissues or healthy, non-STZ treated control mice.

### Immune-Deficient (Nude) Rats: Omental Implant

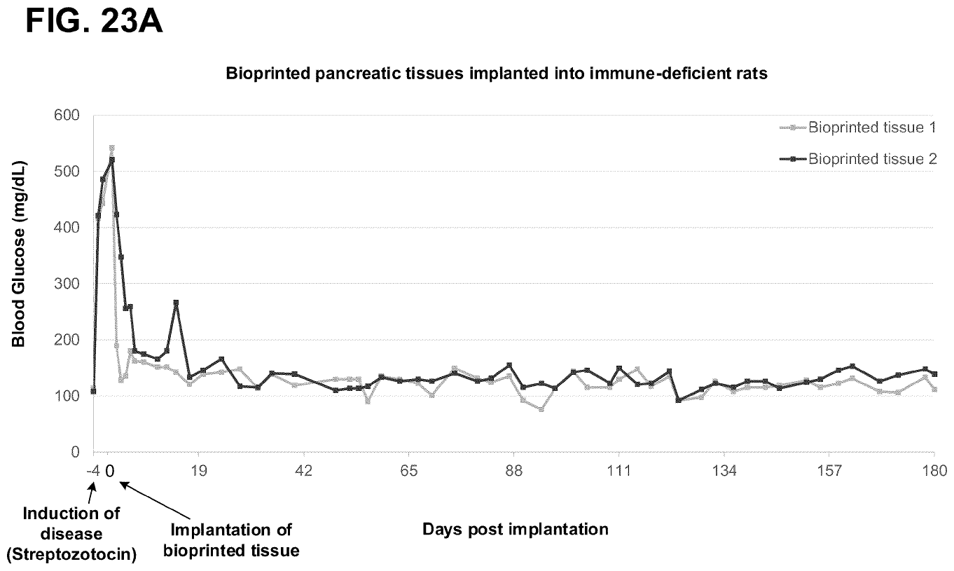

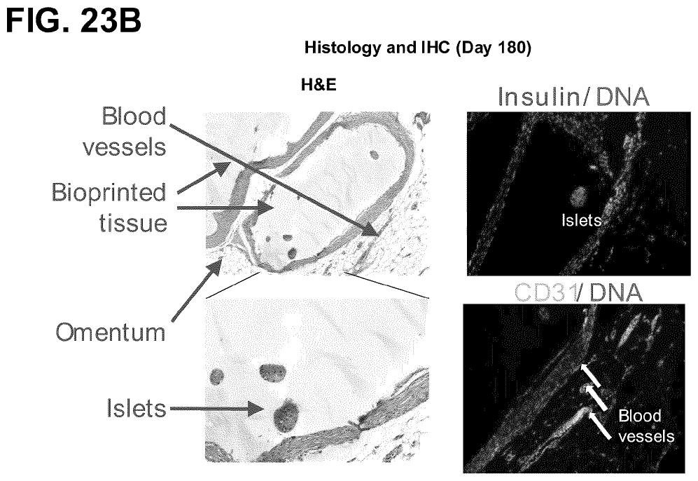

FIG. 23A illustrates blood glucose measurements following omental pouch implantation of bioprinted rat islet tissue in STZ-treated nude rats (n=2) over 180 days. FIG. 23B shows H&E (high and low magnification) and insulin (islets) or CD31 (endothelial cells) immunohistochemistry (IHC) performed on sections from fixed, bioprinted tissue explanted at 180 days.

### Immune-Competent (Sprague-Dawley) Rats: Omental Implant

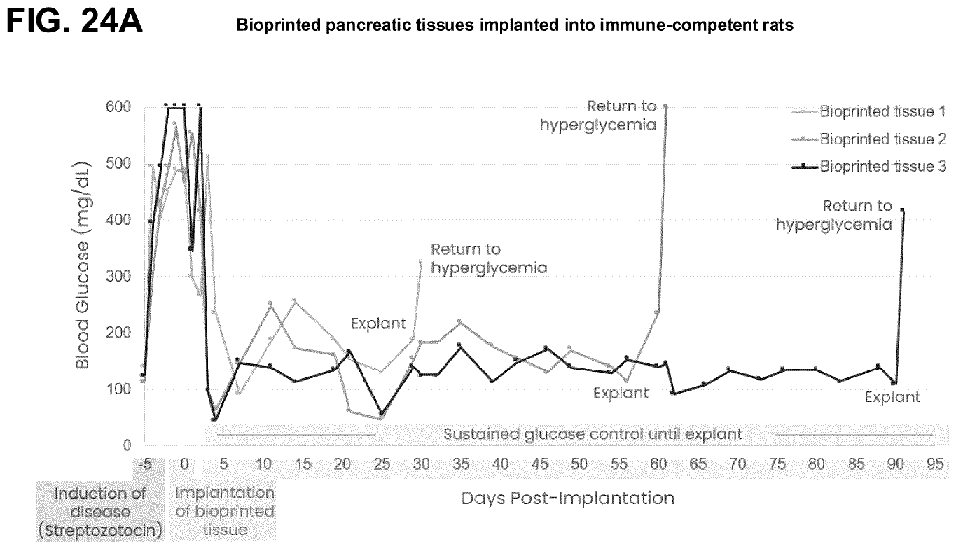

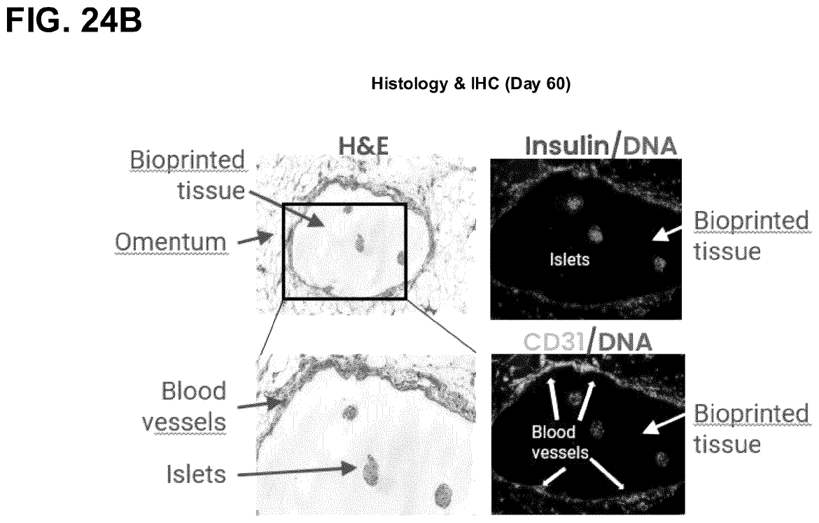

FIG. 24A illustrates blood glucose measurements following omental pouch implantation of bioprinted Lewis rat islet tissue in STZ-treated Sprague-Dawley (SD) rats (n=3) over 90 days; explant retrieval and return to hyperglycemia was performed at 30, 60, and 90 days following surgical implantation. FIG. 24B shows H&E and insulin (islets) or CD31 (endothelial cells) IHC performed on sections from fixed, bioprinted tissue explanted at 60 days.

### Bioprinted Pancreatic Tissue Scale-Up for Large Animals

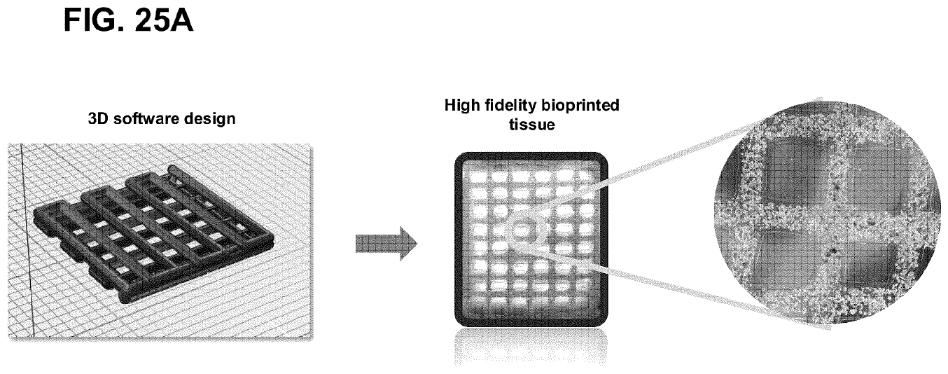

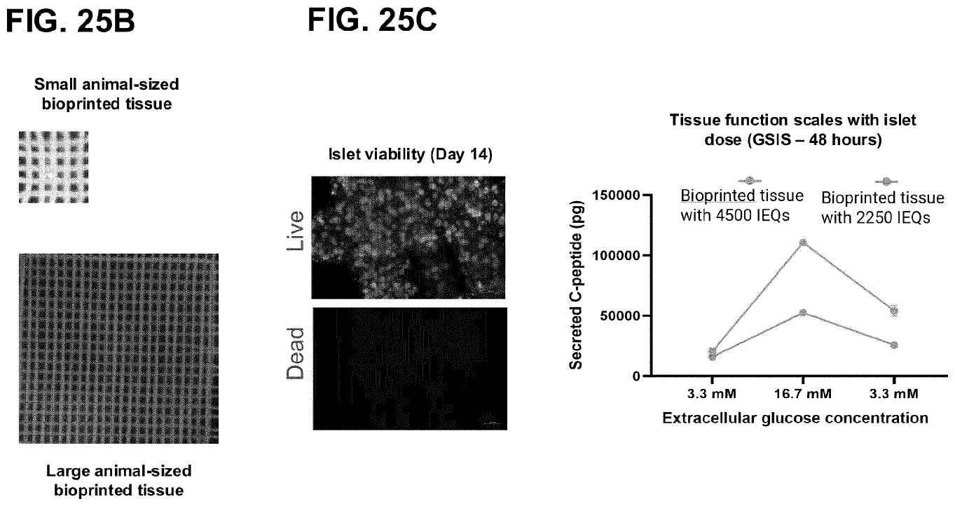

FIG. 25A is a schematic illustrating that the biomanufacturing process involves tissue design in proprietary software and QC of bioprinted tissues, including confirmation of micro- and macro-architectures, cell viability, and islet distribution. FIG. 25B shows bioprinted pancreatic tissue used for studies in rats compared to scaled-up tissue for large animals. FIG. 25C shows viability of bioprinted neonatal porcine islets confirmed up to 14 days post-print. GSIS demonstrates bioprinted tissue function scales with the dose of human islets.

### Conclusion

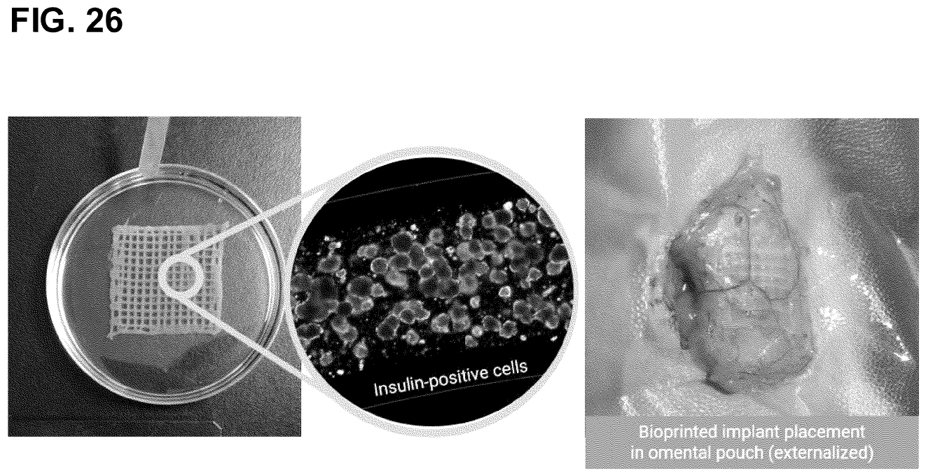

We developed a process (see FIG. 26) to manufacture implantable tissues containing bioprocessed pancreatic islets in materials that protect these allogeneic cells from host immune cell attack. This Example shows that the bioprinted pancreatic tissues: 1) maintain islet viability and function in vitro, 2) restore blood glucose control in mouse and rat models of diabetes, 3) support islet function and immune-protection over 90 days in a rat model of diabetes, and 4) can be scaled up for large animal studies and ultimately, delivery into TID patients.

### Example 6. Double Dip Coating of a Bioprinted Fiber Structure

In this Example, the coated fiber structures tested were 16×16 mm, and 2 layers thick and printed on a device of the present disclosure that included a vessel as described herein. The coated fiber structures included a core (1.5% SLG100 with cells or dye), a shell (2% SLG100), an inner conformal coating (2% SLG100) and an outer conformal coating (2% Zwit-20 Alginate). The structures were printed via the use of a frame as herein disclosed. The inner and outer conformal coatings were added to the bioprinted fiber structure while coupled to the frame (devices that relied on the frame for printing).

After printing of the 2-layer, 16×16 mm fiber structures, the structures were cross-linked with 95%/5% Ca/Ba in 15% polyethylene glycol (PEG) in pH-buffered dH₂O for 3 minutes. The cross-linking bath was removed by vacuuming and the fiber structures were rinsed with TSC saline from the buffer channel on the printhead. All solution was then removed by vacuuming and the fiber structures were raised 3 to 5 mm from the receiving surface.

A first conformal coating solution of 1 mL of 2% SLG100 was then pipetted into the vessel to evenly coat the entire fiber structure, ensuring that all fibers were covered with the solution from both the top and bottom. The fiber structure was incubated in the first conformal coating solution for 10 seconds after which the extra solution was removed by vacuum from the vessel.

A second conformal coating solution of 1 mL of Zwit-20 alginate was then pipetted into the vessel to evenly coat the entire fiber structure, ensuring that all fibers were covered with the solution from both the top and bottom. The fiber structure was incubated in the second conformal coating solution for 35 seconds after which the extra solution was removed by vacuum from the vessel.

The fiber structure was then transferred to a 95%/5% Ca/Ba bath and allowed to cross-link for 3 minutes after which the fiber structure was rinsed with saline.

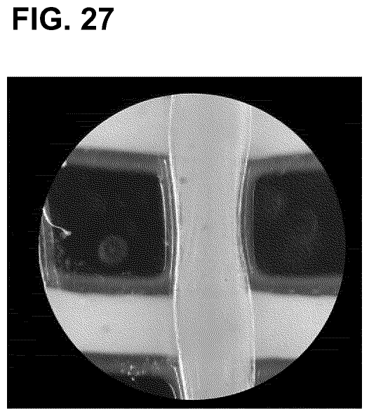

Photographs of the fiber structure with the inner and outer conformal coating were taken and are shown in FIG. 27. The diameter of the entire vertical fiber with both conformal coatings as depicted in FIG. 27 was 1.004 mm, the inner conformal coating was between 40.8 μm (left side, midpoint) and 50 μm (right side, midpoint) and the outer conformal coating was between 88.1 μm (left side, midpoint) and 90 μm (right side, midpoint).

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

1. Fabrication platform for supporting a bioprinted fiber structure during printing, patterning and/or processing, wherein said platform comprises a frame defining a void and comprising a plurality of posts on opposing sides of the frame for securing and suspending at least one cross-linkable fiber within said frame to form said fiber structure, wherein a continuous length of said at least one fiber is printed around at least two, three, four, five, six, seven, eight, nine, ten or more of said posts during the 3D bioprinting process.

2. The fabrication platform of claim 1, wherein said posts are positioned on the interior of said frame; preferably wherein said posts are positioned on frame projections extending into said void.

3. The fabrication platform of claim 2, wherein said posts are evenly or unevenly spaced around said frame.

4. The fabrication platform of claim 1, wherein said frame comprises at least 10, 15, 20, 25, 30, 35, 40, 45, 50, 55, 60, 65, 70, 75, 80, 85, 90, 95, or 100 posts.

5. The fabrication platform of claim 1, wherein at least a portion of said cross-linkable fiber comprises a biological material.

6. The fabrication platform of claim 1, wherein said frame is square, rectangular, triangular, hexagonal, octagonal, circular, or irregular.

7. The fabrication platform of claim 1, wherein said fiber structure comprises a lattice.

8. The fabrication platform of claim 6, wherein said frame is coupled to a mounting bracket configured for adjusting a position of said frame with respect to a receiving surface.

9. The fabrication platform of claim 1, wherein said frame further comprises a fiber cutting groove positioned alongside the void to permit a cutting implement to cut a portion of the fiber structure.

10. The fabrication platform of claim 1, further comprising a mating groove disposed on a bottom surface of the frame and configured to receive the walls of a container or vessel on the receiving surface.

11. Means for suspending a bioprinted fiber structure during printing, patterning and/or processing, wherein said means for suspending comprises a frame coupled to a mounting bracket and/or a receiving surface of a bioprinting system, said frame comprising a plurality of posts encircling the frame for securing a continuous length of at least one cross-linkable fiber forming said fiber structure.

12. A bioprinting system comprising:
the fabrication platform according to any one of claims 1-10, or the means for suspending according to claim 11;
at least one dispensing orifice for dispensing said at least one crosslinkable fiber onto said receiving surface;
a positioning unit for positioning the receiving surface in three dimensional space with respect to the dispensing orifice, the positioning unit operably coupled to either the receiving surface or the at least one dispensing orifice; and
a dispensing means for dispensing the at least one cross-linkable fiber from the at least one dispensing orifice.

13. The bioprinting system of claim 12, wherein said fabrication platform or said means for suspending is suspended over said receiving surface.

14. The bioprinting system of claim 12, wherein said receiving surface comprises a porous material.

15. The bioprinting system of claim 12, wherein said receiving surface comprises a vessel containing a liquid.

16. The bioprinting system of claim 13, further comprising a programmable control processor for controlling the positioning component and for controlling a flow rate of one or more fluids via the dispensing means.

17. The bioprinting system of claim 13, wherein said dispensing means comprises at least one pump; optionally wherein said at least one pump comprises a pump assembly comprising a plurality of pumps positioned in a radial array on a mounting bracket.

18. The bioprinting system of any one of claims 12-17, further comprising at least one print head comprising a plurality of microfluidic printing channels to selectively provide a respective plurality of materials.

19. The bioprinting system of any one of claims 12-18, further comprising a vacuum chuck disposed on the receiving surface and an integrated container formed by walls projecting from the top surface of the vacuum chuck and defining the perimeter of the container, preferably wherein the walls are configured to insert into a mating groove on the bottom of the frame.

20. A method for bioprinting a fiber structure, the method comprising:
providing a system according to claim 12, and dispensing a continuous length of the cross-linkable fiber around a plurality of said posts on said frame of said fabrication platform to generate the fiber structure.

21. The method of claim 20, further comprising:
adding a conformal coating to an entirety of an external surface of the fiber structure while said fiber remains coupled to said frame.

22. The method of claim 20 or claim 21, further comprising:
transporting the fiber structure from one location to another while said fiber structure remains coupled to said frame.

23. The method of claim 20 or claim 21, further comprising:
storing said fiber structure while said fiber structure remains coupled to said frame.

24. A bioprinted fiber structure made by a method according to claim 20, comprising a continuous length of the cross-linkable fiber comprising at least one biological material, wherein said cross-linkable fiber comprises a solid core and at least one external shell layer surrounding said solid core, wherein said bioprinted fiber structure comprises at least two layers of a lattice/grid formed by said continuous cross-linkable fiber.

25. The bioprinted fiber structure according to claim 24, wherein each layer is about 0.050 to about 3 mm thick.

26. The bioprinted fiber structure according to claim 25, having an infill density of between about 10% and about 90%, or between about 20% and about 80%, or between about 30% and about 70%, or between about 40% and about 60%, preferably about 30%, about 40%, about 50%, or about 60%.

27. The bioprinted fiber structure according to claim 24, having a fiber to fiber distance of between about 1000 to 2000 μm, such as about 1400 to 1600 μm, or about ˜1500 μm.

28. The bioprinted fiber structure according to claim 24, wherein said solid core comprises said at least one biological material; optionally wherein said solid core is compartmentalized along the length of said fiber.

29. The bioprinted fiber structure according to claim 24, further comprising at least one internal shell layer surrounding said solid core, said at least one internal shell layer comprising at least one biological material; optionally wherein said solid core and/or said at least one internal shell layer is/are compartmentalized along the length of said fiber.

30. The bioprinted fiber structure according to any of claims 24-29, further comprising at least one conformal coating.

31. The bioprinted fiber structure according to claim 30, comprising a single conformal coating.

32. The bioprinted fiber structure according to claim 31, wherein said solid core comprises about 0.75-1.5% alginate, said at least one external shell layer comprises about 1.5-2.5% alginate, and said coating comprises about 0.2-0.75% alginate.

33. The bioprinted fiber structure according to claim 30, comprising an inner conformal coating and an outer conformal coating.

34. The bioprinted fiber according to any of claims 24-33, wherein said at least one biological material comprises pancreatic islet cells.
